(19) United States
(12) Patent Application Publication
(10) Pub. No.: US 2025/0205198 A1
(43) Pub. Date: Jun. 26, 2025

(54) METHODS FOR DELIVERY OF PSYCHEDELIC MEDICATIONS BY INHALATION AND SYSTEMS FOR PERFORMING THE METHODS

(71) Applicant: Cybin IRL Limited, Dublin (IE)

(72) Inventors: Michael Palfreyman, St. Petersburg, FL (US); Alex Nivorozhkin, West Roxbury, MA (US); Brett J. Greene, Fitchburg, MA (US); Robert Mino, Jupiter, FL (US)

(73) Assignee: Cybin IRL Limited, Dublin (IE)

(21) Appl. No.: 18/850,356

(22) PCT Filed: Mar. 28, 2023
(86) PCT No.: PCT/EP2023/057939

§ 371 Date: Sep. 24, 2024

Related U.S. Application Data
(60) Provisional application No. 63/362,238, filed on Mar. 31, 2022.

(51) Int. Cl. A61K 31/4045; A61K 9/00; A61K 9/12; A61K 47/02

(52) U.S. Cl. CPC A61K 31/4045; A61K 9/0078; A61K 9/12; A61K 47/02

(57) ABSTRACT

Provided are methods for delivering psychedelic drugs such as tryptamine psychedelics to a patient in need thereof via inhalation. The psychedelic drug is delivered in the form of an aerosol, such as a mist or a dry powder. Methods for treating a central nervous system (CNS) disorder or psychological disorder via inhalation routes are provided.

### CROSS-REFERENCE

This application claims priority to U.S. Provisional Application No. 63/362,238, filed on Mar. 31, 2022, which is incorporated by reference herein in its entirety.

### BACKGROUND

### Field

Provided are methods for delivery of psychedelic medications by inhalation and systems and devices for performing those methods.

### Description of the Related Art

Psychedelic compounds, both natural and synthetic, such as tryptamines, phenethylamines, ergolines and other derivatives, possess a range of valuable therapeutic properties that could be useful in treatments.

Psychedelics are named such because of their experiential effects on the user. Most often, the psychedelic experience acts to enhance the mood of the user when consumed. However, one potential psychological disorder resulting from the administration of psychedelics as therapeutics is the risk of a negative experience for the patient, presenting as acute psychedelic crisis, colloquially known as a “bad trip,” in which the patient experiences feelings of remorse or distress.

The therapeutic index of many psychedelics is relatively narrow. Therefore, maximizing therapeutic benefits of potential drug candidate molecules requires fine-tuning of the dose and route of administration, along with dose titration, to reduce the side effects and improve safety.

In most cases the standard route of administration is by oral delivery, which is often times complicated by metabolic transformation leading to both decreased efficacy and increased toxicity. For some compounds, the oral route is completely ineffective. For example, a naturally occurring dimethyltryptamine (DMT) is orally inactive unless when combined with MAO inhibitors as in the folk medicine ayahuasca.

Inhalation methods of drug administration is generally geared toward relatively common medical situations including asthma, pain control or treatment of diabetes. Pulmonary delivery is attractive as a route for systemic administration due to fast absorption by the massive surface area of the alveolar region, the abundant vasculature and thin air-blood barrier, and the avoidance of first pass metabolism. The effectiveness of an aerosol therapy is largely dependent on how much of the medication will reach the intended site of deposition. The deposition pattern of the administered aerosol is determined mainly by the formulation and the delivery device. Accordingly, there is an unmet medical need for methods and devices to deliver psychedelic drugs by inhalation, where the dosage can be controlled, while delivering the drug systemically via the pulmonary system.

### SUMMARY

Accordingly, one object is to provide methods for delivery of a psychedelic medication or drug (including a combination of medications or drugs) via inhalation to a patient in need thereof.

A further object is to provide methods for delivery of a psychedelic medication or drug (including a combination of medications or drugs) via inhalation, in order to rapidly deliver the psychedelic drug to the blood stream, bypassing first-pass metabolism.

Another object is to provide a method for treatment of central nervous system disorders or psychological disorders by administration of a mixture of nitrous oxide (or noble gas such as xenon and/or argon) and oxygen (or air).

A further object is to provide a method for delivery of a psychedelic drug by co-administration of the psychedelic drug and nitrous oxide (or noble gas such as xenon and/or argon) by inhalation where the nitrous oxide (or noble gas) acts a driving gas for nebulization of the psychedelic drug.

A further object is to provide a medical device for the coadministration by inhalation of a psychedelic drug and nitrous oxide (or noble gas).

A further object is to provide a nitrous oxide (or noble gas) and psychedelic drug delivery device that can be remotely activated and regulated to control the dose and duration of treatment, thus providing at home treatments under supervision of the therapist/psychiatrist via telehealth, thus assisting with patient compliance in drug administration and helping to prevent overdosing.

These, and other objects, alone or in combinations, have been satisfied by the discovery of methods for delivering psychedelic drugs to a patient in need thereof comprising administering via inhalation of a psychedelic drug in the form of an aerosol, methods for treating a central nervous system (CNS) disorder or psychological disorder via inhalation of a psychedelic drug in the form of an aerosol, devices for delivery of psychedelic drug and nitrous oxide (or noble gas) mixtures by inhalation, including with remote activation and control, and methods for treating a central nervous system (CNS) disorder or psychological disorder via inhalation of nitrous oxide (or noble gas)/oxygen mixtures e.g., those having an amount of nitrous oxide (or noble gas) of 15 to 25% by volume of total gas.

Thus, the following embodiments, which are not intended to be limiting are disclosed:

Embodiment 1. A method of delivering a psychedelic drug to a patient in need thereof, comprising administering an aerosol to the patient by inhalation, wherein the aerosol comprises the psychedelic drug in a carrier, and the psychedelic drug is a compound of Formula (I) or a pharmaceutically acceptable salt, stereoisomer, solvate, or prodrug thereof

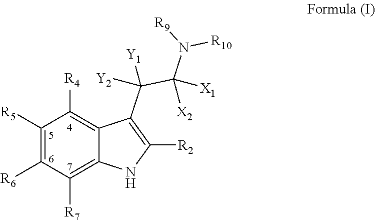

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

Embodiment 2. The method of Embodiment 1, wherein at least one of X₁, X₂, Y₁, Y₂, R₂, R₄, R₅, R₆, R₇, R₉, and R₁₀ comprises deuterium.

Embodiment 3. The method of Embodiment 1 or 2, wherein X₁, X₂, R₉, and R₁₀ comprise deuterium.

Embodiment 4. The method of any one of Embodiments 1 to 3, wherein X₁, X₂, Y₁, Y₂, R₉, and R₁₀ comprise deuterium.

Embodiment 5. The method of any one of Embodiments 1 to 4, wherein X₁, X₂, and R₅ comprise deuterium.

Embodiment 6. The method of any one of Embodiments 1 to 5, wherein X₁, X₂, Y₁, Y₂, R₅, R₉, and R₁₀ comprise deuterium.

Embodiment 7. The method of any one of Embodiments 1 to 6, wherein the compound of Formula (I) is at least one selected from the group consisting of 2-(1H-indol-3-yl)-N,N-bis(methyl-d₃)ethan-1-amine-1,1,2,2-d₄; 2-(1H-indol-3-yl)-N,N-bis(methyl-d₃)ethan-1-amine-1,1-d₂; 2-(5-methoxy-1H-indol-3-yl)-N,N-bis(methyl-d₃)ethan-1-amine-1,1,2,2-d₄; 2-(5-(methoxy-d₃)-1H-indol-3-yl)-N,N-dimethylethan-1-amine-1,1-d₂; and 2-(5-(methoxy-d₃)-1H-indol-3-yl)-N,N-bis(methyl-d₃)ethan-1-amine-1,1,2,2-d₄; or a pharmaceutically acceptable salt, solvate, or prodrug thereof.

Embodiment 8. The method of any one of Embodiments 1 to 7, wherein the psychedelic drug is a fumarate salt, benzoate salt, salicylate salt, or succinate salt of at least one selected from the group consisting of 2-(1H-indol-3-yl)-N,N-bis(methyl-d₃)ethan-1-amine-1,1,2,2-d₄; 2-(1H-indol-3-yl)-N,N-bis(methyl-d₃)ethan-1-amine-1,1-d₂; 2-(5-methoxy-1H-indol-3-yl)-N,N-bis(methyl-d₃)ethan-1-amine-1,1,2,2-d₄; 2-(5-(methoxy-d₃)-1H-indol-3-yl)-N,N-dimethylethan-1-amine-1,1-d₂; and 2-(5-(methoxy-d₃)-1H-indol-3-yl)-N,N-bis(methyl-d₃)ethan-1-amine-1,1,2,2-d₄.

Embodiment 9. The method of any one of Embodiments 1 to 8, wherein the psychedelic drug is a psychedelic drug mixture of at least two compounds of Formula (I), the psychedelic drug mixture comprising (i) 2-(1H-indol-3-yl)-N,N-bis(methyl-d₃)ethan-1-amine-1,1,2,2-d₄, or a pharmaceutically acceptable salt, solvate, or prodrug thereof; (ii) one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof.

Embodiment 10. The method of Embodiment 9, wherein the psychedelic drug mixture comprises (i) from 60% to 99% by weight of 2-(1H-indol-3-yl)-N,N-bis(methyl-d₃)ethan-1-amine-1,1,2,2-d₄, or a pharmaceutically acceptable salt, solvate, or prodrug thereof, based on a total weight of the psychedelic drug mixture; (ii) from 1% to 40% by weight, in sum, of one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof, based on a total weight of the psychedelic drug mixture; and (iii) from 0% by weight to less than 10% by weight, in sum, of one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof, based on a total weight of the psychedelic drug mixture.

Embodiment 11. The method of any one of Embodiments 1 to 10, wherein the carrier is air, oxygen, or a mixture of helium and oxygen.

Embodiment 12. The method of Embodiment 11, wherein the carrier is the mixture of helium and oxygen.

Embodiment 13. The method of Embodiment 12, wherein the mixture of helium and oxygen is heated to about 50° C. to about 60° C.

Embodiment 14. The method of Embodiment 12 or 13, wherein the helium is present in the mixture of helium and oxygen at about 50 to 90% by volume, and the oxygen is present in the mixture of helium and oxygen at about 10 to 50% by volume.

Embodiment 15. The method of any one of Embodiments 1 to 14, further comprising administering a pretreatment inhalation therapy prior to administration of the aerosol comprising the psychedelic drug and the carrier.

Embodiment 16. The method of Embodiment 15, wherein the pretreatment inhalation therapy comprises administering via inhalation a mixture of helium and oxygen heated to about 90° C. to about 120° C. to the patient.

Embodiment 17. The method of any one of Embodiments 1 to 16, wherein the psychedelic drug is delivered to the patient's central nervous system, providing an improvement in drug bioavailability by at least 25% as compared to oral delivery, increased Cₘₐₓ by at least 25% as compared to oral delivery, reduced Tₘₐₓ by at least 50% as compared to oral delivery, or a combination thereof.

Embodiment 18. The method of any one of Embodiments 1 to 17, wherein the aerosol is a mist.

Embodiment 19. The method of any one of Embodiments 1 to 18, wherein the aerosol is prepared by nebulization of the psychedelic drug.

Embodiment 20. The method of Embodiment 19, wherein the nebulization is performed with a device selected from the group consisting of a jet nebulizer, an ultrasonic nebulizer, a breath-actuated nebulizer, and a vibrating mesh nebulizer.

Embodiment 21. The method of Embodiment 19 or 20, wherein the nebulization is performed using nitrous oxide as a driving gas for entrainment of the psychedelic drug in nebulized form.

Embodiment 22. The method of Embodiment 21, wherein the nitrous oxide is present in a concentration of 15 to 25% of a volume of gas used.

Embodiment 23. The method of any one of Embodiments 1 to 22, wherein the aerosol is administered for 20 to 60 minutes.

Embodiment 24. A method of treating a patient with a central nervous system (CNS) disorder or psychological disorder comprising administering to the patient, via inhalation, an aerosol comprising a psychedelic drug in a carrier, wherein the psychedelic drug is a compound of Formula (I) or a pharmaceutically acceptable salt, stereoisomer, solvate, or prodrug thereof

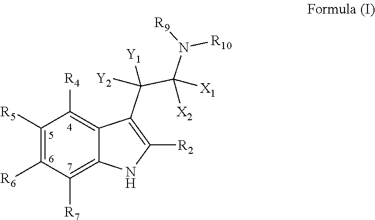

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

Embodiment 25. The method of Embodiment 24, wherein at least one of X₁, X₂, Y₁, Y₂, R₂, R₄, R₅, R₆, R₇, R₉, and R₁₀ comprises deuterium.

Embodiment 26. The method of Embodiment 24 or 25, wherein X₁, X₂, R₉, and R₁₀ comprise deuterium.

Embodiment 27. The method of any one of Embodiments 24 to 26, wherein X₁, X₂, Y₁, Y₂, R₉, and R₁₀ comprise deuterium.

Embodiment 28. The method of any one of Embodiments 24 to 27, wherein X₁, X₂, and R₅ comprise deuterium.

Embodiment 29. The method of any one of Embodiments 24 to 28, wherein X₁, X₂, Y₁, Y₂, R₅, R₉, and R₁₀ comprise deuterium.

Embodiment 30. The method of any one of Embodiments 24 to 29, wherein the compound of Formula (I) is at least one selected from the group consisting of 2-(1H-indol-3-yl)-N,N-bis(methyl-d₃)ethan-1-amine-1,1,2,2-d₄; 2-(1H-indol-3-yl)-N,N-bis(methyl-d₃)ethan-1-amine-1,1-d₂; 2-(5-methoxy-1H-indol-3-yl)-N,N-bis(methyl-d₃)ethan-1-amine-1,1,2,2-d₄; 2-(5-(methoxy-d₃)-1H-indol-3-yl)-N,N-dimethylethan-1-amine-1,1-d₂; and 2-(5-(methoxy-d₃)-1H-indol-3-yl)-N,N-bis(methyl-d₃)ethan-1-amine-1,1,2,2-d₄; or a pharmaceutically acceptable salt, solvate, or prodrug thereof.

Embodiment 31. The method of any one of Embodiments 24 to 30, wherein the psychedelic drug is a fumarate salt, benzoate salt, salicylate salt, or succinate salt of at least one selected from the group consisting of 2-(1H-indol-3-yl)-N,N-bis(methyl-d₃)ethan-1-amine-1,1,2,2-d₄; 2-(1H-indol-3-yl)-N,N-bis(methyl-d₃)ethan-1-amine-1,1-d₂; 2-(5-methoxy-1H-indol-3-yl)-N,N-bis(methyl-d₃)ethan-1-amine-1,1,2,2-d₄; 2-(5-(methoxy-d₃)-1H-indol-3-yl)-N,N-dimethylethan-1-amine-1,1-d₂; and 2-(5-(methoxy-d₃)-1H-indol-3-yl)-N,N-bis(methyl-d₃)ethan-1-amine-1,1,2,2-d₄.

Embodiment 32. The method of any one of Embodiments 24 to 31, wherein the psychedelic drug is a psychedelic drug mixture of at least two compounds of Formula (I), the psychedelic drug mixture comprising (i) 2-(1H-indol-3-yl)-N,N-bis(methyl-d₃)ethan-1-amine-1,1,2,2-d₄, or a pharmaceutically acceptable salt, solvate, or prodrug thereof; (ii) one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof.

Embodiment 33. The method of Embodiment 32, wherein the psychedelic drug mixture comprises (i) from 60% to 99% by weight of 2-(1H-indol-3-yl)-N,N-bis(methyl-d₃)ethan-1-amine-1,1,2,2-d₄, or a pharmaceutically acceptable salt, solvate, or prodrug thereof, based on a total weight of the psychedelic drug mixture; (ii) from 1% to 40% by weight, in sum, of one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof, based on a total weight of the psychedelic drug mixture; and (iii) from 0% by weight to less than 10% by weight, in sum, of one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof, based on a total weight of the psychedelic drug mixture.

Embodiment 34. The method of any one of Embodiments 24 to 33, wherein the carrier is air, oxygen, or a mixture of helium and oxygen.

Embodiment 35. The method of Embodiment 34, wherein the carrier is the mixture of helium and oxygen.

Embodiment 36. The method of Embodiment 35, wherein the mixture of helium and oxygen is heated to about 50° C. to about 60° C.

Embodiment 37. The method of Embodiment 35 or 36, wherein the helium is present in the mixture of helium and oxygen at about 50 to 90% by volume, and the oxygen is present in the mixture of helium and oxygen at about 10 to 50% by volume.

Embodiment 38. The method of any one of Embodiments 24 to 37, further comprising administering a pretreatment inhalation therapy prior to administration of the aerosol comprising the psychedelic drug and the carrier.

Embodiment 39. The method of Embodiment 38, wherein the pretreatment inhalation therapy comprises administering via inhalation a mixture of helium and oxygen heated to about 90° C. to about 120° C. to the patient.

Embodiment 40. The method of any one of Embodiments 24 to 39, wherein the psychedelic drug is delivered to the patient's central nervous system, providing an improvement in drug bioavailability by at least 25% as compared to oral delivery, increased Cₘₐₓ by at least 25% as compared to oral delivery, reduced Tₘₐₓ by at least 50% as compared to oral delivery, or a combination thereof.

Embodiment 41. The method of any one of Embodiments 24 to 40, wherein the aerosol is a mist.

Embodiment 42. The method of any one of Embodiments 24 to 41, wherein the aerosol is prepared by nebulization of the psychedelic drug.

Embodiment 43. The method of Embodiment 42, wherein the nebulization is performed with a device selected from the group consisting of a jet nebulizer, an ultrasonic nebulizer, a breath-actuated nebulizer, and a vibrating mesh nebulizer.

Embodiment 44. The method of Embodiment 42 or 43, wherein the nebulization is performed using nitrous oxide as a driving gas for entrainment of the psychedelic drug in nebulized form.

Embodiment 45. The method of Embodiment 44, wherein the nitrous oxide is present in a concentration of 15 to 25% of a volume of gas used.

Embodiment 46. The method of any one of Embodiments 24 to 45, wherein the aerosol is administered for 20 to 60 minutes.

Embodiment 47. The method of any one of Embodiments 24 to 46, wherein the CNS disorder or psychological disorder is at least one selected from the group consisting of melancholic depression, atypical depression, dysthymia, anxiety disorder, obsessive compulsive disorder, addiction disorder, alcohol use disorder, opioid use disorder, amphetamine use disorder, nicotine use disorder, cocaine use disorder, post-traumatic stress disorder (PTSD), major depressive disorder (MDD), treatment-resistant depression (TRD), suicidal ideation and suicide attempts, bipolar I disorder, bipolar II disorder, cyclothymic disorder, obsessive-compulsive disorder (OCD), generalized anxiety disorder (GAD), social anxiety disorder, Alzheimer's disease, cluster headache, migraine headaches, attention deficit hyperactivity disorder (ADHD), pain and neuropathic pain, aphantasia, childhood-onset fluency disorder, major neurocognitive disorder, mild neurocognitive disorder, sexual dysfunction, gambling disorder, eating disorder, anorexia nervosa, bulimia nervosa, binge-eating disorder, paraphilic disorders, pedophilic disorder, exhibitionistic disorder, voyeuristic disorder, fetishistic disorder, sexual masochism disorder, sexual sadism disorder, and transvestic disorder.

Embodiment 48. The method of any one of Embodiments 24 to 47, wherein the CNS disorder or a psychiatric disease is major depressive disorder (MDD).

Embodiment 49. The method of any one of Embodiments 24 to 47, wherein the CNS disorder or a psychiatric disease is treatment-resistant depression (TRD).

Embodiment 50. The method of any one of Embodiments 24 to 47, wherein the CNS disorder or a psychiatric disease is generalized anxiety disorder (GAD).

Embodiment 51. The method of any one of Embodiments 24 to 47, wherein the CNS disorder or a psychiatric disease is generalized anxiety disorder (GAD) with depression.

Embodiment 52. The method of any one of Embodiments 24 to 47, wherein the CNS disorder or a psychiatric disease is social anxiety disorder.

Embodiment 53. The method of any one of Embodiments 24 to 47, wherein the CNS disorder or a psychiatric disease is alcohol use disorder.

Embodiment 54. A method of delivering a psychedelic drug to a patient in need thereof, comprising administering a dry powder to the patient by inhalation via a dry powder inhaler, wherein the dry powder comprises the psychedelic drug, and the psychedelic drug is a compound of Formula (I) or a pharmaceutically acceptable salt, stereoisomer, solvate, or prodrug thereof

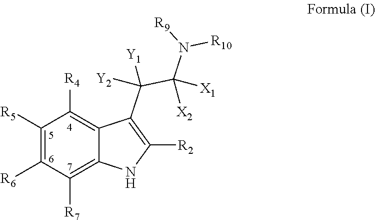

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

Embodiment 55. The method of Embodiment 54, wherein the psychedelic drug is a psychedelic drug mixture of at least two compounds of Formula (I), the psychedelic drug mixture comprising (i) 2-(1H-indol-3-yl)-N,N-bis(methyl-d₃)ethan-1-amine-1,1,2,2-d₄, or a pharmaceutically acceptable salt, solvate, or prodrug thereof; (ii) one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof.

Embodiment 56. The method of Embodiment 55, wherein the psychedelic drug mixture comprises (i) from 60% to 99% by weight of 2-(1H-indol-3-yl)-N,N-bis(methyl-d₃)ethan-1-amine-1,1,2,2-d₄, or a pharmaceutically acceptable salt, solvate, or prodrug thereof, based on a total weight of the psychedelic drug mixture; (ii) from 1% to 40% by weight, in sum, of one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof, based on a total weight of the psychedelic drug mixture; and (iii) from 0% by weight to less than 10% by weight, in sum, of one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof, based on a total weight of the psychedelic drug mixture.

Embodiment 57. The method of any one of Embodiments 54 to 56, wherein the dry powder comprises a particulate carrier having the psychedelic drug on a surface thereof.

Embodiment 58. The method of any one of Embodiments 54 to 57, wherein the psychedelic drug is releasably absorbed onto a surface of the particulate carrier, such that upon inhalation by the patient, the psychedelic drug is released from the particulate carrier within the patient.

Embodiment 59. Use of a psychedelic drug such as a compound of Formula (I) for treating a patient with a central nervous system (CNS) disorder or psychological disorder.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIGS. 1A-1B show a directed flow exposure chamber housed within a secondary containment chamber (top view; FIG. 1A) and a depiction of rats held in restraining tubes with their snouts protruding from the ends of the restraining tubes into the exposure chambers (FIG. 1B);

FIG. 2 shows DMT and DMT-d₁₀ plasma concentration-time profiles after i.v. administration (1 mg/kg) in rats;

FIG. 3 shows DMT and DMT-d₁₀ plasma concentration-time profiles after inhalation administration (14.7 mg/kg and 15.3 mg/kg, respectively) in rats;

FIG. 4 shows DMT and DMT-d₁₀ plasma concentration-time profiles after PO (oral gavage; OG) administration (10 mg/kg) in rats;

FIG. 5 shows DMT plasma concentration-time profiles after i.v., inhalation, and PO (OG) administration, with doses normalized to 1 mg/kg;

FIG. 6 shows DMT-d₁₀ plasma concentration-time profiles after i.v., inhalation, and PO (OG) administration, with doses normalized to 1 mg/kg;

### DETAILED DESCRIPTION

The following detailed description is merely exemplary in nature and is not intended to limit the described compositions or methods.

Unless defined otherwise, all technical and scientific terms used herein have the same meaning as is commonly understood by one of skill in the art to which this disclosure belongs.

“Alkyl” refers to monovalent saturated aliphatic hydrocarbyl groups having from 1 to 10 carbon atoms and such as 1 to 6 carbon atoms, or 1 to 5, or 1 to 4, or 1 to 3, or 1 to 2 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl (t-Bu)((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 10 substituents selected from the group consisting of deuterium, alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR′R″, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups having from 1 to 6, including, for example, 1 to 3 carbon atoms that are either straight-chained or branched, and which are optionally interrupted with one or more groups selected from —O—, —NR¹⁰—, —NR¹⁰C(O), —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R′ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms, for example 2 to 4 carbon atoms and having at least 1, for example from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms, for example, 2 to 3 carbon atoms and having at least 1 and for example, from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO— substituted alkyl, —SO-aryl, —SO-heteroaryl, —SO₂-alkyl, —SO₂-substituted alkyl, —SO₂-aryl, —SO₂— heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O— alkynyl, —O—C(O)O-substituted alkynyl, —O—C(O)O-aryl, —O—C(O)O-substituted aryl, —O—C(O)O— cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclohexyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from deuterium, alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and for example, from 1 to 2 double bonds.

The term “substituted cycloalkenyl” refers to cycloalkenyl groups having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In some embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In some embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, benzo[d][1,3]oxathiole, benzo[d][1,3]dioxole, and the like.

Unless otherwise constrained by the definition for the heterocyclic substituent, such heterocyclic groups can be optionally substituted with 1 to 5, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (=S).

“Alkylthio” or the term “thioalkoxy” refers to the group —S-alkyl, wherein alkyl is as defined herein. In some embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, deuterium, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂-M*, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the disclosure and the other a typical counter ion such as chloride, or two ionized compounds disclosed herein can serve as counter ions for such divalent alkali earth ions, or a doubly ionized compound of the disclosure can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, deuterium, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in some embodiments, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein, unless specified otherwise. In such cases, the maximum number of such substitutions is three. For example, serial substitutions of substituted aryl groups specifically contemplated herein are limited to substituted aryl-(substituted aryl)-substituted aryl. However, substituent groups defined as e.g., polyethers may contain serial substitution greater than three, e.g., —O—(CH₂CH₂O)ₙ—H, where n can be 1, 2, 3, or greater.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

When it is stated that a substituent or group “comprise(s) deuterium,” it is to be understood that the substituent or group may itself be deuterium, or the substituent or group may contain at least one deuterium substitution in its chemical structure. For example, when substituent “-R” is defined to “comprise(s) deuterium,” it is to be understood that -R may be -D (-deuterium), or a group such as —CD₃ that is consistent with the other requirements set forth of -R.

As used herein, the term “fatty” describes a compound with a long-chain (linear) hydrophobic portion made up of hydrogen and anywhere from 4 to 26 carbon atoms, which may be fully saturated or partially unsaturated.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, ammonium, and tetraalkylammonium salts, and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, oxalate, fumarate, benzoate, salicylate, succinate, oxalate, glycolate, hemi-oxalate, hemi-fumarate, propionate, stearate, lactate, citrate, ascorbate, pamoate, hydroxymaleate, phenylacetate, glutamate, 2-acetoxybenzoate, tosylate, ethanedisulfonate, isethionate salts, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers. All forms such as racemates and optically pure stereoisomers of the compounds are contemplated herein. Chemical formulas and compounds which possess at least one stereogenic center, but are drawn without reference to stereochemistry, are intended to encompass both the racemic compound, as well as the separate stereoisomers, e.g., R- and/or S-stereoisomers, each permutation of diastereomers so long as those diastereomers are geometrically feasible, etc.

A “crystalline” solid is a type of solid whose fundamental three-dimensional structure contains a highly regular pattern of atoms or molecules—with long range order—forming a crystal lattice, and thus displays sharp characteristic crystalline peak(s) in its X-ray power diffraction (XRPD) pattern. In some instances, crystalline solids can exist in different crystalline forms known as “polymorphs,” which have the same chemical composition, but differ in packing, geometric arrangement, and other descriptive properties of the crystalline solid state. As such, polymorphs may have different solid-state physical properties to affect, for example, the solubility, dissolution rate, bioavailability, chemical and physical stability, flowability, and compressibility, etc. of the compound as well as the safety and efficacy of drug products based on the compound. In the process of preparing a polymorph, further purification, in terms of gross physical purity or optical purity, may be accomplished as well. As used herein, the term “amorphous” refers to a solid material having substantially no long range order in the position of its molecules—the molecules are arranged in a random manner so that there is effectively no well-defined arrangement, e.g., molecular packing, and no long range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity—less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of a known standard or an internal standard. Other characterization techniques, such as differential scanning calorimetry (DSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

When referencing X-ray powder diffraction (XRPD) patterns of materials of the present disclosure, the phrase “characterized by an X-ray powder diffraction pattern containing at least three characteristic peaks at diffraction angles (2θ±0.2°) selected from . . . ” should be understood to include those materials characterized as having 3, 4, 5, 6, 7, 8, 9, 10, 11, 12, 13, 14, 15 or more (including all) of the recited characteristic XRPD diffraction peaks. Further, this phrase is intended to be open to the inclusion of other XRPD diffraction peaks not recited. Unless stated otherwise, the XRPD analyses were conducted on an X-ray powder diffractometer using a CuKα radiation source (wavelength=1.54060 Å).

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible. For example, compounds containing an acid and a base group within the same molecule depicted in neutral form may exist also in a zwitterionic form, as is the case for amino acid/ammonium carboxylate tautomers. Thus, compounds of the present disclosure which are depicted to contain both amino and dihydrogen phosphate functionality in neutral form may also exist in zwitterionic form as the ammonium monohydrogen phosphate zwitterion.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, e.g., an ester, a phosphate ester, etc. but is converted in vivo to an active compound, for example, by hydrolysis to a free carboxylic acid or free hydroxyl group. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxyl, free amino or free mercapto group, respectively. Examples of prodrugs include, but are not limited to, ester (e.g., acetate, formate, benzoate, etc.), carbonate, carbamate, and dihydrogen phosphate derivatives of an alcohol, or amide (e.g., acetamide, formamide, benzamide, etc.), carbamate, etc. derivatives of an amine functional group in the active compound, and the like.

It will be appreciated that the compounds herein can exist in different salt, solvate, and stereoisomer forms, and the present disclosure is intended to include all permutations of salts, solvates and stereoisomers, such as a solvate of a pharmaceutically acceptable salt of a stereoisomer of subject compound.

A “vapor” is a solid substance in the gas phase at a temperature lower than its critical temperature, meaning that the vapor can be condensed to a liquid by increasing the pressure on it without reducing the temperature.

An “aerosol”, as used herein, is a suspension of fine solid particles or liquid droplets in a gas phase (e.g., air, oxygen, helium, nitrous oxide, xenon, argon, and other gases, as well as mixtures thereof). A “mist”, as used herein, is a subset of aerosols, differing from a vapor, and is a dispersion of liquid droplets (liquid phase) suspended in the gas phase (e.g., air, oxygen, helium, and mixtures thereof). The liquid droplets of an aerosol or mist can comprise a drug moiety dissolved in an aqueous liquid, organic solvent, or a mixture thereof. The gas phase of an aerosol or mist can comprise air, oxygen, helium, or other gases such as nitrous oxide, xenon, and/or argon, including mixtures thereof. Mists do not comprise solid particulates. Aerosols and mists of the present disclosure can be generated by any suitable methods and devices, examples of which are set forth herein, e.g., through use of an inhaler or nebulizer.

As used herein, the term “composition” is equivalent to the term “formulation.”

As used herein, the term “inhalation session” describes a dosing event whereby the subject inhales a given dose of drug, irrespective of the number of breadths needed to inhale the given dose. For example, a subject prescribed to take 10 mg of a drug twice a day would undertake two inhalation sessions, each inhalation session providing 10 mg of the drug. The length of time and the number of breaths for each inhalation session would be dependent on factors such as the inhalation device used, the amount of drug that is drawn per breath, the concentration of the drug in the dosage form, the subject's breathing pattern, etc.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating a symptom of the disease or medical condition in a patient. A treatment can provide a therapeutic benefit such as the eradication or amelioration of one or more of the physiological or psychological symptoms associated with the underlying condition, disease, or disorder such that an improvement is observed in the patient, notwithstanding the fact that the patient may still be affected by the condition. In some embodiments, treatment may refer to prophylaxis, i.e., preventing the disease or medical condition from occurring or otherwise delaying the onset of the disease or medical condition in a patient.

A “patient” or “subject,” used interchangeably herein, can be any mammal including, for example, a human. A patient or subject can have a condition to be treated or can be susceptible to a condition to be treated.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

All diseases and disorders listed herein may be defined as described in the Diagnostic and Statistical Manual of Mental Disorders (DSM-5), published by the American Psychiatric Association, or in International Classification of Diseases (ICD), published by the World Health Organization.

In some embodiments, the present disclosure provides a method of delivering a psychedelic drug to a patient in need thereof comprising administering the psychedelic drug dissolved in an aerosol, such as a mist, via inhalation. In some embodiments, the aerosol is generated without externally added heat (this does not exclude minor temperature increases caused by the formation of the aerosol itself, such as with a vibrating mesh or other nebulizer. However, such minor temperature increases can often be offset by vaporization of the drug, which results in cooling of the composition).

The psychedelic drug can be a 5-HT_{2A} receptor agonist, including both partial and full agonists. The psychedelic drug can be any desired drug providing psychedelic effects, including, but not limited to, i) phenethylamine derivatives including, but not limited to, 3,4-methylenedioxymethamphetamine (MDMA); 2C-X phenethylamines such as 2,5-dimethoxy-4-bromophenethylamine (2C-B), (4-chloro-2,5-dimethoxyphenethyl)amine (2C-C), 2,5-dimethoxy-4-methylphenethylamine (2C-D); 3,4-methylenedioxy-N-ethylamphetamine (MDEA); 1,3-benzodioxolyl-N-methylbutanamine (MBDB); trimethoxyamphetamines (TMAs) such as 3,4,5-trimethoxyamphetamine (TMA), 2,4,5-trimethoxy-amphetamine (TMA-2), 2,3,4-trimethoxyamphetamine (TMA-3), 2,3,5-trimethoxyamphetamine (TMA-4), 2,3,6-trimethoxyamphetamine (TMA-5), and 2,4,6-trimethoxyamphetamine (TMA-6); trimethoxyphenethylamines such as 3,4,5-trimethoxyphenethylamine (mescaline) and isomescaline (2,3,4-trimethoxyphenethylamine); 2,5-dimethoxy-4-methylamphetamine (DOM); 2,5-dimethoxy-4-ethylamphetamine (DOET); 1-(2,5-dimethoxyphenyl)-2-aminopropane; 2,5-dimethoxy-4-iodoamphetamine (DOI), including (R)-DOI; 4-chloro-2,5-dimethoxy-amphetamine (DOC); 4-bromo-2,5-dimethoxy-amphetamine (DOB); 4-bromo-2,5-dimethoxy-methamphetamine (MDOB); and 4-bromo-3,6-dimethoxybenzocyclobuten-1-yl) methylamine (2C-BCB); or deuterated analogs thereof; as well as pharmaceutically acceptable salts, solvates, stereoisomers, or prodrugs thereof; (ii) tryptamine derivatives, including, but not limited to, psilocybin (3-[2-(dimethylamino)ethyl]-1H-indol-4-yl dihydrogen phosphate) and derivatives thereof, e.g., psilocin (4-hydroxy-N,N-dimethyltryptamine), N-desmethyl-psilocybin (3-[2-(methylamino)ethyl]-1H-indol-4-yl dihydrogen phosphate), 4-HO-NMT (4-hydroxy-N-methyltryptamine), norbaeocystin ([3-(2-aminoethyl)-1H-indol-4-yl]dihydrogen phosphate, 4-hydroxytryptamine, 3-[2-(N,N,N-trimethylamino)ethyl]-1H-indol-4-yl dihydrogen phosphate salts, and 4-hydroxy TMT salts (salts of 4-hydroxy-N,N,N-trimethyltryptamine); N,N-dimethyltryptamine (DMT, also referred to herein as 2-(1H-indol-3-yl)-N,N-dimethylethan-1-amine); 5-hydroxy-N,N-dimethyltryptamine (5-OH-DMT); 5-methoxy-N,N-dimethyltryptamine (5-MeO-DMT); ibogaine (a complex tryptamine); or deuterated analogs thereof, e.g., 2-(1H-indol-3-yl)-N,N-bis(methyl-d₃)ethan-1-amine-1,1,2,2-d₄ (DMT-d₁₀), 2-(1H-indol-3-yl)-N,N-bis(methyl-d₃)ethan-1-amine-1,1-d₂ (DMT-d₈), 2-(5-methoxy-1H-indol-3-yl)-N,N-bis(methyl-d₃)ethan-1-amine-1,1,2,2-d₄ (5-MeO-DMT-d₁₀), 2-(5-(methoxy-d₃)-1H-indol-3-yl)-N,N-dimethylethan-1-amine-1,1-d₂ (5-MeO-DMT-d₅), 2-(5-(methoxy-d₃)-1H-indol-3-yl)-N,N-bis(methyl-d₃)ethan-1-amine-1,1,2,2-d₄ (5-MeO-DMT-d₁₃), psilocin-d₁₀ (3-(2-(bis(methyl-d₃)amino)ethyl-1,1,2,2-d₄)-1H-indol-4-ol), N,N-bis(methyl-d₃)-2-(5-methyl-1H-indol-3-yl)ethan-1-amine-1,1,2,2-d₄, N,N-bis(methyl-d₃)-2-(5-(methyl-d₃)-1H-indol-3-yl)ethan-1-amine-1,1,2,2-d₄, etc., as well as pharmaceutically acceptable salts, solvates, stereoisomers, or prodrugs thereof; (iii) lysergamides such as lysergic acid diethylamide (LSD) and derivatives thereof, e.g., LA-SS-Az (“LSZ” or (2S,4S)-1-[[(8β)-9,10-Didehydro-6-(methyl)ergolin-8-yl]carbonyl]-2,4-dimethylazetidine); or deuterated analogs thereof; as well as pharmaceutically acceptable salts, solvates, or stereoisomers thereof; including any combinations thereof.

In some embodiments, the psychedelic drug is a compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), and Formula (VI-b) described herein, any exemplary compounds described herein, or a pharmaceutically acceptable salt, solvate, stereoisomer, or prodrug thereof, or a combination thereof. In some embodiments, the psychedelic drug can be delivered as an aerosol, such as a mist, with a carrier, such as air, oxygen, or a mixture of helium and oxygen. In some embodiments, the carrier can be a mixture of helium and oxygen heated to about 50° C. to about 60° C.

In some embodiments, the psychedelic drug comprises one or more of N,N-dimethyltryptamine (DMT), 5-hydroxy-N,N-dimethyltryptamine (5-OH-DMT), 5-methoxy-N,N-dimethyltryptamine (5-MeO-DMT), 4-hydroxy-N,N-dimethyltryptamine (psilocin), DMT-d₁₀(2-(1H-indol-3-yl)-N,N-bis(methyl-d₃)ethan-1-amine-1,1,2,2-d₄), 5-MeO-DMT-d₁₀ (2-(5-methoxy-1H-indol-3-yl)-N,N-bis(methyl-d₃)ethan-1-amine-1,1,2,2-d₄), and 3-(2-(bis(methyl-d₃)amino)ethyl-1,1,2,2-d₄)-1H-indol-4-ol (psilocin-d₁₀), or a pharmaceutically acceptable salt or solvate thereof.

In some embodiments, the psychedelic drug is N,N-dimethyltryptamine (DMT), or a pharmaceutically acceptable salt or solvate thereof.

In some embodiments, the psychedelic drug is 5-hydroxy-N,N-dimethyltryptamine (5-OH-DMT), or a pharmaceutically acceptable salt or solvate thereof.

In some embodiments, the psychedelic drug is 5-methoxy-N,N-dimethyltryptamine (5-MeO-DMT), or a pharmaceutically acceptable salt or solvate thereof.

In some embodiments, the psychedelic drug comprises a deuterated tryptamine. In some embodiments, the psychedelic drug is 2-(1H-indol-3-yl)-N,N-bis(methyl-d₃)ethan-1-amine-1,1,2,2-d₄ (DMT-d₁₀), or a pharmaceutically acceptable salt or solvate thereof. In some embodiments, the psychedelic drug is 2-(1H-indol-3-yl)-N,N-bis(methyl-d₃)ethan-1-amine-1,1-d₂ (DMT-d₈), or a pharmaceutically acceptable salt or solvate thereof. In some embodiments, the psychedelic drug is 2-(5-methoxy-1H-indol-3-yl)-N,N-bis(methyl-d₃)ethan-1-amine-1,1,2,2-d₄ (5-MeO-DMT-d₁₀), or a pharmaceutically acceptable salt or solvate thereof. In some embodiments, the psychedelic drug is 2-(5-(methoxy-d₃)-1H-indol-3-yl)-N,N-dimethylethan-1-amine-1,1-d₂ (5-MeO-DMT-d₅), or a pharmaceutically acceptable salt or solvate thereof. In some embodiments, the psychedelic drug is 2-(5-(methoxy-d₃)-1H-indol-3-yl)-N,N-bis(methyl-d₃)ethan-1-amine-1,1,2,2-d₄ (5-MeO-DMT-d₁₃), or a pharmaceutically acceptable salt or solvate thereof. In some embodiments, the psychedelic drug is 3-(2-(bis(methyl-d₃)amino)ethyl-1,1,2,2-d₄)-1H-indol-4-ol (psilocin-d₁₀), or a pharmaceutically acceptable salt or solvate thereof. In some embodiments, the psychedelic drug is N,N-bis(methyl-d₃)-2-(5-methyl-1H-indol-3-yl)ethan-1-amine-1,1,2,2-d₄, or a pharmaceutically acceptable salt or solvate thereof. In some embodiments, the psychedelic drug is N,N-bis(methyl-d₃)-2-(5-(methyl-d₃)-1H-indol-3-yl)ethan-1-amine-1,1,2,2-d₄, or a pharmaceutically acceptable salt or solvate thereof.

In some embodiments, the psychedelic drug is psilocybin or derivatives thereof. In some embodiments, the psychedelic drug is psilocin, or a pharmaceutically acceptable salt or solvate thereof.

Additionally, by administration via inhalation, the psychedelic drug can be delivered systemically to the patient's central nervous system. The air, oxygen, or mixture of helium and oxygen can be heated to about 55° C. to about 56° C. When a mixture of helium and oxygen is used as the carrier, the helium can be present in the mixture of oxygen and helium at about 50%, 60%, 70%, 80% or 90% by volume and the oxygen can be present in the mixture at about 50%, 40%, 30%, 20%, or 10% by volume.

The method can further comprise administering a pretreatment inhalation therapy prior to administration of the aerosol comprising the psychedelic drug. The pretreatment can comprise administering via inhalation of a mixture of helium and oxygen heated to about 90° C. to about 120° C. (e.g., about 90, 100, 110, or 120° C.) to the patient.

The method can comprise (i) administering via inhalation a mixture of helium and oxygen heated to about 90° C. to about 120° C. (e.g., about 90, 100, 110, or 120° C.) to the patient, followed by (ii) administering via inhalation a mixture of helium and oxygen heated to about 50° C. to about 60° C. (e.g., about 50, 52, 53, 56, 58, or 60° C.) and the aerosol comprising the psychedelic drug to the patient and then repeating steps (i) and (ii). Steps (i) and (ii) can be repeated 1, 2, 3, 4, 5, or more times.

In some embodiments, the present disclosure provides a method of treating a central nervous system (CNS) disorder or psychological disorder comprising administering, via inhalation, a psychedelic drug in the form of an aerosol, such as a mist.

The psychedelic drug can be delivered as an aerosol along with a carrier such as air, oxygen, a mixture of helium and oxygen, a mixture of nitrous oxide (or noble gas such as xenon and/or argon) and oxygen or air, or other gases or gas mixtures. The mixture of helium and oxygen can be heated to about 50° C. to about 60° C. (e.g., about 50, 52, 53, 56, 58, or 60° C.) prior to administering the aerosol comprising the psychedelic drug to the patient.

The central nervous system or psychological disorder can be, for example, melancholic depression, atypical depression, dysthymia, anxiety disorder, obsessive compulsive disorder, addiction (narcotic addiction, tobacco addiction, opioid addiction), alcoholism, post-traumatic stress disorder (PTSD), major depressive disorder (MDD), treatment-resistant depression (TRD), suicidal ideation and suicide attempts, bipolar and related disorders, generalized anxiety disorder (GAD), social anxiety disorder, anorexia nervosa, bulimia nervosa, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, gambling disorder, eating disorders such as anorexia nervosa, bulimia nervosa, binge-eating disorder, etc., and paraphilic disorders such as, for example, pedophilic disorder, exhibitionistic disorder, voyeuristic disorder, fetishistic disorder, sexual masochism or sadism disorder, and transvestic disorder, etc., or other set forth herein.

In some embodiments, the psychedelic drug is delivered by inhalation to the patient's central nervous system resulting in an improvement in drug bioavailability by at least 25% (e.g., at least about 25, 30, 35, 40, 45, 50% or more) as compared to oral delivery, increased Cₘₐₓ by at least 25% (e.g., at least about 25, 30, 35, 40, 45, 50% or more) as compared to oral delivery, reduced Tₘₐₓ by at least 50% (e.g., reduced by at least 50, 60, 70, 80% or more) as compared to oral delivery, or a combination thereof.

Good aqueous solubility of most psychedelics including e.g., DMT (in the salt form) makes inhalation of an aerosol, such as a mist, a possible route of administration. However, inhalation of a mist is mainly used for local rather than systemic drug delivery and faces multiple challenges related to inconsistent and incomplete dose delivery, variability in the breathing pattern, deposition of the dose in the upper respiratory tract, etc. Methods are provided herein for mist inhalation administration of psychedelic drugs.

### Psychedelic Drugs

Therapeutic agents of the present disclosure can include any desired psychedelic drug. The term “psychedelic,” “psychedelic compound,” “psychedelic agent,” or “psychedelic drug” as used herein can encompass a number of compounds including serotonin 5-HT_{2A} receptor agonists (e.g., lysergic acid diethylamide (LSD)), empathogenic agents (i.e., serotonin (5-HT) releasing agents; e.g., 3,4-methylenedioxymethamphetamine (MDMA)), and dissociative agents (i.e., N-Methyl-D-aspartate (NMDA) receptor agonists; e.g., nitrous oxide, xenon, argon, ketamine and dextromethorphan), including pharmaceutically acceptable stereoisomers, salts, and solvates thereof; as well as combinations thereof. Most psychedelic drugs fall within the following groups: tryptamines (also referred to herein as tryptamine derivatives), phenethylamines (also referred to herein as phenethylamine derivatives), or lysergamides. These drugs all activate serotonin 5-HT_{2A} receptors, which modulate the activity of key circuits in the brain involved with sensory perception and cognition. Tryptamine derivatives include those set forth herein, for example, 5-methoxy-dimethyltryptamine (5-MeO-DMT), 5-hydroxy-dimethyltryptamine (5-OH-DMT), dimethyltryptamine (DMT), or derivatives thereof such as deuterated analogs. 5-OH-DMT is also known as bufotenine. 5-MeO-DMT is a prodrug to bufotenine via demethylation. DMT is also known as N,N-dimethyltryptamine and is a primary active constituent of ayahuasca. Other psychedelic drugs are described below.

A derivative includes any compound that is made from a psychedelic drug such as one of the psychedelic drugs described herein, for example, by replacing one atom in the psychedelic drug with another atom or group of atoms, rearranging two or more atoms in the psychedelic drug, ionizing a psychedelic drug, or creating a salt of one of the psychedelic drugs.

Unless clearly indicated to the contrary, the term “derivative” does not necessarily mean that the derivative is synthesized using the parent compound as a starting material or as an intermediate, although in some cases, the derivative can be synthesized from the parent. In some embodiments, a derivative of a psychedelic drug has therapeutic activity.

As used herein, a “5-HT_{2A} receptor agonist” refers to a compound that increases the activity of a 5-hydroxytryptamine 2A receptor, which is a subtype of the 5-HT₂ receptor that belongs to the serotonin receptor family. Examples of such agonists include, but are not limited to, those phenethylamine derivatives, tryptamine derivatives, and/or lysergamides set forth herein, for example, psilocybin and derivatives thereof (e.g., psilocin, N-desmethyl-psilocybin, 4-HO-NMT, norbaeocystin, 3-[2-(N,N,N-trimethylamino)ethyl]-1H-indol-4-yl dihydrogen phosphate salts, and 4-hydroxy TMT salts), DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer) (a substituted amphetamine); LA-SS-Az (“LSZ” or (2S,4S)-1-[[(8p)-9,10-Didehydro-6-(methyl)ergolin-8-yl]carbonyl]-2,4-dimethylazetidine) (a complex tryptamine); 2C-BCB (4-bromo-3,6-dimethoxybenzocyclobuten-1-yl) methylamine; 3,4,5-trimethoxyphenethylamine (mescaline); ibogaine; N,N-dimethyltryptamine (DMT); 5-hydroxy-N,N-dimethyltryptamine (5-OH-DMT); 5-methoxy-N,N-dimethyltryptamine (5-MeO-DMT); 2-(1H-indol-3-yl)-N,N-bis(methyl-d₃)ethan-1-amine-1,1,2,2-d₄(DMT-d₁₀); 2-(1H-indol-3-yl)-N,N-bis(methyl-d₃)ethan-1-amine-1,1-d₂ (DMT-d₈); 2-(5-methoxy-1H-indol-3-yl)-N,N-bis(methyl-d₃)ethan-1-amine-1,1,2,2-d₄ (5-MeO-DMT-d₁₀); 2-(5-(methoxy-d₃)-1H-indol-3-yl)-N,N-dimethylethan-1-amine-1,1-d₂ (5-MeO-DMT-d₅); 2-(5-(methoxy-d₃)-1H-indol-3-yl)-N,N-bis(methyl-d₃)ethan-1-amine-1,1,2,2-d₄ (5-MeO-DMT-d₁₃); psilocin-d₁₀ (3-(2-(bis(methyl-d₃)amino)ethyl-1,1,2,2-d₄)-1H-indol-4-ol); N,N-bis(methyl-d₃)-2-(5-methyl-1H-indol-3-yl)ethan-1-amine-1,1,2,2-d₄, N,N-bis(methyl-d₃)-2-(5-(methyl-d₃)-1H-indol-3-yl)ethan-1-amine-1,1,2,2-d₄; a compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), and Formula (VI-b) described herein; any exemplary compounds described herein; as well as a pharmaceutically acceptable salt, solvate, stereoisomer, or prodrug thereof; or a combination thereof.

In some embodiments, the psychedelic drug is a compound of Formula (I), or a pharmaceutically acceptable salt, stereoisomer, solvate, or prodrug thereof

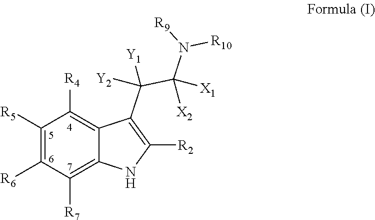

wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

X₁ and X₂ may be the same, or different. In some embodiments, X₁ and X₂ are the same. In some embodiments, X₁ and X₂ are hydrogen. In some embodiments, X₁ and X₂ are deuterium. In some embodiments, X₁ and X₂ are different. In some embodiments, X₁ is hydrogen or deuterium, and X₂ is a substituted or unsubstituted C₁-C₆ alkyl. In some embodiments, X₂ is an unsubstituted C₁-C₆ alkyl, examples of which include, but are not limited to, methyl, ethyl, and n-propyl, preferably methyl. In some embodiments, X₂ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, one of X₁ and X₂ is deuterium while the other is hydrogen. In some embodiments, one or more of X₁ and X₂ is a substituted or unsubstituted C₃-C₁₀ cycloalkyl. In some embodiments, one or more of X₁ and X₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, one or more of X₁ and X₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, X₁ and/or X₂ is an unsubstituted or substituted alkenyl, e.g., a unsubstituted or substituted allyl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

In some embodiments, R₂ is deuterium. In some embodiments, R₂ is hydrogen. In some embodiments, R₂ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₂ is a substituted C₁-C₆ alkyl. When R₂ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₂ is a substituted or unsubstituted C₃-C₁₀ cycloalkyl. In some embodiments, R₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₂ is an unsubstituted or substituted alkenyl, e.g., a unsubstituted or substituted allyl.

R₄ and R₅ may be the same, or different. In some embodiments, R₄ is deuterium. In some embodiments, R₄ is hydrogen. In some embodiments, R₄ is hydroxy. In some embodiments, R₄ is an unsubstituted C₁-C₆ alkyl, examples of which include, but are not limited to, methyl, ethyl, and n-propyl, preferably methyl. In some embodiments, R₄ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₄ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₄ is a substituted alkoxy. When R₄ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be —OCDH₂, —OCD₂H, —OCD₃, —OCFH₂, —OCF₂H, —OCF₃, etc.

In some embodiments, R₅ is deuterium. In some embodiments, R₅ is hydrogen. In some embodiments, R₅ is hydroxy. In some embodiments, R₅ is an unsubstituted C₁-C₆ alkyl, examples of which include, but are not limited to, methyl, ethyl, and n-propyl, preferably methyl. In some embodiments, R₅ is a substituted C₁-C₆ alkyl. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R₅ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₅ is a substituted alkoxy. When R₅ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be —OCDH₂, —OCD₂H, —OCD₃, —OCFH₂, —OCF₂H, —OCF₃, etc.

R₆ and R₇ may be the same, or different. R₆ and R₇ may be, independently, hydrogen, deuterium, or a halogen for example —Br, —F, —Cl, or —I.

R₉ and R₁₀ may be the same, or different. In some embodiments, R₉ and R₁₀ are the same. In some embodiments, R₉ and R₁₀ are hydrogen. In some embodiments, R₉ and R₁₀ are different. In some embodiments, R₉ is hydrogen, and R₁₀ is a substituted or unsubstituted C₁-C₆ alkyl. In some embodiments, R₉ and/or R₁₀ is an unsubstituted C₁-C₆ alkyl, examples of which include, but are not limited to, methyl, ethyl, and n-propyl, preferably methyl. In some embodiments, R₉ and/or R₁₀ is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R₉ and/or R₁₀ is a substituted or unsubstituted C₃-C₁₀ cycloalkyl. In some embodiments, R₉ and/or R₁₀ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₉ and/or R₁₀ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₉ and/or R₁₀ is an unsubstituted or substituted alkenyl, e.g., a unsubstituted or substituted allyl.

In some embodiments, the psychedelic drug is a compound of Formula (I), or a pharmaceutically acceptable salt, stereoisomer, solvate, or prodrug thereof, wherein any one or more of X₁, X₂, Y₁, Y₂, R₂, R₄, R₅, R₆, R₇, R₉, and R₁₀ optionally comprises deuterium. In some embodiments, at least one of X₁, X₂, Y₁, Y₂, R₂, R₄, R₅, R₆, R₇, R₉, and R₁₀ comprises deuterium. In some embodiments, at least one of X₁, X₂, Y₁, Y₂, R₅, R₉, and R₁₀ comprises deuterium. In some embodiments, at least one of X₁, X₂, Y₁, Y₂, R₉, and R₁₀ comprises deuterium. In some embodiments, X₁, X₂, R₉, and R₁₀ comprise deuterium. In some embodiments, X₁, X₂, Y₁, Y₂, R₉, and R₁₀ comprise deuterium. In some embodiments, X₁, X₂, and R₅ comprise deuterium. In some embodiments, X₁, X₂, Y₁, Y₂, R₅, R₉, and R₁₀ comprise deuterium.

In some embodiments, the psychedelic drug is a compound of Formula (II), or a pharmaceutically acceptable salt, stereoisomer, solvate, or prodrug thereof

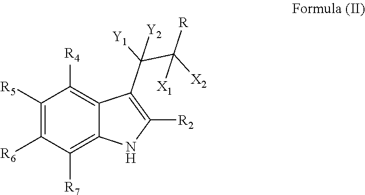

wherein:
X₁ and X₂ are deuterium;
Y₁ and Y₂ are independently selected from the group consisting of hydrogen and deuterium;
R is

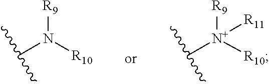

R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, unsubstituted or substituted alkoxy, and unsubstituted or substituted phosphoryloxy;
R₆ and R₇ are independently selected from the group consisting of hydrogen, deuterium, and halogen; and
R₉, R₁₀, and R₁₁ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

Y₁ and Y₂ may be the same, or different. In some embodiments, Y₁ and Y₂ are the same. In some embodiments, Y₁ and Y₂ are hydrogen. In some embodiments, Y₁ and Y₂ are deuterium. In some embodiments, Y₁ and Y₂ are different. In some embodiments, one of Y₁ and Y₂ is deuterium while the other is hydrogen.

In some embodiments, R₂ is deuterium. In some embodiments, R₂ is hydrogen. In some embodiments, R₂ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R₂ is a substituted C₁-C₆ alkyl. When R₂ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R₂ is a substituted or unsubstituted C₃-C₁₀ cycloalkyl. In some embodiments, R₂ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₂ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₂ is an unsubstituted or substituted alkenyl, e.g., a unsubstituted or substituted allyl.

R₄ and R₅ may be the same, or different. In some embodiments, R₄ is deuterium. In some embodiments, R₄ is hydrogen. In some embodiments, R₄ is hydroxy. In some embodiments, R₄ is an unsubstituted C₁-C₆ alkyl, examples of which include, but are not limited to, methyl, ethyl, and n-propyl, preferably methyl. In some embodiments, R₄ is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R₄ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₄ is a substituted alkoxy. When R₄ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be-OCDH₂, —OCD₂H, —OCD₃, —OCFH₂, —OCF₂H, —OCF₃, etc. In some embodiments, R₄ is an unsubstituted phosphoryloxy group (i.e., —OP(O)(OH)₂ or its deprotonated forms). In some embodiments, R₄ is a substituted phosphoryloxy group where one or more of the hydrogen atoms in —OP(O)(OH)₂ is replaced with a substituent group such as unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, unsubstituted or substituted heteroaryl, or other substituent group as set forth herein. When both hydrogen atoms in —OP(O)(OH)₂ are replaced with a substituent group, the substituent groups can be the same or different from one another.

In some embodiments, R₅ is deuterium. In some embodiments, R₅ is hydrogen. In some embodiments, R₅ is hydroxy. In some embodiments, R₅ is an unsubstituted C₁-C₆ alkyl, examples of which include, but are not limited to, methyl, ethyl, and n-propyl, preferably methyl. In some embodiments, R₅ is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R₅ is an unsubstituted alkoxy group, examples of which include, but are not limited to, methoxy, ethoxy, n-propoxy, isopropoxy, n-butoxy, isobutoxy, sec-butoxy, t-butoxy, n-pentoxy, neopentoxy, and hexoxy. In some embodiments, R₅ is a substituted alkoxy. When R₅ is a substituted alkoxy, preferred substituents may include, but are not limited to, deuterium, halogen (e.g., fluorine), polar substituents such as hydroxyl or polyether substituents, etc. The alkoxy group may contain one, or more than one, substituent. For example, when the alkoxy group is a C₁ alkoxy group (i.e., methoxy group), the substituted C₁ alkoxy group may be —OCDH₂, —OCD₂H, —OCD₃, —OCFH₂, —OCF₂H, —OCF₃, etc. In some embodiments, R₅ is an unsubstituted phosphoryloxy group (i.e., —OP(O)(OH)₂ or its deprotonated forms). In some embodiments, R₅ is a substituted phosphoryloxy group where one or more of the hydrogen atoms in —OP(O)(OH)₂ is replaced with a substituent group such as unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, unsubstituted or substituted heteroaryl, or other substituent group as set forth herein. When both hydrogen atoms in —OP(O)(OH)₂ are replaced with a substituent group, the substituent groups can be the same or different from one another.

R₆ and R₇ may be the same, or different. R₆ and R₇ may be, independently, hydrogen, deuterium, or a halogen for example —Br, —F, —Cl, or —I.

In some embodiments, R is

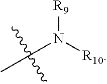

R₉ and R₁₀ may be the same, or different. In some embodiments, R₉ and R₁₀ are the same. In some embodiments, R₉ and R₁₀ are hydrogen. In some embodiments, R₉ and R₁₀ are different. In some embodiments, R₉ is hydrogen, and R₁₀ is a substituted or unsubstituted C₁-C₆ alkyl. In some embodiments, R₉ and/or R₁₀ is an unsubstituted C₁-C₆ alkyl, examples of which include, but are not limited to, methyl, ethyl, and n-propyl, preferably methyl. In some embodiments, R₉ and/or R₁₀ is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R₉ and/or R₁₀ is a substituted or unsubstituted C₃-C₁₀ cycloalkyl. In some embodiments, R₉ and/or R₁₀ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₉ and/or R₁₀ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₉ and/or R₁₀ is an unsubstituted or substituted alkenyl, e.g., a unsubstituted or substituted allyl.

In some embodiments, R is an ammonium cation represented by

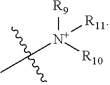

R₉ and R₁₀ are set forth above. R₉, R₁₀, and R₁₁ may be the same, or different. In some embodiments, R₉, R₁₀, and R₁₁ are the same. In some embodiments, R₉, R₁₀, and R₁₁ are each different. In some embodiments, two of R₉, R₁₀, and R₁₁ are the same. In some embodiments, Ru is hydrogen. In some embodiments, R₁₁ is an unsubstituted C₁-C₆ alkyl, examples of which include, but are not limited to, methyl, ethyl, and n-propyl, preferably methyl. In some embodiments, R₁₁ is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R₁₁ is a substituted or unsubstituted C₃-C₁₀ cycloalkyl. In some embodiments, R₁₁ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R₁₁ is a substituted C₃-C₁₀ cycloalkyl. Preferred substituents may include, but are not limited to, alkyl, deuterium, halogen (e.g., fluorine), polar substituents such as hydroxyl or polyether substituents, etc. The cycloalkyl group may contain one, or more than one, substituent. In some embodiments, R₁₁ is an unsubstituted or substituted alkenyl, e.g., a unsubstituted or substituted allyl. In some embodiments, R is a quaternary ammonium cation (where R₉, R₁₀, and R₁₁ are each not hydrogen). In some embodiments, R is a protonated ammonium cation, in which one, two, or three of R₉, R₁₀, and R₁₁ is hydrogen. When R represents either a quaternary ammonium cation or a protonated ammonium cation, R may be accompanied by a suitable conjugate base pair, examples of which include, but are not limited to, the conjugate base of any of acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, or a fatty acid (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

In some embodiments, the psychedelic drug is a compound of Formula (II-a) or a pharmaceutically acceptable salt, stereoisomer, solvate, or prodrug thereof

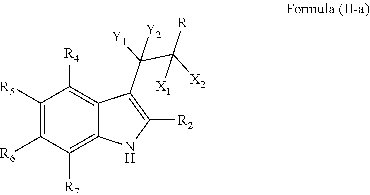

wherein:
X₁ and X₂ are deuterium;
Y₁ and Y₂ are hydrogen;
R is

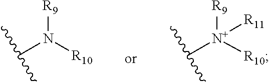

and
R₂, R₄, R₅, R₆, R₇, R₉, R₁₀, and R₁₁ are as defined above for Formula (II).

In some embodiments, the psychedelic drug is a compound of Formula (II-b) or a pharmaceutically acceptable salt, stereoisomer, solvate, or prodrug thereof.

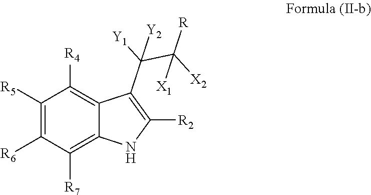

wherein:
X₁ and X₂ are deuterium;
Y₁ and Y₂ are hydrogen;
R is

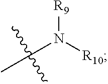

and
R₂, R₄, R₅, R₆, R₇, R₉, and R₁₀ are as defined above for Formula (II).

In some embodiments, the psychedelic drug is a compound of Formula (II-c) or a pharmaceutically acceptable salt, stereoisomer, solvate, or prodrug thereof

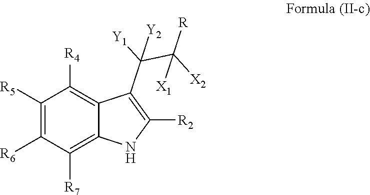

wherein:
X₁ and X₂ are deuterium; Y₁ and Y₂ are hydrogen;
R is

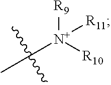

and
R₂, R₄, R₅, R₆, R₇, R₉, R₁₀, and R₁₁ are as defined above for Formula (II).

In some embodiments, the psychedelic drug is a compound of Formula (II-d) or a pharmaceutically acceptable salt, stereoisomer, solvate, or prodrug thereof

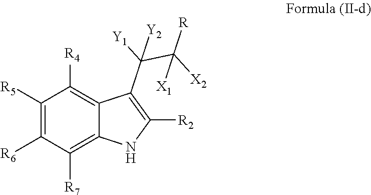

wherein:
X₁ and X₂ are deuterium;
Y₁ and Y₂ are hydrogen;
R is

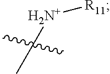

and
R₂, R₄, R₅, R₆, R₇, and R₁₁ are as defined above for Formula (II).

In some embodiments, the psychedelic drug is at least one compound selected from the group consisting of

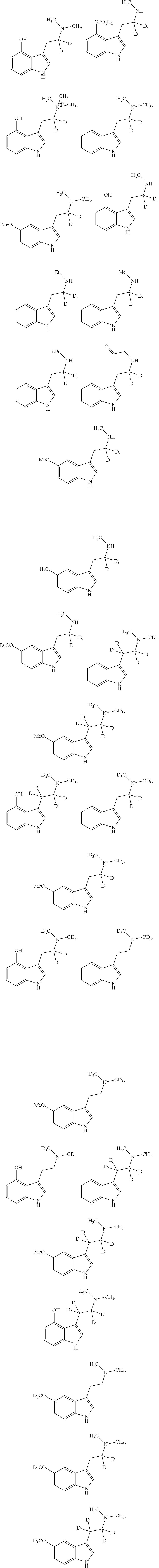

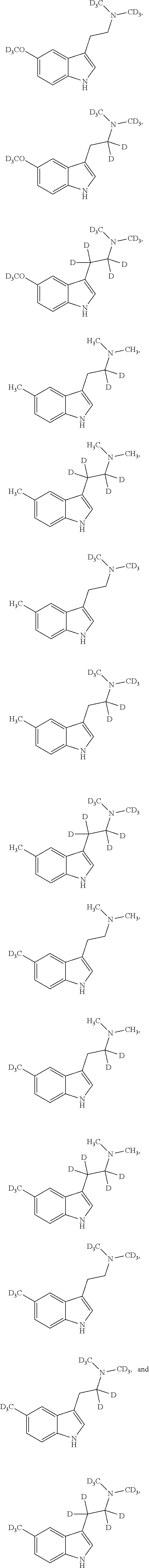

or a pharmaceutically acceptable salt, solvate, or prodrug thereof.

In some embodiments, the psychedelic drug is at least one compound selected from the group consisting of

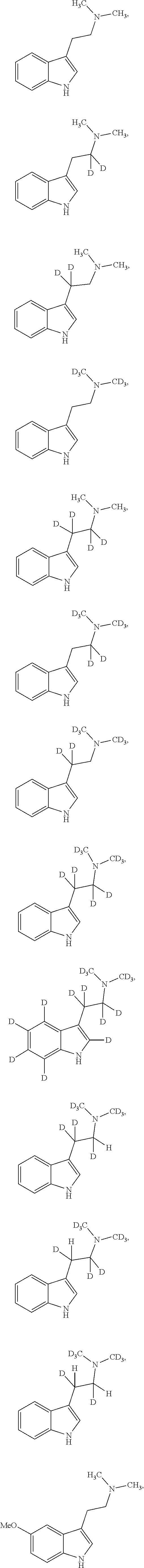

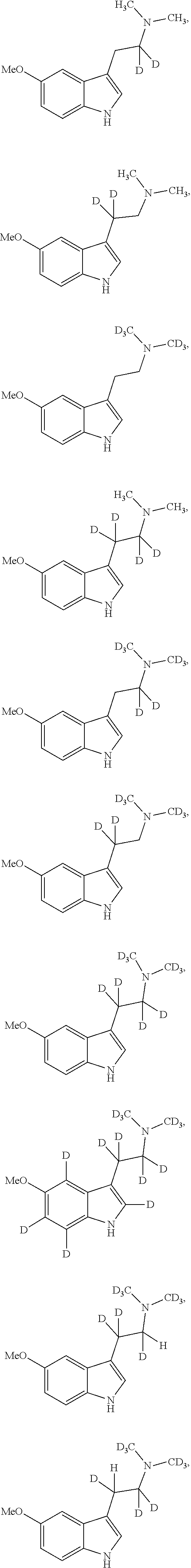

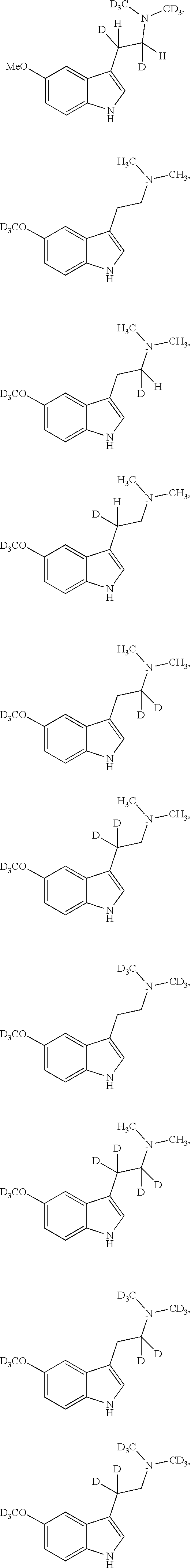

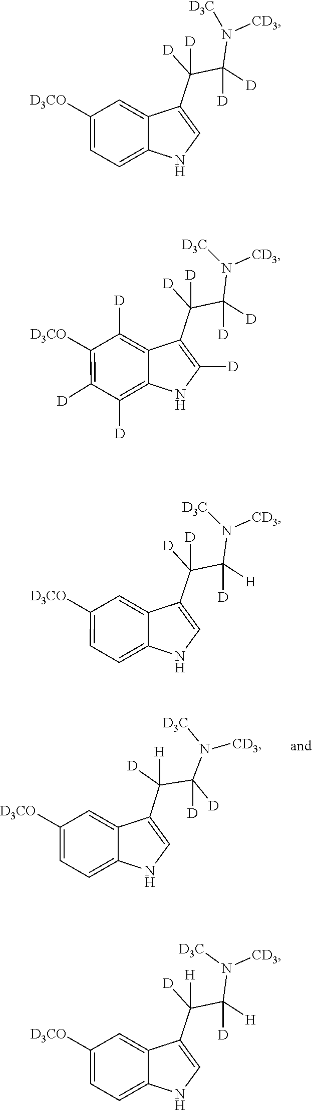

or a pharmaceutically acceptable salt, stereoisomer, solvate, or prodrug thereof.

In some embodiments, the psychedelic drug is a compound of Formula (II), Formula (II-a), Formula (II-b), Formula (II-c), and/or Formula (II-d), wherein at least one of R₂, R₄, R₅, R₆, and R₇ comprises deuterium.

In some embodiments, R₆ and/or R₇ of the compounds described herein, e.g., compounds of Formula (II), Formula (II-a), Formula (II-b), Formula (II-c), and Formula (II-d), is halogen.

In some embodiments, R₄ and/or R₅ of the compounds described herein, e.g., compounds of Formula (II), Formula (II-a), Formula (II-b), Formula (II-c), and Formula (II-d), comprise(s) deuterium.

In some embodiments, X₁, X₂, Y₁, Y₂, R₉, and R₁₀ comprise deuterium. In some embodiments, the psychedelic drug is at least one compound selected from the group consisting of

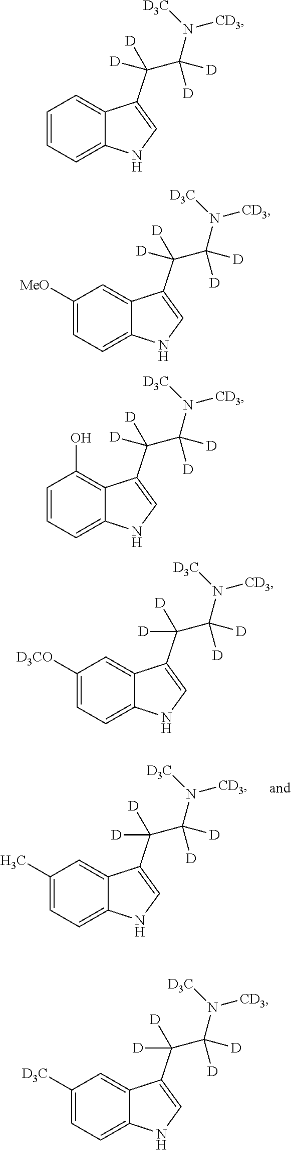

or a pharmaceutically acceptable salt, solvate, or prodrug thereof.

In some embodiments, the psychedelic drug is a compound of Formula (III) or a pharmaceutically acceptable salt, stereoisomer, solvate, or prodrug thereof

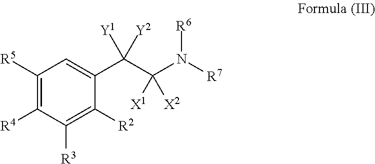

wherein:
X¹ and X² are independently selected from the group consisting of hydrogen, deuterium, and unsubstituted or substituted C₁-C₆ alkyl;
Y¹ and Y² are independently selected from the group consisting of hydrogen and deuterium;
R² and R³ are independently selected from the group consisting of hydrogen, deuterium, halogen, unsubstituted or substituted C₁-C₆ alkyl, and —OR^{a};
R⁴ and R⁵ are independently selected from the group consisting of hydrogen, deuterium, halogen, a substituted or unsubstituted C₁-C₆ alkyl, —OR^{a}, and —SR^{a}, or R⁴ and R⁵ together with the atoms to which they are attached optionally form an unsubstituted or substituted heterocycloalkyl or an unsubstituted or substituted heteroaryl;
R⁶ and R⁷ are independently selected from the group consisting of hydrogen and unsubstituted or substituted C₁-C₆ alkyl; and
each R^{a} is independently selected from the group consisting of hydrogen, deuterium, and unsubstituted or substituted C₁-C₆ alkyl.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of X¹ and X² is deuterium while the other is hydrogen.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, X¹ and X² are different. In some embodiments, one of Y¹ and Y² is deuterium while the other is hydrogen.

In some embodiments, R² is deuterium. In some embodiments, R² is hydrogen. In some embodiments, R² is halogen, for example —Br, —F, —Cl, or —I. In some embodiments, R² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R² is a substituted C₁-C₆ alkyl. When R² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R² is —OR^{a}.

In some embodiments, R³ is deuterium. In some embodiments, R³ is hydrogen. In some embodiments, R³ is halogen, for example —Br, —F, —Cl, or —I. In some embodiments, R³ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R³ is a substituted C₁-C₆ alkyl. When R³ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R³ is —OR^{a}.

In some embodiments, R⁴ is deuterium. In some embodiments, R⁴ is hydrogen. In some embodiments, R⁴ is halogen, for example —Br, —F, —Cl, or —I. In some embodiments, R⁴ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁴ is —OR^{a}. In some embodiments, R⁴ is —SR^{a}. In some embodiments, R⁴ is —SMe, —SCD₃, —SCF₃,-SEt, —Sn—Pr, —SCH₂CH₂CF₃, —SCH₂CH₂CF₂H, —SCH₂CH₂CFH₂,-Me, —CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR^{a}, and R^{a} is C₁-C₆ alkyl, which is unsubstituted or substituted with one or more deuteriums.

In some embodiments, R⁵ is deuterium. In some embodiments, R⁵ is hydrogen. In some embodiments, R⁵ is halogen, for example —Br, —F, —Cl, or —I. In some embodiments, R⁵ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁵ is a substituted C₁-C₆ alkyl. When R⁵ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁵ is —OR^{a}. In some embodiments, R⁵ is —SR^{a}. In some embodiments, R⁵ is hydrogen, —OMe, or —OCD₃. In some embodiments, R⁵ is hydrogen. In some embodiments, R⁵ is —OMe. In some embodiments, R⁵ is —OCD₃. In some embodiments, R⁵ is hydrogen, deuterium, halogen, —OR^{a}, or —SR^{a}, and R^{a} is C₁-C₆ alkyl, which is unsubstituted or substituted with one or more deuteriums. In some embodiments, R⁴ is —OCH₃, —OCD₃, —Br, —SCH₃, —SCH₂CH₃, or —SCH₂CH₂CH₃, and/or R⁵ is hydrogen, —OMe, or —OCD₃.

In some embodiments, R⁴ and R⁵ together with the atoms attached thereto are joined to form a heterocycloalkyl or heteroaryl, with specific mention being made to a benzo[d][1,3]oxathiole group or a benzo[d][1,3]dioxole group. In embodiments where R⁴ and R⁵ together with the atoms attached thereto are joined to form a heterocycloalkyl or heteroaryl (e.g., benzo[d][1,3]oxathiole group, a benzo[d][1,3]dioxole group, etc.), the heterocycloalkyl or heteroaryl ring (e.g., oxathiole ring, the dioxole ring, etc.) may be further substituted with substituents as defined herein, e.g., with one or more halogen (e.g., fluorine) or deuterium substituents.

R⁶ and R⁷ may be the same, or different. R⁶ and R⁷ may be, independently, hydrogen, an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more deuterium (e.g., —CDH₂, —CD₂H, —CD₃).

Each R^{a} may be, independently, hydrogen, deuterium, an unsubstituted C₁-C₆ alkyl (e.g., methyl, ethyl, n-propyl, isopropyl, n-butyl, isobutyl, sec-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, more than one R^{a} is present. In such cases, each R^{a} may be the same, or different. In some embodiments, each R^{a} is the same. In some embodiments, each R^{a} is different, e.g., one R^{a} is —CH₃, while another is —CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂CH₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of X¹, X², Y¹, Y², R², R³, R⁴, R⁵, R⁶, and R⁷ comprises deuterium.

In some embodiments, the psychedelic drug is a compound of Formula (III-a) or a pharmaceutically acceptable salt, stereoisomer, solvate, or prodrug thereof

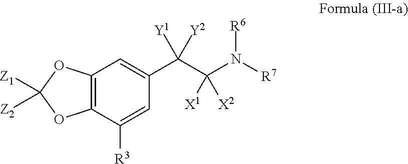

wherein:
Z¹ and Z² are independently selected from the group consisting of hydrogen, deuterium, or fluorine; and
X¹, X², Y¹, Y², R³, R⁶, R⁷, and R^{a} are as defined for Formula (III).

Z¹ and Z² may be the same, or different. In some embodiments, Z¹ and Z² are the same. In some embodiments, Z¹ and Z² are hydrogen. In some embodiments, Z¹ and Z² are deuterium. In some embodiments, Z¹ and Z² are fluorine. In some embodiments, Z¹ and Z² are different. In some embodiments, one of Z¹ and Z² is deuterium while the other is hydrogen.

In some embodiments, at least one of Z¹, Z², X¹, X², Y¹, Y², R³, R⁶, and R⁷ comprises deuterium.

In some embodiments, R⁶ and R⁷ are independently hydrogen, —CH₃, or -OCD₃.

In preferred embodiments, the psychedelic drug is at least one phenethylamine derivative selected from the group consisting of:

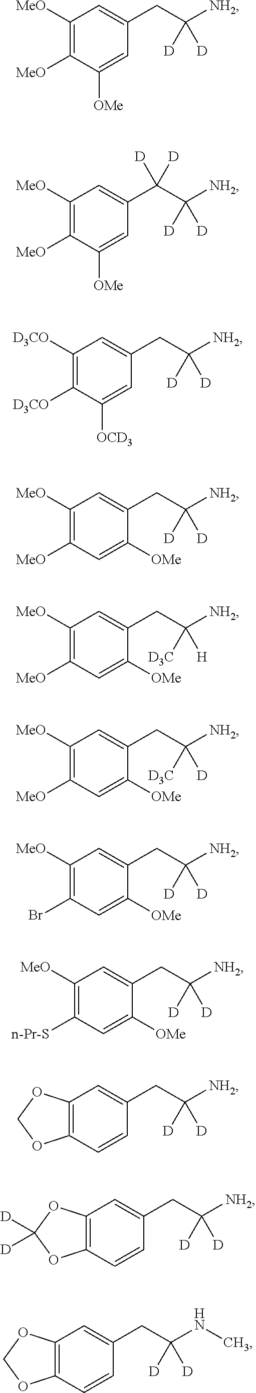

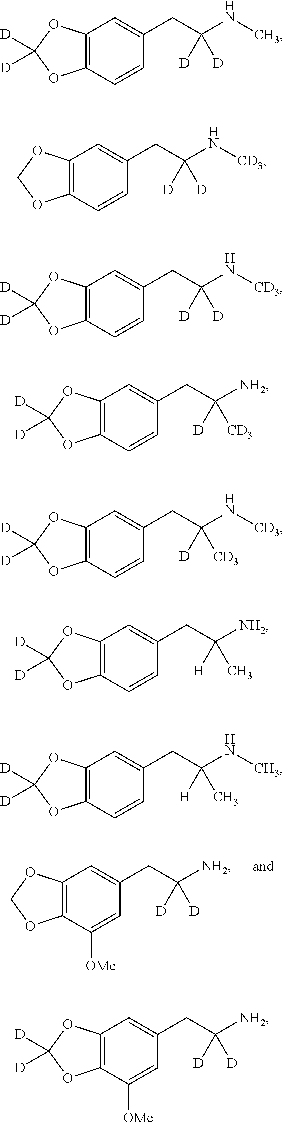

or a pharmaceutically acceptable salt, stereoisomer, solvate, or prodrug thereof.

Psychedelic drugs can also include N-substituted phenethylamines (NSPs) and derivatives thereof.

In some embodiments, the psychedelic drug is a compound of Formula (IV) or a pharmaceutically acceptable salt, stereoisomer, solvate, or prodrug thereof

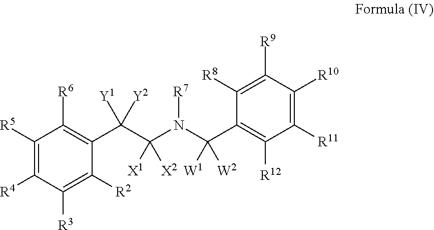

wherein:
R² and R³ are independently selected from the group consisting of hydrogen, deuterium, cyano, halogen, unsubstituted or substituted C₁-C₆ alkyl, —OR^{a}, and —SR^{a}, or R² and R³ together with the atoms to which they are attached optionally form an unsubstituted or substituted cycloalkyl, aryl, heterocycloalkyl, or heteroaryl;
R⁴ is selected from the group consisting of hydrogen, deuterium, cyano, halogen, unsubstituted or substituted C₁-C₆ alkyl, —OR^{a}, and —SR^{a};
R⁵ and R⁶ are independently selected from the group consisting of hydrogen, deuterium, cyano, halogen, unsubstituted or substituted C₁-C₆ alkyl, —OR^{a}, and —SR^{a}, or R⁵ and R⁶ together with the atoms to which they are attached optionally form an unsubstituted or substituted cycloalkyl, aryl, heterocycloalkyl, or heteroaryl;
W¹ and W² are independently selected from the group consisting of hydrogen, deuterium, and unsubstituted or substituted C₁-C₆ alkyl;
X¹ and X² are independently selected from the group consisting of hydrogen, deuterium, and unsubstituted or substituted C₁-C₆ alkyl; or X² and W¹ together with the atoms to which they are attached optionally form an unsubstituted or substituted heterocycloalkyl;
Y¹ and Y² are independently selected from the group consisting of hydrogen, deuterium, and unsubstituted or substituted C₁-C₆ alkyl;
R⁷ is selected from the group consisting of hydrogen, deuterium, and unsubstituted or substituted C₁-C₆ alkyl;
R⁸, R⁹, and R¹⁰ are independently selected from the group consisting of hydrogen, deuterium, hydroxyl, cyano, halogen, unsubstituted or substituted C₁-C₆ alkyl, —OR^{a}, and —SR^{a};
R¹¹ and R¹² are independently selected from the group consisting of hydrogen, deuterium, hydroxyl, cyano, halogen, unsubstituted or substituted C₁-C₆ alkyl, —OR^{a}, and —SR^{a}, or R¹¹ and R¹² together with the atoms to which they are attached optionally form an unsubstituted or substituted cycloalkyl, aryl, heterocycloalkyl, or heteroaryl; and
each R^{a} is independently selected from the group consisting of hydrogen, deuterium, and unsubstituted or substituted C₁-C₆ alkyl.

In some embodiments, R² is deuterium. In some embodiments, R² is hydrogen. In some embodiments, R² is halogen, for example —Br, —F, —Cl, or —I. In some embodiments, R² is cyano. In some embodiments, R² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R² is a substituted C₁-C₆ alkyl. When R² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R² is —OR^{a}. In some embodiments, R² is —SR^{a}.

In some embodiments, R³ is deuterium. In some embodiments, R³ is hydrogen. In some embodiments, R³ is halogen, for example —Br, —F, —Cl, or —I. In some embodiments, R³ is cyano. In some embodiments, R³ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R³ is a substituted C₁-C₆ alkyl. When R³ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R³ is —OR^{a}. In some embodiments, R³ is —SR^{a}.

In some embodiments, R² and R³ together with the atoms to which they are attached form an unsubstituted or substituted cycloalkyl, aryl, heterocycloalkyl, or heteroaryl.

In some embodiments, R⁴ is deuterium. In some embodiments, R⁴ is hydrogen. In some embodiments, R⁴ is halogen, for example —Br, —F, —Cl, or —I. In some embodiments, R⁴ is cyano. In some embodiments, R⁴ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R⁴ is —OR^{a}. In some embodiments, R⁴ is —SR^{a}. In some embodiments, R⁴ is —SMe, —SCD₃, —SCF₃, —SEt, —Sn—Pr, —SCH₂CH₂CF₃, —SCH₂CH₂CF₂H, —SCH₂CH₂CFH₂, -Me, —CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR^{a}, and R^{a} is C₁-C₆ alkyl, which is unsubstituted or substituted with one or more deuteriums.

In some embodiments, R⁵ is deuterium. In some embodiments, R⁵ is hydrogen. In some embodiments, R⁵ is halogen, for example —Br, —F, —Cl, or —I. In some embodiments, R⁵ is cyano. In some embodiments, R⁵ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁵ is a substituted C₁-C₆ alkyl. When R⁵ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁵ is —OR^{a}. In some embodiments, R⁵ is —SR^{a}. In some embodiments, R⁵ is hydrogen, —OMe, or —OCD₃. In some embodiments, R⁵ is hydrogen. In some embodiments, R⁵ is —OMe. In some embodiments, R⁵ is —OCD₃. In some embodiments, R⁵ is hydrogen, deuterium, halogen, —OR^{a}, or —SR^{a}, and R^{a} is C₁-C₆ alkyl, which is unsubstituted or substituted with one or more deuteriums.

In some embodiments, R⁶ is deuterium. In some embodiments, R⁶ is hydrogen. In some embodiments, R⁶ is halogen, for example —Br, —F, —Cl, or —I. In some embodiments, R⁶ is cyano. In some embodiments, R⁶ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁶ is a substituted C₁-C₆ alkyl. When R⁶ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁶ is —OR^{a}. In some embodiments, R⁶ is —SR^{a}. In some embodiments, R⁶ is hydrogen, —OMe, or —OCD₃. In some embodiments, R⁶ is hydrogen. In some embodiments, R⁶ is —OMe. In some embodiments, R⁶ is —OCD₃. In some embodiments, R⁶ is hydrogen, deuterium, halogen, —OR^{a}, or —SR^{a}, and R^{a} is C₁-C₆ alkyl, which is unsubstituted or substituted with one or more deuteriums.

In some embodiments, R⁵ and R⁶ together with the atoms to which they are attached optionally form an unsubstituted or substituted cycloalkyl, aryl, heterocycloalkyl, or heteroaryl.

W¹ and W² may be the same, or different. In some embodiments, W¹ and W² are the same. In some embodiments, W¹ and W² are hydrogen. In some embodiments, W¹ and W² are deuterium. In some embodiments, W¹ and W² are different. In some embodiments, W¹ is hydrogen or deuterium, and W² is a substituted or unsubstituted C₁-C₆ alkyl. In some embodiments, W² is an unsubstituted C₁-C₆ alkyl, examples of which include, but are not limited to, methyl, ethyl, and n-propyl, preferably methyl. In some embodiments, W² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of W¹ and W² is deuterium while the other is hydrogen.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of X¹ and X² is deuterium while the other is hydrogen.

In some embodiments, X² and W¹ together with the atoms to which they are attached form an unsubstituted or substituted heterocycloalkyl, e.g., a piperidine or pyrrolidine, which may be substituted or unsubstituted.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, Y¹ and Y² are different. In some embodiments, Y¹ is hydrogen or deuterium, and Y² is a substituted or unsubstituted C₁-C₆ alkyl. In some embodiments, Y² is an unsubstituted C₁-C₆ alkyl, examples of which include, but are not limited to, methyl, ethyl, and n-propyl, preferably methyl. In some embodiments, Y² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of Y¹ and Y² is deuterium while the other is hydrogen.

In some embodiments R⁷ is hydrogen. In some embodiments R⁷ is deuterium. In some embodiments R⁷ is an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more substituents, such as one or more deuterium (e.g., —CDH₂, —CD₂H, —CD₃).

R⁸, R⁹, and R¹⁰ may be the same, or different. In some embodiments, R⁸, R⁹, and R¹⁰ are the same. In some embodiments, R⁸, R⁹, and R¹⁰ are each different. In some embodiments, two of R⁸, R⁹, and R¹⁰ are the same.

In some embodiments, R⁸ is deuterium. In some embodiments, R⁸ is hydrogen. In some embodiments, R⁸ is halogen, for example —Br, —F, —Cl, or —I. In some embodiments, R⁸ is hydroxyl. In some embodiments, R⁸ is cyano. In some embodiments, R⁸ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁸ is a substituted C₁-C₆ alkyl. When R⁸ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁸ is —OR^{a}. In some embodiments, R⁸ is —SR^{a}. In some embodiments, R⁸ is hydrogen, —OMe, or —OCD₃. In some embodiments, R⁸ is hydrogen. In some embodiments, R⁸ is —OMe. In some embodiments, R⁸ is —OCD₃. In some embodiments, R⁸ is hydrogen, deuterium, halogen, —OR^{a}, or —SR^{a}, and R^{a} is C₁-C₆ alkyl, which is unsubstituted or substituted with one or more deuteriums.

In some embodiments, R⁹ is deuterium. In some embodiments, R⁹ is hydrogen. In some embodiments, R⁹ is halogen, for example —Br, —F, —Cl, or —I. In some embodiments, R⁹ is hydroxyl. In some embodiments, R⁹ is cyano. In some embodiments, R⁹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁹ is a substituted C₁-C₆ alkyl. When R⁹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁹ is —OR^{a}. In some embodiments, R⁹ is —SR^{a}. In some embodiments, R⁹ is hydrogen, —OMe, or —OCD₃. In some embodiments, R⁹ is hydrogen. In some embodiments, R⁹ is —OMe. In some embodiments, R⁹ is —OCD₃. In some embodiments, R⁹ is hydrogen, deuterium, halogen, —OR^{a}, or —SR^{a}, and R^{a} is C₁-C₆ alkyl, which is unsubstituted or substituted with one or more deuteriums.

In some embodiments, R¹⁰ is deuterium. In some embodiments, R¹⁰ is hydrogen. In some embodiments, R¹⁰ is halogen, for example —Br, —F, —Cl, or —I. In some embodiments, R¹⁰ is hydroxyl. In some embodiments, R¹⁰ is cyano. In some embodiments, R¹⁰ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹⁰ is a substituted C₁-C₆ alkyl. When R¹⁰ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be —CDH₂, —CD₂H, —CD₃, —CFH₂, —CF₂H, —CF₃, etc. In some embodiments, R¹⁰ is —OR^{a}. In some embodiments, R¹⁰ is —SR^{a}. In some embodiments, R¹⁰ is hydrogen, —OMe, or —OCD₃. In some embodiments, R¹⁰ is hydrogen. In some embodiments, R¹⁰ is —OMe. In some embodiments, R¹⁰ is —OCD₃. In some embodiments, R¹⁰ is hydrogen, deuterium, halogen, —OR^{a}, or —SR^{a}, and R^{a} is C₁-C₆ alkyl, which is unsubstituted or substituted with one or more deuteriums.

R¹¹ and R¹² may be the same or different. In some embodiments, R¹¹ is deuterium. In some embodiments, R¹¹ is hydrogen. In some embodiments, R¹¹ is halogen, for example —Br, —F, —Cl, or —I. In some embodiments, R¹¹ is hydroxyl. In some embodiments, R¹¹ is cyano. In some embodiments, R¹¹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹¹ is a substituted C₁-C₆ alkyl. When R¹¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹¹ is —OR^{a}. In some embodiments, R¹¹ is —SR^{a}. In some embodiments, R¹¹ is hydrogen, —OMe, or —OCD₃. In some embodiments, R¹¹ is hydrogen. In some embodiments, R¹¹ is —OMe. In some embodiments, R¹¹ is —OCD₃. In some embodiments, R¹¹ is hydrogen, deuterium, halogen, —OR^{a}, or —SR^{a}, and R^{a} is C₁-C₆ alkyl, which is unsubstituted or substituted with one or more deuteriums.

In some embodiments, R¹² is deuterium. In some embodiments, R¹² is hydrogen. In some embodiments, R¹² is halogen, for example —Br, —F, —Cl, or —I. In some embodiments, R¹² is hydroxyl. In some embodiments, R¹² is cyano. In some embodiments, R¹² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹² is a substituted C₁-C₆ alkyl. When R¹² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹² is —OR^{a}. In some embodiments, R¹² is —SR^{a}. In some embodiments, R¹² is hydrogen, —OMe, or —OCD₃. In some embodiments, R¹² is hydrogen. In some embodiments, R¹² is —OMe. In some embodiments, R¹² is —OCD₃. In some embodiments, R¹² is hydrogen, deuterium, halogen, —OR^{a}, or —SR^{a}, and R^{a} is C₁-C₆ alkyl, which is unsubstituted or substituted with one or more deuteriums.

In some embodiments, R¹¹ and R¹² together with the atoms to which they are attached form an unsubstituted or substituted cycloalkyl, aryl, heterocycloalkyl, or heteroaryl.

Each R^{a} may be, independently, hydrogen, deuterium, an unsubstituted C₁-C₆ alkyl (e.g., methyl, ethyl, n-propyl, isopropyl, n-butyl, isobutyl, sec-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, more than one R^{a} is present. In such cases, each R^{a} may be the same, or different. In some embodiments, each R^{a} is the same. In some embodiments, each R^{a} is different, e.g., one R^{a} is —CH₃, while another is —CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂CH₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of W¹, W², X¹, X², Y¹, Y², R², R³, R⁴, R⁵, R⁶, R⁷, R⁸, R⁹, R¹⁰, R¹¹, R¹² comprises deuterium.

In some embodiments, the psychedelic drug is a compound of Formula (IV-a) or a pharmaceutically acceptable salt, stereoisomer, solvate, or prodrug thereof

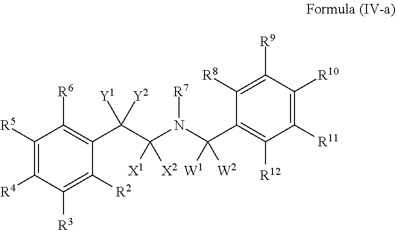

wherein:
X¹ and X² are deuterium; and
W¹, W², Y¹, Y², R², R³, R⁴, R⁵, R⁶, R⁷, R⁸, R⁹, R¹⁰, R¹¹, R¹², and R^{a} are as defined above for Formula (IV).

In some embodiments, at least one of W¹, W², Y¹, Y², R², R³, R⁴, R⁵, R⁶, R⁷, R⁸, R⁹, R¹⁰, R¹¹, and R¹² comprises deuterium.

In some embodiments, the psychedelic drug is a compound of Formula (IV-b) or a pharmaceutically acceptable salt, stereoisomer, solvate, or prodrug thereof

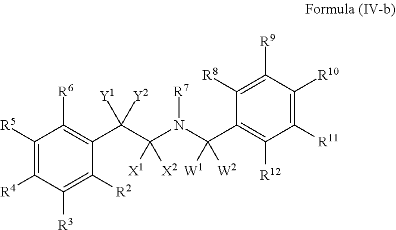

wherein:
W¹ and W² are deuterium; and
X¹, X², Y¹, Y², R², R³, R⁴, R⁵, R⁶, R⁷, R⁸, R⁹, R¹⁰, R¹¹, R¹², and R^{a} are as defined above for Formula (IV).

In some embodiments, at least one of X¹, X², Y¹, Y², R², R³, R⁴, R⁵, R⁶, R⁷, R⁸, R⁹, R¹⁰, R¹¹, R¹² comprises deuterium.

In some embodiments, the psychedelic drug is a compound of Formula (V) or a pharmaceutically acceptable salt, stereoisomer, solvate, or prodrug thereof

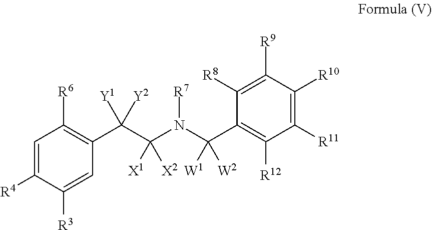

wherein:
R³ and R⁶ are —OR^{a};
R⁴ is selected from the group consisting of hydrogen, deuterium, cyano, halogen, unsubstituted or substituted C₁-C₆ alkyl, —OR^{a}, and —SR^{a}.
W¹ and W² are independently selected from the group consisting of hydrogen, deuterium, and unsubstituted or substituted C₁-C₆ alkyl;
X¹ and X² are independently selected from the group consisting of hydrogen, deuterium, and unsubstituted or substituted C₁-C₆ alkyl;
Y¹ and Y² are independently selected from the group consisting of hydrogen, deuterium, and unsubstituted or substituted C₁-C₆ alkyl;
R⁷ is selected from the group consisting of hydrogen, deuterium, and unsubstituted or substituted C₁-C₆ alkyl;
R⁸, R⁹, and R¹⁰ are independently selected from the group consisting of hydrogen, deuterium, hydroxyl, cyano, halogen, unsubstituted or substituted C₁-C₆ alkyl, —OR^{a}, and —SR^{a};
R¹¹ and R¹² are independently selected from the group consisting of hydrogen, deuterium, hydroxyl, cyano, halogen, unsubstituted or substituted C₁-C₆ alkyl, —OR^{a}, and —SR^{a}, or R¹¹ and R¹² together with the atoms to which they are attached optionally form an unsubstituted or substituted cycloalkyl, aryl, heterocycloalkyl, or heteroaryl; and
each R^{a} is independently selected from the group consisting of hydrogen, deuterium, and unsubstituted or substituted C₁-C₆ alkyl.

In some embodiments, R⁴ is deuterium. In some embodiments, R⁴ is hydrogen. In some embodiments, R⁴ is halogen, for example —Br, —F, —Cl, or —I. In some embodiments, R⁴ is cyano. In some embodiments, R⁴ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁴ is —OR^{a}. In some embodiments, R⁴ is —SR^{a}. In some embodiments, R⁴ is —SMe, —SCD₃, —SCF₃, —SEt, —Sn—Pr, —SCH₂CH₂CF₃, —SCH₂CH₂CF₂H, —SCH₂CH₂CFH₂, -Me, —CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR^{a}, and R^{a} is C₁-C₆ alkyl, which is unsubstituted or substituted with one or more deuteriums.

W¹ and W² may be the same, or different. In some embodiments, W¹ and W² are the same. In some embodiments, W¹ and W² are hydrogen. In some embodiments, W¹ and W² are deuterium. In some embodiments, W¹ and W² are different. In some embodiments, W¹ is hydrogen or deuterium, and W² is a substituted or unsubstituted C₁-C₆ alkyl. In some embodiments, W² is an unsubstituted C₁—C₆ alkyl, examples of which include, but are not limited to, methyl, ethyl, and n-propyl, preferably methyl. In some embodiments, W² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of W¹ and W² is deuterium while the other is hydrogen.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of X¹ and X² is deuterium while the other is hydrogen.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, Y¹ and Y² are different. In some embodiments, Y¹ is hydrogen or deuterium, and Y² is a substituted or unsubstituted C₁-C₆ alkyl. In some embodiments, Y² is an unsubstituted C₁-C₆ alkyl, examples of which include, but are not limited to, methyl, ethyl, and n-propyl, preferably methyl. In some embodiments, Y² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of Y¹ and Y² is deuterium while the other is hydrogen.

In some embodiments R⁷ is hydrogen. In some embodiments R⁷ is deuterium. In some embodiments R⁷ is an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more substituents, such as one or more deuterium (e.g., —CDH₂, —CD₂H, —CD₃).

R⁸, R⁹, and R¹⁰ may be the same, or different. In some embodiments, R⁸, R⁹, and R¹⁰ are the same. In some embodiments, R⁸, R⁹, and R¹⁰ are each different. In some embodiments, two of R⁸, R⁹, and R¹⁰ are the same.

In some embodiments, R⁸ is deuterium. In some embodiments, R⁸ is hydrogen. In some embodiments, R⁸ is halogen, for example —Br, —F, —Cl, or —I. In some embodiments, R⁸ is hydroxyl. In some embodiments, R⁸ is cyano. In some embodiments, R⁸ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁸ is a substituted C₁-C₆ alkyl. When R⁸ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁸ is —OR^{a}. In some embodiments, R⁸ is —SR^{a}. In some embodiments, R⁸ is hydrogen, —OMe, or —OCD₃. In some embodiments, R⁸ is hydrogen. In some embodiments, R⁸ is —OMe. In some embodiments, R⁸ is —OCD₃. In some embodiments, R⁸ is hydrogen, deuterium, halogen, —OR^{a}, or —SR^{a}, and R^{a} is C₁-C₆ alkyl, which is unsubstituted or substituted with one or more deuteriums.

In some embodiments, R⁹ is deuterium. In some embodiments, R⁹ is hydrogen. In some embodiments, R⁹ is halogen, for example —Br, —F, —Cl, or —I. In some embodiments, R⁹ is hydroxyl. In some embodiments, R⁹ is cyano. In some embodiments, R⁹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁹ is a substituted C₁-C₆ alkyl. When R⁹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁹ is —OR^{a}. In some embodiments, R⁹ is —SR^{a}. In some embodiments, R⁹ is hydrogen, —OMe, or —OCD₃. In some embodiments, R⁹ is hydrogen. In some embodiments, R⁹ is —OMe. In some embodiments, R⁹ is —OCD₃. In some embodiments, R⁹ is hydrogen, deuterium, halogen, —OR^{a}, or —SR^{a}, and R^{a} is C₁-C₆ alkyl, which is unsubstituted or substituted with one or more deuteriums.

In some embodiments, R¹⁰ is deuterium. In some embodiments, R¹⁰ is hydrogen. In some embodiments, R¹⁰ is halogen, for example —Br, —F, —Cl, or —I. In some embodiments, R¹⁰ is hydroxyl. In some embodiments, R¹⁰ is cyano. In some embodiments, R¹⁰ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹⁰ is a substituted C₁-C₆ alkyl. When R¹⁰ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹⁰ is —OR^{a}. In some embodiments, R¹⁰ is —SR^{a}. In some embodiments, R¹⁰ is hydrogen, —OMe, or —OCD₃. In some embodiments, R¹⁰ is hydrogen. In some embodiments, R¹⁰ is —OMe. In some embodiments, R¹⁰ is —OCD₃. In some embodiments, R¹⁰ is hydrogen, deuterium, halogen, —OR^{a}, or —SR^{a}, and R^{a} is C₁-C₆ alkyl, which is unsubstituted or substituted with one or more deuteriums.

R¹¹ and R¹² may be the same or different. In some embodiments, R¹¹ is deuterium. In some embodiments, R¹¹ is hydrogen. In some embodiments, R¹¹ is halogen, for example —Br, —F, —Cl, or —I. In some embodiments, R¹¹ is hydroxyl. In some embodiments, R¹¹ is cyano. In some embodiments, R¹¹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹¹ is a substituted C₁-C₆ alkyl. When R¹¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹¹ is —OR^{a}. In some embodiments, R¹¹ is —SR^{a}. In some embodiments, R¹¹ is hydrogen, —OMe, or —OCD₃. In some embodiments, R¹¹ is hydrogen. In some embodiments, R¹¹ is —OMe. In some embodiments, R¹¹ is —OCD₃. In some embodiments, R¹¹ is hydrogen, deuterium, halogen, —OR^{a}, or —SR^{a}, and R^{a} is C₁-C₆ alkyl, which is unsubstituted or substituted with one or more deuteriums.

In some embodiments, R¹² is deuterium. In some embodiments, R¹² is hydrogen. In some embodiments, R¹² is halogen, for example —Br, —F, —Cl, or —I. In some embodiments, R¹² is hydroxyl. In some embodiments, R¹² is cyano. In some embodiments, R¹² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹² is a substituted C₁-C₆ alkyl. When R¹² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹ is —OR^{a}. In some embodiments, R¹² is —SR^{a}. In some embodiments, R¹² is hydrogen, —OMe, or —OCD₃. In some embodiments, R¹² is hydrogen. In some embodiments, R¹² is —OMe. In some embodiments, R¹² is —OCD₃. In some embodiments, R¹² is hydrogen, deuterium, halogen, —OR^{a}, or —SR^{a}, and R^{a} is C₁-C₆ alkyl, which is unsubstituted or substituted with one or more deuteriums.

In some embodiments, R¹¹ and R¹² together with the atoms to which they are attached form an unsubstituted or substituted cycloalkyl, aryl, heterocycloalkyl, or heteroaryl.

Each R^{a} may be, independently, hydrogen, deuterium, an unsubstituted C₁-C₆ alkyl (e.g., methyl, ethyl, n-propyl, isopropyl, n-butyl, isobutyl, sec-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, more than one R^{a} is present. In such cases, each R^{a} may be the same, or different. In some embodiments, each R^{a} is the same. In some embodiments, each R^{a} is different, e.g., one R^{a} is —CH₃, while another is —CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂CH₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of W¹, W², X¹, X², Y¹, Y², R³, R⁴, R⁶, R⁷, R⁸, R⁹, R¹⁰, R¹¹, and R¹² comprises deuterium.

In some embodiments, the psychedelic drug is a compound of Formula (V-a) or a pharmaceutically acceptable salt, stereoisomer, solvate, or prodrug thereof

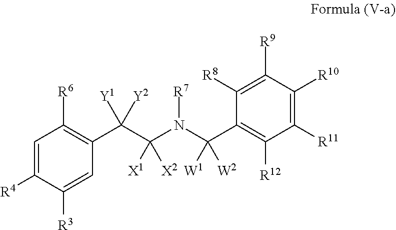

wherein:
R⁸, R⁹, R¹⁰, and R¹¹, are independently selected from the group consisting of hydrogen and deuterium;
R¹² is selected from the group consisting of hydrogen, deuterium, hydroxyl, cyano, halogen, unsubstituted or substituted C₁-C₆ alkyl, —OR^{a}, and —SR^{a}; and
W¹, W², X¹, X², Y¹, Y², R³, R⁴, R⁶, R⁷, and R^{a} are as defined above for Formula (V).

In some embodiments, at least one of W¹, W², X¹, X², Y¹, Y², R³, R⁴, R⁶, R⁷, R⁸, R⁹, R¹⁰, R¹¹, and R¹² comprises deuterium.

In some embodiments, the psychedelic drug is a compound of Formula (V-b) or a pharmaceutically acceptable salt, stereoisomer, solvate, or prodrug thereof

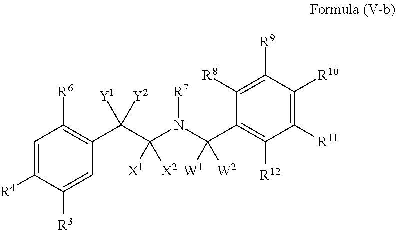

wherein:
R⁸, R⁹, and R¹⁰ are independently selected from the group consisting of hydrogen and deuterium;
R¹¹ and R¹² together with the atoms to which they are attached form an unsubstituted or substituted cycloalkyl, aryl, heterocycloalkyl, or heteroaryl; and
W¹, W², X¹, X², Y¹, Y², R³, R⁴, R⁶, R⁷, and R^{a} are as defined above for Formula (V).

In some embodiments, at least one of W¹, W², X¹, X², Y¹, Y², R³, R⁴, R⁶, R⁷, R⁸, R⁹, R¹⁰, R¹¹, and R¹² comprises deuterium.

In some embodiments, the psychedelic drug is a compound of Formula (VI) or a pharmaceutically acceptable salt, stereoisomer, solvate, or prodrug thereof

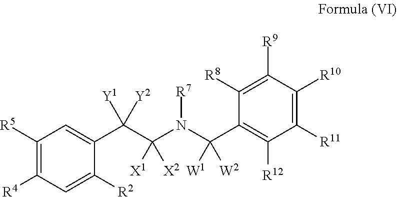

wherein:
R² and R⁵ are —OR^{a};
R⁴ is selected from the group consisting of hydrogen, deuterium, cyano, halogen, unsubstituted or substituted C₁-C₆ alkyl, —OR^{a}, and —SR^{a};
W¹ and W² are independently selected from the group consisting of hydrogen, deuterium, and unsubstituted or substituted C₁-C₆ alkyl;
X¹ and X² are independently selected from the group consisting of hydrogen, deuterium, and unsubstituted or substituted C₁-C₆ alkyl;
Y¹ and Y² are independently selected from the group consisting of hydrogen, deuterium, and unsubstituted or substituted C₁-C₆ alkyl;
R⁷ is selected from the group consisting of hydrogen, deuterium, and unsubstituted or substituted C₁-C₆ alkyl;
R⁸, R⁹, and R¹⁰ are independently selected from the group consisting of hydrogen, deuterium, hydroxyl, cyano, halogen, unsubstituted or substituted C₁-C₆ alkyl, —OR^{a}, and —SR^{a};
R¹¹ and R¹² are independently selected from the group consisting of hydrogen, deuterium, hydroxyl, cyano, halogen, unsubstituted or substituted C₁-C₆ alkyl, —OR^{a}, and —SR^{a}, or R¹¹ and R¹² together with the atoms to which they are attached optionally form an unsubstituted or substituted cycloalkyl, aryl, heterocycloalkyl, or heteroaryl; and
each R^{a} is independently selected from the group consisting of hydrogen, deuterium, and unsubstituted or substituted C₁-C₆ alkyl.

In some embodiments, R⁴ is deuterium. In some embodiments, R⁴ is hydrogen. In some embodiments, R⁴ is halogen, for example —Br, —F, —Cl, or —I. In some embodiments, R⁴ is cyano. In some embodiments, R⁴ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁴ is a substituted C₁-C₆ alkyl. When R⁴ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁴ is —OR^{a}. In some embodiments, R⁴ is —SR^{a}. In some embodiments, R⁴ is —SMe, —SCD₃, —SCF₃,-SEt, —Sn—Pr, —SCH₂CH₂CF₃, —SCH₂CH₂CF₂H, —SCH₂CH₂CFH₂,-Me, —CD₃, —CF₃, —OMe, —OCD₃, —OCF₃, —OCH₂CH₂CF₃, —OCH₂CH₂CF₂H, —OCH₂CH₂CFH₂, or —Br. In some embodiments, R⁴ is hydrogen, deuterium, halogen, —OR^{a}, or —SR^{a}, and R^{a} is C₁-C₆ alkyl, which is unsubstituted or substituted with one or more deuteriums.

W¹ and W² may be the same, or different. In some embodiments, W¹ and W² are the same. In some embodiments, W¹ and W² are hydrogen. In some embodiments, W¹ and W² are deuterium. In some embodiments, W¹ and W² are different. In some embodiments, W¹ is hydrogen or deuterium, and W² is a substituted or unsubstituted C₁-C₆ alkyl. In some embodiments, W² is an unsubstituted C₁-C₆ alkyl, examples of which include, but are not limited to, methyl, ethyl, and n-propyl, preferably methyl. In some embodiments, W² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of W¹ and W² is deuterium while the other is hydrogen.

X¹ and X² may be the same, or different. In some embodiments, X¹ and X² are the same. In some embodiments, X¹ and X² are hydrogen. In some embodiments, X¹ and X² are deuterium. In some embodiments, X¹ and X² are different. In some embodiments, X¹ is hydrogen or deuterium, and X² is a substituted or unsubstituted C₁-C₆ alkyl. In some embodiments, X² is an unsubstituted C₁-C₆ alkyl, examples of which include, but are not limited to, methyl, ethyl, and n-propyl, preferably methyl. In some embodiments, X² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of X¹ and X² is deuterium while the other is hydrogen.

Y¹ and Y² may be the same, or different. In some embodiments, Y¹ and Y² are the same. In some embodiments, Y¹ and Y² are hydrogen. In some embodiments, Y¹ and Y² are deuterium. In some embodiments, Y¹ and Y² are different. In some embodiments, Y¹ is hydrogen or deuterium, and Y² is a substituted or unsubstituted C₁-C₆ alkyl. In some embodiments, Y² is an unsubstituted C₁-C₆ alkyl, examples of which include, but are not limited to, methyl, ethyl, and n-propyl, preferably methyl. In some embodiments, Y² is a substituted C₁-C₆ alkyl. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, one of Y¹ and Y² is deuterium while the other is hydrogen.

In some embodiments R⁷ is hydrogen. In some embodiments R⁷ is deuterium. In some embodiments R⁷ is an unsubstituted C₁-C₆ alkyl (e.g., methyl, ethyl, n-propyl, isopropyl, n-butyl, isobutyl, sec-butyl, and hexyl) or a C₁-C₆ alkyl substituted with one or more substituents, such as one or more deuterium (e.g., —CDH₂, —CD₂H, —CD₃).

R⁸, R⁹, and R¹⁰ may be the same, or different. In some embodiments, R⁸, R⁹, and R¹⁰ are the same. In some embodiments, R⁸, R⁹, and R¹⁰ are each different. In some embodiments, two of R⁸, R⁹, and R¹⁰ are the same.

In some embodiments, R⁸ is deuterium. In some embodiments, R⁸ is hydrogen. In some embodiments, R⁸ is halogen, for example —Br, —F, —Cl, or —I. In some embodiments, R⁸ is hydroxyl. In some embodiments, R⁸ is cyano. In some embodiments, R⁸ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁸ is a substituted C₁-C₆ alkyl. When R⁸ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁸ is —OR^{a}. In some embodiments, R⁸ is —SR^{a}. In some embodiments, R⁸ is hydrogen, —OMe, or —OCD₃. In some embodiments, R⁸ is hydrogen. In some embodiments, R⁸ is —OMe. In some embodiments, R⁸ is —OCD₃. In some embodiments, R⁸ is hydrogen, deuterium, halogen, —OR^{a}, or —SR^{a}, and R^{a} is C₁-C₆ alkyl, which is unsubstituted or substituted with one or more deuteriums.

In some embodiments, R⁹ is deuterium. In some embodiments, R⁹ is hydrogen. In some embodiments, R⁹ is halogen, for example —Br, —F, —Cl, or —I. In some embodiments, R⁹ is hydroxyl. In some embodiments, R⁹ is cyano. In some embodiments, R⁹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R⁹ is a substituted C₁-C₆ alkyl. When R⁹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R⁹ is —OR^{a}. In some embodiments, R⁹ is —SR^{a}. In some embodiments, R⁹ is hydrogen, —OMe, or —OCD₃. In some embodiments, R⁹ is hydrogen. In some embodiments, R⁹ is —OMe. In some embodiments, R⁹ is —OCD₃. In some embodiments, R⁹ is hydrogen, deuterium, halogen, —OR^{a}, or —SR^{a}, and R^{a} is C₁-C₆ alkyl, which is unsubstituted or substituted with one or more deuteriums.

In some embodiments, R¹⁰ is deuterium. In some embodiments, R¹⁰ is hydrogen. In some embodiments, R¹⁰ is halogen, for example —Br, —F, —Cl, or —I. In some embodiments, R¹⁰ is hydroxyl. In some embodiments, R¹⁰ is cyano. In some embodiments, R¹⁰ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹⁰ is a substituted C₁-C₆ alkyl. When R¹⁰ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹⁰ is —OR^{a}. In some embodiments, R¹⁰ is —SR^{a}. In some embodiments, R¹⁰ is hydrogen, —OMe, or —OCD₃. In some embodiments, R¹⁰ is hydrogen. In some embodiments, R¹⁰ is —OMe. In some embodiments, R¹⁰ is —OCD₃. In some embodiments, R¹⁰ is hydrogen, deuterium, halogen, —OR^{a}, or —SR^{a}, and R^{a} is C₁-C₆ alkyl, which is unsubstituted or substituted with one or more deuteriums.

R¹¹ and R¹² may be the same or different. In some embodiments, R¹¹ is deuterium. In some embodiments, R¹¹ is hydrogen. In some embodiments, R¹¹ is halogen, for example —Br, —F, —Cl, or —I. In some embodiments, R¹¹ is hydroxyl. In some embodiments, R¹¹ is cyano. In some embodiments, R¹¹ is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹¹ is a substituted C₁-C₆ alkyl. When R¹¹ is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹¹ is —OR^{a}. In some embodiments, R¹¹ is —SR^{a}. In some embodiments, R¹¹ is hydrogen, —OMe, or —OCD₃. In some embodiments, R¹¹ is hydrogen. In some embodiments, R¹¹ is —OMe. In some embodiments, R¹¹ is —OCD₃. In some embodiments, R¹¹ is hydrogen, deuterium, halogen, —OR^{a}, or —SR^{a}, and R^{a} is C₁-C₆alkyl, which is unsubstituted or substituted with one or more deuteriums.

In some embodiments, R¹² is deuterium. In some embodiments, R¹² is hydrogen. In some embodiments, R¹² is halogen, for example —Br, —F, —Cl, or —I. In some embodiments, R¹² is hydroxyl. In some embodiments, R¹² is cyano. In some embodiments, R¹² is a an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R¹² is a substituted C₁-C₆ alkyl. When R¹² is a substituted C₁-C₆ alkyl, preferred substituents may include, but are not limited to, deuterium, halogen (e.g., fluorine), polar substituents such as hydroxyl or polyether substituents, etc. The alkyl group may contain one, or more than one, substituent. For example, when the alkyl group is a C₁ alkyl group (i.e., methyl group), the substituted C₁ alkyl group may be-CDH₂, —CD₂H, —CD₃, —CFH₂, —CF₂H, —CF₃, etc. In some embodiments, R¹² is —OR^{a}. In some embodiments, R¹² is —SR^{a}. In some embodiments, R¹² is hydrogen, —OMe, or —OCD₃. In some embodiments, R¹² is hydrogen. In some embodiments, R¹² is —OMe. In some embodiments, R¹² is —OCD₃. In some embodiments, R¹² is hydrogen, deuterium, halogen, —OR^{a}, or —SR^{a}, and R^{a} is C₁-C₆ alkyl, which is unsubstituted or substituted with one or more deuteriums.

In some embodiments, R¹¹ and R¹² together with the atoms to which they are attached form an unsubstituted or substituted cycloalkyl, aryl, heterocycloalkyl, or heteroaryl.

Each R^{a} may be, independently, hydrogen, deuterium, an unsubstituted C₁-C₆ alkyl (e.g., methyl, ethyl, n-propyl, isopropyl, n-butyl, isobutyl, sec-butyl, t-butyl, n-pentyl, neopentyl, and hexyl), or a substituted C₁-C₆ alkyl, with preferred substituents including, but not limited to, deuterium, halogen (e.g., fluorine), polar substituents such as hydroxyl or polyether substituents, etc. In some embodiments, R^{a} is a substituted or unsubstituted C₁-C₆ alkyl, preferably a C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, more than one R^{a} is present. In such cases, each R^{a} may be the same, or different. In some embodiments, each R^{a} is the same. In some embodiments, each R^{a} is different, e.g., one R^{a} is —CH₃, while another is —CD₃. In line with the above, examples of —OR^{a} or —SR^{a} may include, but are not limited to, —SMe, —SCD₃, —SCF₃, —SEt, —Sn—Pr, —SCH₂CH₂CF₃, —SCH₂CH₂CF₂H, —SCH₂CH₂CFH₂, —OMe, —OCD₃, —OCF₃, —OCH₂CH₂CF₃, —OCH₂CH₂CF₂H, and —OCH₂CH₂CFH₂.

In some embodiments, at least one of W¹, W², X¹, X², Y¹, Y², R², R⁴, R⁵, R⁷, R⁸, R⁹, R¹⁰, R¹¹, and R¹² comprises deuterium.

In some embodiments, the psychedelic drug is a compound of Formula (VI-a) or a pharmaceutically acceptable salt, stereoisomer, solvate, or prodrug thereof.

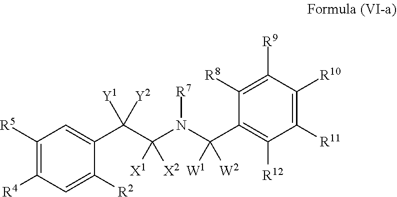

wherein:
R⁸, R⁹, R¹⁰, and R¹¹ are independently selected from the group consisting of hydrogen and deuterium;
R¹² is selected from the group consisting of hydrogen, deuterium, hydroxyl, cyano, halogen, unsubstituted or substituted C₁-C₆ alkyl, —OR^{a}, and —SR^{a}; and
W¹, W², X¹, X², Y¹, Y², R², R⁴, R⁵, R⁷, and R^{a} are as defined above for Formula (VI).

In some embodiments, at least one of W¹, W², X¹, X², Y¹, Y², R², R⁴, R⁵, R⁷, R⁸, R⁹, R¹⁰, R¹¹, and R¹² comprises deuterium.

In some embodiments, the psychedelic drug is a compound of Formula (VI-b) or a pharmaceutically acceptable salt, stereoisomer, solvate, or prodrug thereof

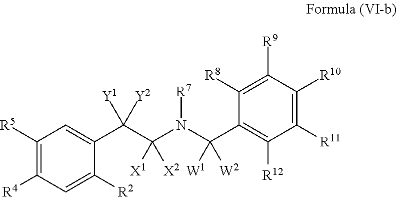

wherein:
R⁸, R⁹, and R¹⁰ are independently selected from the group consisting of hydrogen and deuterium;
R¹¹ and R¹² together with the atoms to which they are attached form an unsubstituted or substituted cycloalkyl, aryl, heterocycloalkyl, or heteroaryl; and
W¹, W², X¹, X², Y¹, Y², R², R⁴, R⁵, R⁷, and R^{a} are as defined above for Formula (VI).

In some embodiments, at least one of W¹, W², X¹, X², Y¹, Y², R², R⁴, R⁵, R⁷, R⁸, R⁹, R¹⁰, R¹¹, and R¹² comprises deuterium.

In some embodiments, the psychedelic drug is at least one phenethylamine derivative selected from the group consisting of

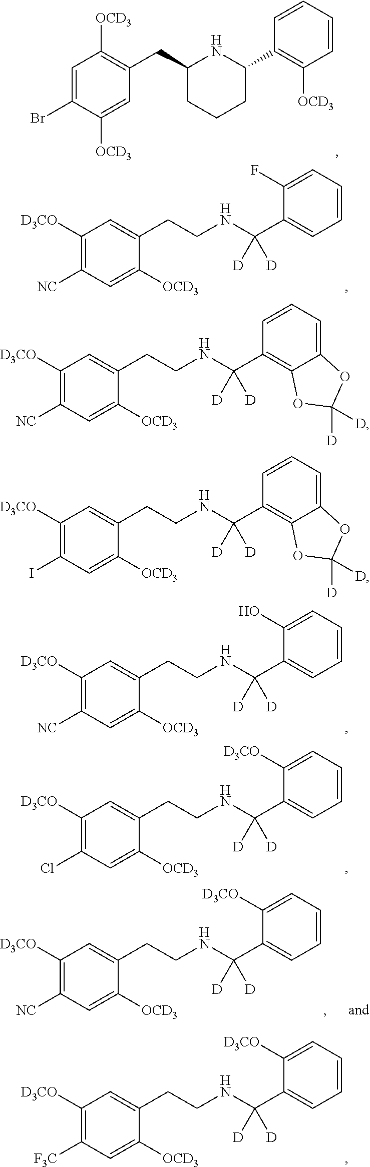

or a pharmaceutically acceptable salt, stereoisomer, solvate, or prodrug thereof.

In some embodiments, the psychedelic drug is a phenethylamine derivative including, but not limited to, MDMA, MDEA, MBDB, TMA, DOM, DOET, DOI, DOC; a tryptamine derivative, including, but not limited to, DMT, 5-MeO-DMT, psilocybin, psilocin; including a compound of Formula (I), Formula (II), Formula (II-a), Formula (II-b), Formula (II-c), Formula (II-d), Formula III, Formula (III-a), Formula (IV), Formula (IV-a), Formula (IV-b), Formula (V), Formula (V-a), Formula (V-b), Formula (VI), Formula (VI-a), Formula (VI-b), and any exemplary compounds described herein; as well as pharmaceutically acceptable salts, stereoisomers, and solvates thereof.

In some embodiments, the psychedelic drug is at least one selected from the group consisting of N,N-dimethyltryptamine (DMT), 5-hydroxy-N,N-dimethyltryptamine (5-OH-DMT), 5-methoxy-N,N-dimethyltryptamine (5-MeO-DMT), DMT-d₁₀(2-(1H-indol-3-yl)-N,N-bis(methyl-d₃)ethan-1-amine-1,1,2,2-d₄), 5-MeO-DMT-d₁₀ (2-(5-methoxy-1H-indol-3-yl)-N,N-bis(methyl-d₃)ethan-1-amine-1,1,2,2-d₄), and psilocin-d₁₀(3-(2-(bis(methyl-d₃)amino)ethyl-1,1,2,2-d₄)-1H-indol-4-ol), or a pharmaceutically acceptable salt or solvate thereof.

In some embodiments, the psychedelic drug is N,N-dimethyltryptamine (DMT), or a pharmaceutically acceptable salt or solvate thereof.

In some embodiments, the psychedelic drug is 5-hydroxy-N,N-dimethyltryptamine (5-OH-DMT), or a pharmaceutically acceptable salt or solvate thereof.

In some embodiments, the psychedelic drug is 5-methoxy-N,N-dimethyltryptamine (5-MeO-DMT), or a pharmaceutically acceptable salt or solvate thereof.

In some embodiments, the psychedelic drug comprises a deuterated tryptamine. In some embodiments, the psychedelic drug is 2-(1H-indol-3-yl)-N,N-bis(methyl-d₃)ethan-1-amine-1,1,2,2-d₄ (DMT-d₁₀), or a pharmaceutically acceptable salt or solvate thereof.

In some embodiments, the psychedelic drug is 2-(5-methoxy-1H-indol-3-yl)-N,N-bis(methyl-d₃)ethan-1-amine-1,1,2,2-d₄ (5-MeO-DMT-d₁₀), or a pharmaceutically acceptable salt or solvate thereof.

In some embodiments, the psychedelic drug is 2-(1H-indol-3-yl)-N,N-bis(methyl-d₃)ethan-1-amine-1,1-d₂ (DMT-d₈), or a pharmaceutically acceptable salt or solvate thereof.

In some embodiments, the psychedelic drug is 2-(5-(methoxy-d₃)-1H-indol-3-yl)-N,N-dimethylethan-1-amine-1,1-d₂ (5-MeO-DMT-d₅), or a pharmaceutically acceptable salt or solvate thereof.

In some embodiments, the psychedelic drug is 2-(5-(methoxy-d₃)-1H-indol-3-yl)-N,N-bis(methyl-d₃)ethan-1-amine-1,1,2,2-d₄ (5-MeO-DMT-d₁₃), or a pharmaceutically acceptable salt or solvate thereof.

Also disclosed herein is a pharmaceutically acceptable salt form of the compounds disclosed herein as the psychedelic drug. The acid used to form the pharmaceutically acceptable salt form may be a monoacid, a diacid, a triacid, a tetraacid, or may contain a higher number of acid groups. The acid groups may be, e.g., a carboxylic acid, a sulfonic acid, a phosphonic acid, or other acidic moieties containing at least one replaceable hydrogen atom. Examples of acids for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

In some embodiments, the salt is formed with N,N-dimethyltryptamine (DMT), 5-hydroxy-N,N-dimethyltryptamine (5-OH-DMT), 5-methoxy-N,N-dimethyltryptamine (5-MeO-DMT), 2-(1H-indol-3-yl)-N,N-bis(methyl-d₃)ethan-1-amine-1,1,2,2-d₄ (DMT-d₁₀), 2-(1H-indol-3-yl)-N,N-bis(methyl-d₃)ethan-1-amine-1,1-d₂ (DMT-d₈), 2-(5-methoxy-1H-indol-3-yl)-N,N-bis(methyl-d₃)ethan-1-amine-1,1,2,2-d₄ (5-MeO-DMT-d₁₀), 2-(5-(methoxy-d₃)-1H-indol-3-yl)-N,N-dimethylethan-1-amine-1,1-d₂ (5-MeO-DMT-d₅), or 2-(5-(methoxy-d₃)-1H-indol-3-yl)-N,N-bis(methyl-d₃)ethan-1-amine-1,1,2,2-d₄ (5-MeO-DMT-d₁₃).

In some embodiments, the psychedelic drug is a pharmaceutically acceptable salt of at least one compound selected from the group consisting of

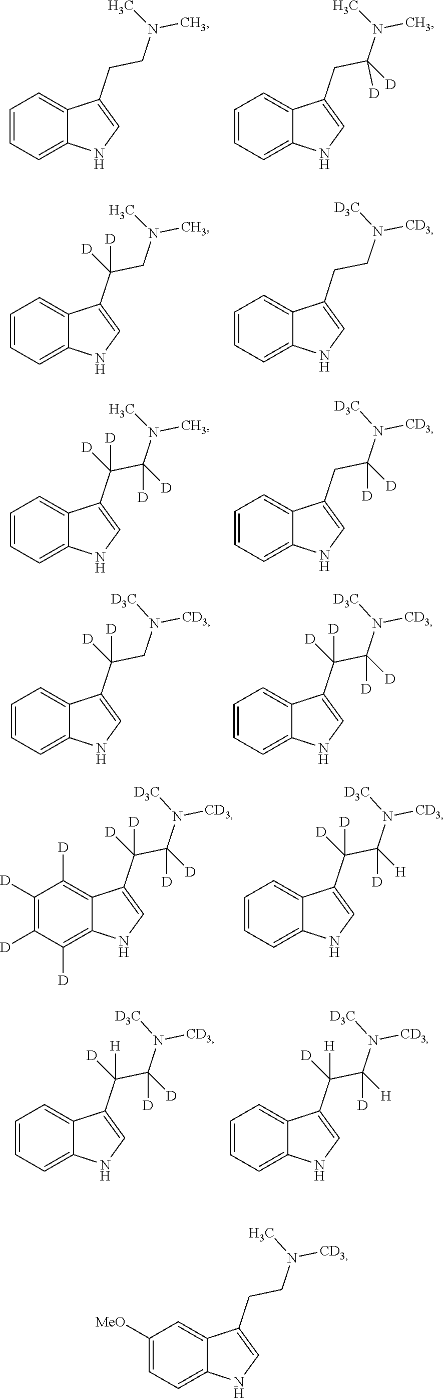

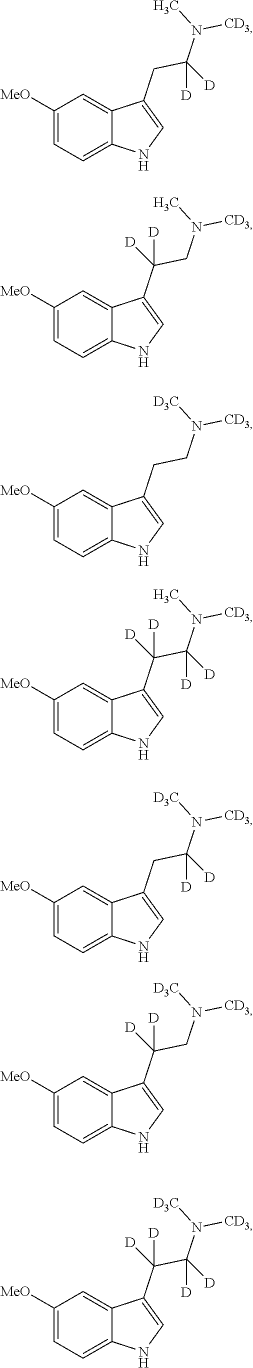

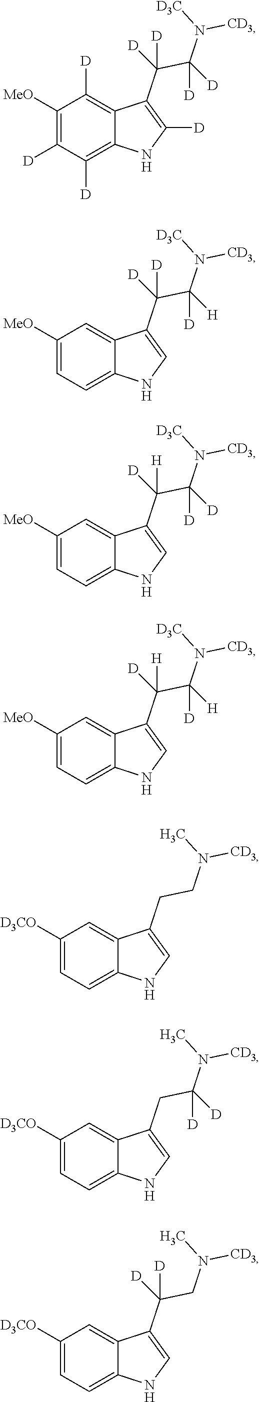

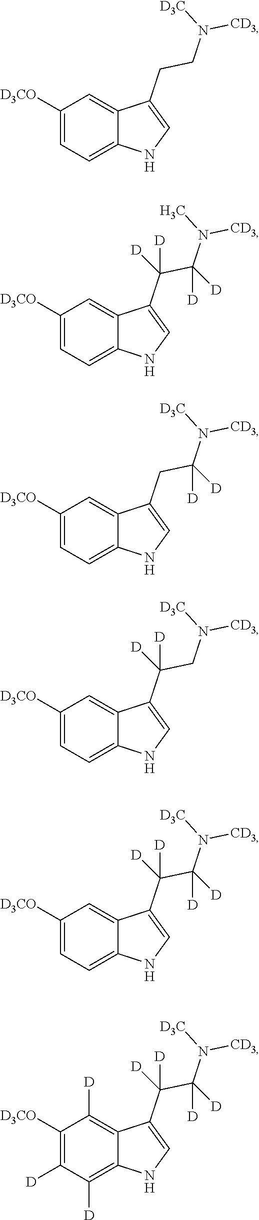

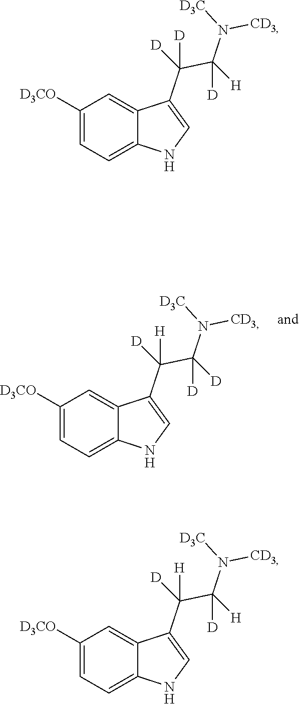

or a stereoisomer, solvate, or prodrug thereof.

In some embodiments, the pharmaceutically acceptable salt is a fumarate, a benzoate, a salicylate, a succinate, an oxalate, a glycolate, a hemi-oxalate, or a hemi-fumarate salt. In terms of providing desirable physical and pharmaceutical characteristics, such as those described above, preferred pharmaceutically acceptable salts are fumarate salts, benzoate salts, salicylates, and succinate salts of the compounds disclosed herein, e.g., the psychedelic drug, with fumarate, benzoate, and salicylate salts being particularly preferred.

In some embodiments, the pharmaceutically acceptable salt is a fumarate, a benzoate, a salicylate, a succinate, an oxalate, a glycolate, a hemi-oxalate, or a hemi-fumarate salt of N,N-dimethyltryptamine (DMT). In some embodiments, the pharmaceutically acceptable salt is a fumarate, a benzoate, a salicylate, a succinate, an oxalate, a glycolate, a hemi-oxalate, or a hemi-fumarate salt of 5-hydroxy-N,N-dimethyltryptamine (5-OH-DMT). In some embodiments, the pharmaceutically acceptable salt is a fumarate, a benzoate, a salicylate, a succinate, an oxalate, a glycolate, a hemi-oxalate, or a hemi-fumarate salt of 5-methoxy-N,N-dimethyltryptamine (5-MeO-DMT). In some embodiments, the pharmaceutically acceptable salt is a fumarate, a benzoate, a salicylate, a succinate, an oxalate, a glycolate, a hemi-oxalate, or a hemi-fumarate salt of 2-(1H-indol-3-yl)-N,N-bis(methyl-d₃)ethan-1-amine-1,1,2,2-d₄ (DMT-d₁₀). In some embodiments, the pharmaceutically acceptable salt is a fumarate, a benzoate, a salicylate, a succinate, an oxalate, a glycolate, a hemi-oxalate, or a hemi-fumarate salt of 2-(1H-indol-3-yl)-N,N-bis(methyl-d₃)ethan-1-amine-1,1-d₂ (DMT-d₈). In some embodiments, the pharmaceutically acceptable salt is a fumarate, a benzoate, a salicylate, a succinate, an oxalate, a glycolate, a hemi-oxalate, or a hemi-fumarate salt of 2-(5-methoxy-1H-indol-3-yl)-N,N-bis(methyl-d₃)ethan-1-amine-1,1,2,2-d₄ (5-MeO-DMT-d₁₀). In some embodiments, the pharmaceutically acceptable salt is a fumarate, a benzoate, a salicylate, a succinate, an oxalate, a glycolate, a hemi-oxalate, or a hemi-fumarate salt of 2-(5-(methoxy-d₃)-1H-indol-3-yl)-N,N-dimethylethan-1-amine-1,1-d₂ (5-MeO-DMT-d₅). In some embodiments, the pharmaceutically acceptable salt is a fumarate, a benzoate, a salicylate, a succinate, an oxalate, a glycolate, a hemi-oxalate, or a hemi-fumarate salt of 2-(5-(methoxy-d₃)-1H-indol-3-yl)-N,N-bis(methyl-d₃)ethan-1-amine-1,1,2,2-d₄ (5-MeO-DMT-d₁₃).

In some embodiments, the psychedelic drug is a pharmaceutically acceptable salt of DMT or a deuterated DMT and is a crystalline solid as disclosed in PCT/EP2023/050702, which is incorporated herein by reference in its entirety.

In some embodiments, the pharmaceutically acceptable salt is a fumarate salt of 2-(1H-indol-3-yl)-N,N-dimethylethan-1-amine (DMT, depicted below). In some embodiments, the fumarate salt of DMT is in a crystalline solid form characterized by an X-ray powder diffraction pattern containing at least three characteristic peaks at diffraction angles (2θ±0.2°) selected from 7.8°, 10.3°, 10.9°, 13.6°, 15.8°, 16.1°, 17.0°, 18.4°, 19.7°, 19.9°, 20.6°, 21.3°, 21.7°, 22.5°, 23.9°, 24.1°, 25.1°, 26.2°, 33.6°, and 34.9°, as determined by XRPD using a CuKα radiation source.

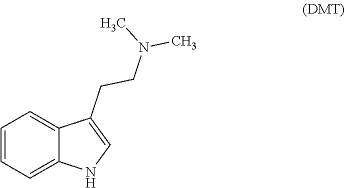

In some embodiments, the pharmaceutically acceptable salt is a benzoate salt of DMT. In some embodiments, the benzoate salt of DMT is in a crystalline solid form characterized by an X-ray powder diffraction pattern containing at least three characteristic peaks at diffraction angles (2θ±0.2°) selected from 9.6°, 11.1°, 12.6°, 13.5°, 15.8°, 16.1°, 17.1°, 17.9°, 19.8°, 20.1°, 20.8°, 21.2°, 22.7°, 23.8°, 24.6°, 26.9°, 29.2°, 32.3°, 35.1°, and 36.1°, as determined by XRPD using a CuKα radiation source.

In some embodiments, the pharmaceutically acceptable salt is a salicylate salt of DMT. In some embodiments, the salicylate salt of DMT is in a crystalline solid form characterized by an X-ray powder diffraction pattern containing at least three characteristic peaks at diffraction angles (2θ±0.2°) selected from 9.6°, 10.5°, 14.9°, 17.1°, 18.1°, 19.1°, 20.1°, 20.7°, 21.0°, 21.3°, 24.6°, 25.6°, 28.5°, 28.8°, 29.4°, 30.3°, 31.3°, 32.1°, 33.5°, and 34.4°, as determined by XRPD using a CuKα radiation source.

In some embodiments, the pharmaceutically acceptable salt is a succinate salt of DMT. In some embodiments, the succinate salt of DMT is in a crystalline solid form characterized by an X-ray powder diffraction pattern containing at least three characteristic peaks at diffraction angles (2θ±0.2°) selected from 9.8°, 11.7°, 14.3°, 14.7°, 17.0°, 17.4°, 19.6°, 20.6°, 22.3°, 22.6°, 22.9°, 23.1°, 23.4°, 24.9°, 25.2°, 26.3°, 26.8°, 27.3°, 27.7°, 28.8°, 29.1°, 30.9°, 31.5°, 33.8°, 34.5°, 36.5°, and 39.2°, as determined by XRPD using a CuKα radiation source.

In some embodiments, the pharmaceutically acceptable salt is an oxalate salt of DMT. In some embodiments, the oxalate salt of DMT is in a crystalline solid form characterized by an X-ray powder diffraction pattern containing at least three characteristic peaks at diffraction angles (2θ±0.2°) selected from 11.3°, 12.3°, 15.6°, 17.7°, 19.5°, 20.0°, 20.8°, 21.4°, 22.3°, 22.7°, 24.8°, 25.7°, 26.7°, 27.9°, 28.7°, 29.5°, 31.4°, 33.0°, 35.4°, 36.5°, and 38.6°, as determined by XRPD using a CuKα radiation source.

In some embodiments, the pharmaceutically acceptable salt is a glycolate salt of DMT. In some embodiments, the glycolate salt of DMT is in a crystalline solid form characterized by an X-ray powder diffraction pattern containing at least three characteristic peaks at diffraction angles (2θ±0.2°) selected from 8.2°, 12.2°, 12.9°, 15.8°, 16.3°, 17.8°, 19.2°, 20.1°, 21.7°, 23.6°, 24.4°, 24.6°, 24.9°, 26.0°, 26.6°, 27.8°, 29.6°, 30.2°, 32.0°, 32.3°, 33.0°, 33.9°, and 34.6°, as determined by XRPD using a CuKα radiation source.

In some embodiments, the pharmaceutically acceptable salt is a hemi-oxalate salt of DMT. In some embodiments, the hemi-oxalate salt of DMT is in a crystalline solid form characterized by an X-ray powder diffraction pattern containing at least three characteristic peaks at diffraction angles (2θ±0.2°) selected from 8.7°, 11.5°, 13.6°, 14.2°, 15.2°, 17.4°, 17.6°, 18.0°, 19.3°, 19.6°, 20.1°, 20.6°, 21.9°, 22.1°, 22.9°, 23.2°, 23.5°, 24.5°, 25.0°, 25.5°, 26.1°, 26.4°, 27.1°, 28.4°, 28.7°, 29.8°, 30.40, 30.7°, 31.4°, 31.8°, 33.4°, and 33.9°, as determined by XRPD using a CuKα radiation source.

In some embodiments, the pharmaceutically acceptable salt is a hemi-fumarate salt of DMT. In some embodiments, the hemi-fumarate salt of DMT is in a crystalline solid form characterized by an X-ray powder diffraction pattern containing at least three characteristic peaks at diffraction angles (2θ±0.2°) selected from 8.1°, 11.3°, 12.2°, 13.3°, 14.2°, 16.2°, 17.6°, 18.3°, 18.6°, 19.5°, 19.8°, 20.0°, 20.2°, 20.9°, 21.4°, 21.9°, 22.3°, 22.7°, 22.9°, 23.8°, 24.5°, 25.0°, 25.2°, 26.1°, 26.4°, 26.90, 28.4°, 28.8°, 29.5°, 29.8°, 30.9°, and 32.7°, as determined by XRPD using a CuKα radiation source.

In some embodiments, the pharmaceutically acceptable salt is a fumarate salt of 2-(1H-indol-3-yl)-N,N-bis(methyl-d₃)ethan-1-amine-1,1,2,2-d₄ (DMT-d₁₀, depicted below). In some embodiments, the fumarate salt of DMT-d₁₀ is in a crystalline solid form characterized by an X-ray powder diffraction pattern containing at least three characteristic peaks at diffraction angles (2θ±0.2°) selected from 7.8°, 10.3°, 10.9°, 12.5°, 13.6°, 14.6°, 15.2°, 15.5°, 15.8°, 16.1°, 16.6°, 17.0°, 18.40, 19.0°, 19.7°, 19.9°, 20.6°, 21.3°, 21.8°, 22.5°, 23.3°, 23.8°, 24.1°, 25.1°, 26.2°, 26.8°, 27.3°, 27.90, 28.3°, 28.9°, 29.3°, 29.6°, 29.9°, 30.6°, 31.0°, 31.3°, 32.4°, 32.9°, 33.3°, 33.6°, 34.3°, 34.9°, 35.70, 36.1°, 37.4°, 38.0°, and 38.5°, as determined by XRPD using a CuKα radiation source. In some embodiments, the fumarate salt of DMT-d₁₀ is in a crystalline solid form characterized by an X-ray powder diffraction pattern containing at least three characteristic peaks at diffraction angles (2θ±0.2°) selected from 7.8°, 10.3°, 10.9°, 13.6°, 15.8°, 16.1°, 17.0°, 18.4°, 19.7°, 19.9°, 20.6°, 21.3°, 21.8°, 22.5°, 23.8°, 24.1°, 25.1°, 26.2°, 33.6°, and 34.9°, as determined by XRPD using a CuKα radiation source. In some embodiments, the fumarate salt of DMT-d₁₀ is in a crystalline solid form characterized by an X-ray powder diffraction pattern containing at least three characteristic peaks at diffraction angles (2θ±0.2°) selected from 10.9°, 13.6°, 15.8°, 16.1°, 17.0°, 18.4°, 19.7°, 19.9°, 20.6°, 23.8°, 24.1°, and 25.1°, as determined by XRPD using a CuKα radiation source.

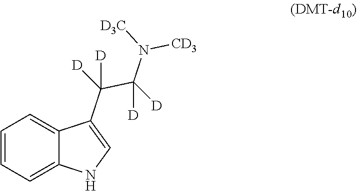 [SMILES: [2H]C([2H])([2H])N(C([2H])([2H])[2H])C([2H])([2H])C([2H])([2H])c1c[nH]c2ccccc12]

In some embodiments, the pharmaceutically acceptable salt is a benzoate salt of DMT-d₁₀. In some embodiments, the benzoate salt of DMT-d₁₀ is in a crystalline solid form characterized by an X-ray powder diffraction pattern containing at least three characteristic peaks at diffraction angles (2θ±0.2°) selected from 9.6°, 11.1°, 12.7°, 13.5°, 15.8°, 16.1°, 17.2°, 17.9°, 19.8°, 20.1°, 20.8°, 21.2°, 22.8°, 23.8°, 24.3°, 24.6°, 25.1°, 25.3°, 25.5°, 26.9°, 28.3°, 28.9°, 29.3°, 31.4°, 31.6°, 32.0°, 32.3°, 32.8°, 35.1°, and 36.1°, as determined by XRPD using a CuKα radiation source. In some embodiments, the benzoate salt of DMT-d₁₀ is in a crystalline solid form characterized by an X-ray powder diffraction pattern containing at least three characteristic peaks at diffraction angles (2θ±0.2°) selected from 9.6°, 11.1°, 12.7°, 13.5°, 15.8°, 16.1°, 17.2°, 17.9°, 19.8°, 20.1°, 20.8°, 21.2°, 22.8°, 23.8°, 24.6°, 26.9°, 29.3°, 32.3°, 35.1°, and 36.1°, as determined by XRPD using a CuKα radiation source. In some embodiments, the benzoate salt of DMT-d₁₀ is in a crystalline solid form characterized by an X-ray powder diffraction pattern containing at least three characteristic peaks at diffraction angles (2θ±0.2°) selected from 12.7°, 13.5°, 15.8°, 16.1°, 17.2°, 17.9°, 19.8°, 20.1°, 20.8°, 23.8°, 24.6°, 26.9°, 29.3°, and 35.1° as determined by XRPD using a CuKα radiation source.

In some embodiments, the pharmaceutically acceptable salt is a salicylate salt of DMT-d₁₀. In some embodiments, the salicylate salt of DMT-d₁₀ is in a crystalline solid form characterized by an X-ray powder diffraction pattern containing at least three characteristic peaks at diffraction angles (2θ±0.2°) selected from 9.6°, 10.5°, 11.4°, 12.3°, 13.4°, 14.2°, 14.9°, 15.6°, 16.1°, 17.1°, 18.1°, 18.7°, 19.1°, 20.1°, 20.8°, 21.1°, 21.3°, 22.2°, 22.6°, 23.7°, 24.6°, 25.2°, 25.6°, 26.1°, 26.4°, 27.4°, 27.5°, 27.8°, 28.5°, 28.8°, 29.4°, 29.7°, 30.3°, 31.0°, 31.3°, 32.1°, 32.7°, 33.1°, 33.5°, 34.4°, and 35.0°, as determined by XRPD using a CuKα radiation source. In some embodiments, the salicylate salt of DMT-d₁₀ is in a crystalline solid form characterized by an X-ray powder diffraction pattern containing at least three characteristic peaks at diffraction angles (2θ±0.2°) selected from 9.6°, 10.5°, 14.9°, 17.1°, 18.1°, 19.1°, 20.1°, 20.8°, 21.1°, 21.3°, 24.6°, 25.6°, 28.5°, 28.8°, 29.4°, 30.3°, 31.3°, 321°, 33.5°, and 34.4°, as determined by XRPD using a CuKα radiation source. In some embodiments, the salicylate salt of DMT-d₁₀ is in a crystalline solid form characterized by an X-ray powder diffraction pattern containing at least three characteristic peaks at diffraction angles (2θ±0.2°) selected from 9.6°, 14.9°, 17.1°, 18.1°, 19.1°, 20.1°, 20.8°, 21.3°, 24.6°, 25.6°, 28.5°, and 32.1°, as determined by XRPD using a CuKα radiation source.

Various methods and procedures for addition salt formation are known to those of ordinary skill in the art, any of which may be utilized in the present disclosure. In some embodiments, the method includes:
(a) suspending a free base of the psychedelic drug in a solvent or mixture of solvents;
(b) contacting an acid with the psychedelic drug to provide a mixture;
(c) optionally heating the mixture;
(d) optionally cooling the mixture; and
(e) isolating the salt.

Various solvents may be used in the disclosed methods, including one or more protic solvents, one or more aprotic solvents, or mixtures thereof. In some embodiments, the solvent(s) used in the method of preparing the salt is/are a protic solvent(s). In some embodiments, the solvent used in the method of preparing the salt is selected from the group consisting of methanol, ethanol, propanol, isopropanol, butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, and octane, and combinations thereof. In some embodiments, the solvent is ethanol.

Suitable acids for use in the preparation of pharmaceutically acceptable acid addition salts may include those described heretofore. The acid may be an inorganic acid or an organic acid, with organic acids being preferred. In some embodiments, the acid is an organic acid selected from the group consisting of fumaric acid, benzoic acid, salicylic acid, succinic acid, oxalic acid, and glycolic acid. In some embodiments, the acid is an organic acid selected from the group consisting of fumaric acid, benzoic acid, salicylic acid, and succinic acid, with fumaric acid, benzoic acid, and salicylic acid being preferred.

In some embodiments, a stoichiometric (or superstoichiometric) quantity of the acid is contacted with the free base of the psychedelic drug. In some embodiments, a sub-stoichiometric (e.g., 0.5 molar equivalents) quantity of the acid is contacted with the free base of the psychedelic drug. The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

In some embodiments, the psychedelic drug of the present disclosure, or any pharmaceutically acceptable salts, stereoisomers, or prodrugs thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. In one non-limiting example, as a hydrate, the psychedelic drug may be a monohydrate, a dihydrate, etc. Solvates of the compounds herein also include solution-phase forms. Thus, in some embodiments, the present disclosure provides solution-phase compositions of the psychedelic drug of the present disclosure, or any pharmaceutically acceptable salts, stereoisomers, or prodrugs thereof, which are in solvated form, preferably fully solvated form. For example, pharmaceutically acceptable salt forms of the psychedelic drug can be prepared in solution-phase, whereby the salt is pre-formed as a solid and then dissolved in solvent (e.g., water). Alternatively, pharmaceutically acceptable salt forms of the psychedelic drug can be prepared in solution-phase, by mixing the psychedelic drug (free base) with an appropriate acid in solvent (e.g., water) thereby forming the solvated salt form in-situ. If desired, these preparations can be stored as a solution, such as in the form of an aqueous solution, an organic solvent solution, or a mixed aqueous-organic solvent solution, for prolonged periods of time without appreciable degradation or physical changes, such as oiling out of solution. Solvents which can be used to form the solution-phase compositions can be any one or more solvents set forth herein, e.g., water, ethanol, etc. In some embodiments, the solution-phase composition is an aqueous solution-phase composition comprising the psychedelic drug, or a pharmaceutically acceptable salt, stereoisomer, or prodrug thereof, solvated with water.

The psychedelic drug may contain a stereogenic center. In such cases, the compounds may exist as different stereoisomeric forms, even though the chemical Formulae/name are drawn/written without reference to stereochemistry. Accordingly, the present disclosure includes all possible stereoisomers and includes not only racemic compounds but the individual enantiomers (enantiomerically pure compounds), individual diastereomers (diastereomerically pure compounds), and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

In some embodiments, the compounds described herein, e.g., the psychedelic drug, is non-stereogenic. In some embodiments, the compounds described herein, e.g., the psychedelic drug, is racemic. In some embodiments, the compounds described herein, e.g., the psychedelic drug, is enantiomerically enriched (one enantiomer is present in a higher percentage), including enantiomerically pure. In some embodiments, the compounds described herein, e.g., the psychedelic drug, is provided as a single diastereomer. In some embodiments, the compounds described herein, e.g., psychedelic drug, is provided as a mixture of diastereomers. When provided as a mixture of diastereomers, the mixtures may include equal mixtures, or mixtures which are enriched with a particular diastereomer (one diastereomer is present in a higher percentage than another).

In some embodiments, the psychedelic drug is chemically pure, for example has a chemical purity of greater than 90%, 92%, 94%, 96%, 97%, 98%, or 99% by UPLC or HPLC. In some embodiments, the psychedelic drug has no single impurity of greater than 1%, greater than 0.5%, greater than 0.4%, greater than 0.3%, or greater than 0.2%, measured by UPLC or HPLC. In some embodiments, the psychedelic drug has a chemical purity of greater than 97 area %, greater than 98 area %, or greater than 99 area % by UPLC or HPLC. In some embodiments, the psychedelic drug has no single impurity greater than 1 area %, greater than 0.5 area %, greater than 0.4 area %, greater than 0.3 area %, or greater than 0.2 area % as measured by UPLC or HPLC.

In some embodiments, a 5-HT_{2A} receptor agonist and an NMDA receptor antagonist are used in combination, in some cases combined in a single pharmaceutical composition. It has been surprisingly found that the 5-HT_{2A} receptor agonist and NMDA receptor antagonist (e.g., ketamine, nitrous oxide, memantine, amantadine, dextromethorphan (DXM), phencyclidine (PCP), methoxetamine (MXE), dizocilpine (MK-801), esmethadone, noble gases with NMDA receptor antagonist activity such as xenon (Xe) and/or argon (Ar)) provide enhanced therapeutic efficacy while improving patient experience. For example, the 5-HT_{2A} receptor agonist may be used in conjunction with the NMDA receptor antagonist (e.g., nitrous oxide, xenon and/or argon) to reduce or eliminate psychological disorders such as acute psychedelic crisis (a bad trip), and dysphoric physiological and psychological side effects or other adverse events that can accompany psychedelic psychotherapy sessions. In some embodiments, a 5-HT_{2A} receptor agonist is used in combination with nitrous oxide. In some embodiments, a noble gas (e.g., xenon and/or argon) is used together with nitrous oxide, or as a replacement for nitrous oxide. Thus, any embodiment described with nitrous oxide herein may be replaced with a noble gas such as xenon, argon, or both.

Dosage of psychedelic drugs can vary. A pharmaceutical composition can include compositions wherein the psychedelic drug is contained in a therapeutically effective amount. An “effective amount” or a “therapeutically effective amount” is a sufficient amount of the drug to treat or ameliorate a condition, disorder, or disease. The actual amount effective for a particular application can depend, inter alia, on the condition being treated, the psychedelic drug utilized, etc. The dosage and frequency (single or multiple doses) of psychedelic drug administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans can be determined (e.g., from animal models). For example, a dose for humans can be formulated to achieve a concentration that has been found to be effective in animals. The dosage in humans can be adjusted by monitoring response of the human to the treatment and adjusting the dosage upwards or downwards. Determination of the dosage and frequency of psychedelic drug administered is readily within the ability of one of ordinary skill in the medical field, taking into account the various factors noted above.

Dosages can be varied depending upon the requirements of the subject and the psychedelic drug being used. The dose administered to a subject, in the context of the psychedelic drugs presented herein, should be sufficient to affect a beneficial therapeutic response in the subject over time. The size of the dose also will be determined by the existence, nature, and extent of any adverse side effects. Treatment can be initiated with smaller dosages, which are less than the optimum dose of the psychedelic drug. Thereafter, the dosage can be increased by small increments until the optimum effect under the circumstances is reached.

Dosage amounts and intervals can be adjusted individually to provide levels of the administered compounds effective for the particular clinical indication being treated. This will provide a therapeutic regimen that is commensurate with the severity of the individual's disease state.

Administration may follow a continuous administration schedule, or an intermittent administration schedule. The administration schedule may be varied depending on the psychedelic drug(s) employed, the condition being treated, the administration route, etc. For example, administration of a psychedelic drug may be performed once a day (QD), or in divided dosages throughout the day, such as 2-times a day (BID), 3-times a day (TID), 4-times a day (QID), or more. In some embodiments administration may be performed nightly (QHS). In some embodiments, administration is performed as needed (PRN). Administration may also be performed on a weekly basis, e.g., once a week, twice a week, three times a week, four times a week, every other week, every two weeks, etc. The administration schedule may also designate a defined number of treatments per treatment course, for example, the psychedelic drug may be administered 1 time, 2 times, 3 times, 4 times, 5 times, 6 times, 7 times, or 8 times per treatment course. Other administration schedules may also be deemed appropriate using sound medical judgement.

The dosing can be continuous (7 days of administration in a week) or intermittent, for example, depending on the pharmacokinetics and a particular subject's clearance/accumulation of the psychedelic drug(s). If intermittently, the schedule may be, for example, 4 days of administration and 3 days off (rest days) in a week or any other intermittent dosing schedule deemed appropriate using sound medical judgement. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

An effective prophylactic or therapeutic treatment regimen can be planned that does not cause substantial toxicity and yet is effective to treat the clinical symptoms demonstrated by the particular patient. This planning can involve the choice of psychedelic drug by considering factors such as compound potency, relative bioavailability, patient body weight, presence and severity of adverse side effects, mode of administration, and the toxicity profile of the selected psychedelic drug.

A therapeutically or prophylactically effective fixed dose herein may vary depending on the variety of factors described above, but is typically that which provides e.g., via aerosol inhalation, the psychedelic drug in an amount of about 1 μg to about 1000 mg or more (or any range between about 1 μg to about 1000 mg), e.g., about 1 μg, about 2 μg, about 5 μg, about 6 μg, about 10 μg, about 13 μg, about 15 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 400 μg, about 500 μg, about 1.0 mg, about 2.0 mg, about 3.0 mg, about 4.0 mg, about 5.0 mg, about 6.0 mg, about 7.0 mg, about 8.0 mg, about 9.0 mg, about 10.0 mg, about 20.0 mg, about 30.0 mg, about 40.0 mg, about 50.0 mg, about 60.0 mg, about 70.0 mg, about 80.0 mg, about 90.0 mg, about 100.0 mg, about 200.0 mg, about 300.0 mg, about 400.0 mg, about 500.0 mg, about 600.0 mg, about 700.0 mg, about 800.0 mg, about 900.0 mg, about 1000.0 mg, or more, per inhalation session. In some embodiments, a subject can have 1, 2, 3, 4, 5 or more inhalation sessions a day. In some embodiments, a subject can have 1, 2, 3, 4, 5 or more inhalation sessions every other day, twice a week, or three times a week. In some embodiments, a subject can have 1, 2, 3, 4, 5 or more inhalation sessions every other month, twice a month, three times a month, or four times a month. In some embodiments, a subject can have 1, 2, 3, 4, 5, 6, 7, 8, or more inhalation sessions per treatment course, such as within a 28-day time period.

A therapeutically or prophylactically effective weight based dose herein may vary depending on the variety of factors described above, but is typically that which provides the psychedelic drug in an amount of about 0.00001 mg to about 10 mg per kilogram body weight of the recipient, or any range in between, e.g., about 0.00001 mg/kg, about 0.00005 mg/kg, about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.12 mg/kg, about 0.15 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg.

In some embodiments, the psychedelic drug may be administered at a psychedelic dose, for example, at a dose of from greater than about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, and up to about 5 mg/kg, about 4 mg/kg, about 3 mg/kg, about 2.5 mg/kg, about 2 mg/kg, about 1 mg/kg, about 0.95 mg/kg, about 0.9 mg/kg, about 0.85 mg/kg, about 0.8 mg/kg, about 0.75 mg/kg, about 0.7 mg/kg, about 0.65 mg/kg, about 0.6 mg/kg, about 0.55 mg/kg. The aforementioned psychedelic doses are typically administered 1 time, 2 times, 3 times, 4 times, 5 times, 6 times, 7 times, or 8 times in any one course of treatment. Courses can be repeated as necessary, with or without a drug holiday. Such treatment regimens may be accompanied by psychotherapy, before, during, and/or after the psychedelic dose. These treatments may be appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder).

In some embodiments, the psychedelic drug may be administered at a serotonergic, but sub-psychedelic dose, for example at a dose of from about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, and up to about 0.1 mg/kg, about 0.09 mg/kg, about 0.083 mg/kg, about 0.08 mg/kg, about 0.075 mg/kg, about 0.07 mg/kg, about 0.06 mg/kg, about 0.05 mg/kg, about 0.04 mg/kg, about 0.03 mg/kg, about 0.02 mg/kg of the active ingredient(s). Typically, sub-psychedelic doses are administered every day, for a treatment course (e.g., 1 month). However, there is no limitation on the number of doses at sub-psychedelic doses—dosing can be less frequent or more frequent as deemed appropriate. Here, doses would be adapted to provide sub-psychedelic blood levels of the psychedelic drug. Courses can be repeated as necessary, with or without a drug holiday.

Also disclosed herein is a pharmaceutical composition comprising the psychedelic drug and optionally another active ingredient (e.g., an NMDA receptor antagonist). Typically, the pharmaceutical composition is also formulated with other chemical components, such as pharmaceutically acceptable excipients. One purpose of a pharmaceutical composition is to facilitate administration of the active ingredient(s) disclosed herein in any of its embodiments to a subject in need thereof. In some embodiments, the psychedelic drug is only active ingredient present in the pharmaceutical composition.

The pharmaceutical composition may contain at least 0.0001 wt. %, at least 0.001 wt. %, at least 0.01 wt. %, at least 0.05 wt. %, at least 0.1 wt. %, at least 0.5 wt. %, at least 5 wt. %, at least 10 wt. %, at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, at least 60 wt. %, at least 65 wt. %, at least 70 wt. %, at least 75 wt. %, at least 80 wt. %, at least 85 wt. %, at least 90 wt. %, at least 95 wt. %, at least 99 wt. %, or at least 99.9 wt. % of the psychedelic drug, relative to a total weight of the pharmaceutical composition. In some embodiments, the pharmaceutical composition comprises up to 99 wt. %, up to 98 wt. %, up to 97 wt. %, up to 95 wt. %, up to 90 wt. %, up to 85 wt. %, up to 80 wt. %, up to 75 wt. %, up to 70 wt. %, up to 65 wt. %, up to 60 wt. %, up to 55 wt. %, up to 50 wt. %, up to 45 wt. %, up to 40 wt. %, up to 35 wt. %, up to 30 wt. %, up to 25 wt. %, up to 20 wt. %, up to 15 wt. %, up to 10 wt. %, up to 5 wt. % of the psychedelic drug, relative to a total weight of the pharmaceutical composition. Any range between any of these recited end points may be used.

A pharmaceutical composition comprising a psychedelic drug can be prepared and administered in a wide variety of dosage formulations. Liquid form preparations include solutions, suspensions, emulsions, micelles, liposomes, microspheres, nanosystems, and solid forms suitable for solutions or suspensions in liquid prior to inhalation. Solid form preparations suitable for inhalation administration, such as dry powders, are also disclosed herein. Such dosage forms can be prepared according to conventional methods known to those skilled in the art of pharmaceutical science.

In embodiments where the pharmaceutical composition is formulated with a deuterated psychedelic drug, such as a compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), or Formula (VI-b) comprising at least one deuterium atom, the pharmaceutical composition may comprise a single isotopologue or an isotopologue mixture of compounds, or pharmaceutically acceptable salts, solvates, or stereoisomers thereof. In some embodiments, a subject compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), or Formula (VI-b) may be present in the pharmaceutical composition at a purity of at least 50% by weight, at least 60% by weight, at least 70% by weight, at least 80% by weight, at least 90% by weight, at least 95% by weight, at least 99% by weight, based on a total weight of isotopologues of the compound of Formula (I), Formula (II), Formula (II-a), Formula (II-b), Formula (II-c), Formula (II-d), Formula (III), Formula (III-a), Formula (IV), Formula (IV-a), Formula (IV-b), Formula (V), Formula (V-a), Formula (V-b), Formula (VI), Formula (VI-a), or Formula (VI-b) present in the pharmaceutical composition. For example, a pharmaceutical composition formulated with DMT-d₁₀, as the subject compound, may additionally contain isotopologues of the subject compound, e.g., DMT-d₉, a DMT-d₈, etc., as free-base or salt forms, stereoisomers, solvates, or mixtures thereof. In some embodiments, the composition is substantially free of other isotopologues of the compound, in either free base or salt form, e.g., the composition has less than 20, 15, 10, 9, 8, 7, 6, 5, 4, 3, 2 or 1 or 0.5 mole percent of other isotopologues of the compound.

In some embodiments, any position indicated in the compound as having deuterium has a minimum deuterium incorporation that is greater than that found naturally occurring in hydrogen (natural abundance of about 0.016 atom % deuterium). In some embodiments, any position indicated in the compound as having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration.

In some embodiments, the pharmaceutical composition comprises at least two psychedelic drugs (referred to herein as a “psychedelic drug mixture”). Such pharmaceutical compositions may optionally further comprise one or more NMDA receptor antagonists when it is desirable to administer both types of active ingredients in the same dosage form.

In some embodiments, the pharmaceutical composition comprises a psychedelic drug mixture comprising: (i) DMT-d₁₀, i.e., 2-(1H-indol-3-yl)-N,N-bis(methyl-d₃)ethan-1-amine-1,1,2,2-d₄, or a pharmaceutically acceptable salt, solvate, or prodrug thereof; (ii) DMT-d₉, i.e., one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) DMT-d₈, i.e., one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof. In some embodiments, the psychedelic drug mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, or any range therebetween, based on a total weight of the psychedelic drug mixture, of (i) DMT-d₁₀, i.e., 2-(1H-indol-3-yl)-N,N-bis(methyl-d₃)ethan-1-amine-1,1,2,2-d₄, or a pharmaceutically acceptable salt, solvate, or prodrug thereof. In some embodiments, the psychedelic drug mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, or any range therebetween, based on a total weight of the psychedelic drug mixture, of (ii) DMT-d₉, i.e., one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof. In some embodiments, the psychedelic drug mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the psychedelic drug mixture, of (iii) DMT-d₈, i.e., one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof. In some embodiments, the psychedelic drug mixture consists of or consists essentially of (i) DMT-d₁₀, i.e., 2-(1H-indol-3-yl)-N,N-bis(methyl-d₃)ethan-1-amine-1,1,2,2-d₄, or a pharmaceutically acceptable salt, solvate, or prodrug thereof; and (ii) DMT-d₉, i.e., one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof.

In some embodiments, the pharmaceutical composition comprises a psychedelic drug mixture comprising: (i) 5-MeO-DMT-d₁₀, i.e., 2-(5-methoxy-1H-indol-3-yl)-N,N-bis(methyl-d₃)ethan-1-amine-1,1,2,2-d₄, or a pharmaceutically acceptable salt, solvate, or prodrug thereof; (ii) 5-MeO-DMT-d₉, i.e., one or more of 2-(5-methoxy-1H-indol-3-yl)-N,N-bis(methyl-d₃)ethan-1-amine-1,2,2-d₃ and 2-(5-methoxy-1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) 5-MeO-DMT-d₈, i.e., one or more of 2-(5-methoxy-1H-indol-3-yl)-N,N-bis(methyl-d₃)ethan-1-amine-1,1-d₂, 2-(5-methoxy-1H-indol-3-yl)-N,N-bis(methyl-d₃)ethan-1-amine-2,2-d₂, and 2-(5-methoxy-1H-indol-3-yl)-N,N-bis(methyl-d₃)ethan-1-amine-1,2-d₂, or a pharmaceutically acceptable salt, stereoisomer, solvate, or prodrug thereof. In some embodiments, the psychedelic drug mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, or any range therebetween, based on a total weight of the psychedelic drug mixture, of (i) 5-MeO-DMT-d₁₀, i.e., 2-(5-methoxy-1H-indol-3-yl)-N,N-bis(methyl-d₃)ethan-1-amine-1,1,2,2-d₄, or a pharmaceutically acceptable salt, solvate, or prodrug thereof. In some embodiments, the psychedelic drug mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, or any range therebetween, based on a total weight of the psychedelic drug mixture, of (ii) 5-MeO-DMT-d₉, i.e., one or more of 2-(5-methoxy-1H-indol-3-yl)-N,N-bis(methyl-d₃)ethan-1-amine-1,2,2-d₃ and 2-(5-methoxy-1H-indol-3-yl)-N,N-bis(methyl-d₃)ethan-1-amine-1,1,2-d₃, or a pharmaceutically acceptable salt, stereoisomer, solvate, or prodrug thereof. In some embodiments, the psychedelic drug mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the psychedelic drug mixture, of (iii) 5-MeO-DMT-d₈, i.e., one or more of 2-(5-methoxy-1H-indol-3-yl)-N,N-bis(methyl-d₃)ethan-1-amine-1,1-d₂, 2-(5-methoxy-1H-indol-3-yl)-N,N-bis(methyl-d₃)ethan-1-amine-2,2-d₂, and 2-(5-methoxy-1H-indol-3-yl)-N,N-bis(methyl-d₃)ethan-1-amine-1,2-d₂, or a pharmaceutically acceptable salt, stereoisomer, solvate, or prodrug thereof. In some embodiments, the psychedelic drug mixture consists of or consists essentially of (i) 5-MeO-DMT-d₁₀, i.e., 2-(5-methoxy-1H-indol-3-yl)-N,N-bis(methyl-d₃)ethan-1-amine-1,1,2,2-d₄, or a pharmaceutically acceptable salt, solvate, or prodrug thereof; and (ii) 5-MeO-DMT-d₉, i.e., one or more of 2-(5-methoxy-1H-indol-3-yl)-N,N-bis(methyl-d₃)ethan-1-amine-1,2,2-d₃ and 2-(5-methoxy-1H-indol-3-yl)-N,N-bis(methyl-d₃)ethan-1-amine-1,1,2-d₃, or a pharmaceutically acceptable salt, stereoisomer, solvate, or prodrug thereof.

In some embodiments, the pharmaceutical composition comprises a psychedelic drug mixture comprising: (i) 5-MeO-DMT-d₁₃, i.e., 2-(5-(methoxy-d₃)-1H-indol-3-yl)-N,N-bis(methyl-d₃)ethan-1-amine-1,1,2,2-d₄, or a pharmaceutically acceptable salt, solvate, or prodrug thereof; (ii) 5-MeO-DMT-d₁₂, i.e., one or more of 2-(5-(methoxy-d₃)-1H-indol-3-yl)-N,N-bis(methyl-d₃)ethan-1-amine-1,2,2-d₃ and 2-(5-(methoxy-d₃)-1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) 5-MeO-DMT-d₁₁, i.e., one or more of 2-(5-(methoxy-d₃)-1H-indol-3-yl)-N,N-bis(methyl-d₃)ethan-1-amine-1,1-d₂, 2-(5-(methoxy-d₃)-1H-indol-3-yl)-N,N-bis(methyl-d₃)ethan-1-amine-2,2-d₂, and 2-(5-(methoxy-d₃)-1H-indol-3-yl)-N,N-bis(methyl-d₃)ethan-1-amine-1,2-d₂, or a pharmaceutically acceptable salt, stereoisomer, solvate, or prodrug thereof. In some embodiments, the psychedelic drug mixture comprises from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, or any range therebetween, based on a total weight of the psychedelic drug mixture, of (i) 5-MeO-DMT-d₁₃, i.e., 2-(5-(methoxy-d₃)-1H-indol-3-yl)-N,N-bis(methyl-d₃)ethan-1-amine-1,1,2,2-d₄, or a pharmaceutically acceptable salt, solvate, or prodrug thereof. In some embodiments, the psychedelic drug mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, or any range therebetween, based on a total weight of the psychedelic drug mixture, of (ii) 5-MeO-DMT-d₁₂, i.e., one or more of 2-(5-(methoxy-d₃)-1H-indol-3-yl)-N,N-bis(methyl-d₃)ethan-1-amine-1,2,2-d₃ and 2-(5-(methoxy-d₃)-1H-indol-3-yl)-N,N-bis(methyl-d₃)ethan-1-amine-1,1,2-d₃, or a pharmaceutically acceptable salt, stereoisomer, solvate, or prodrug thereof. In some embodiments, the psychedelic drug mixture comprises, in sum, from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the psychedelic drug mixture, of (iii) 5-MeO-DMT-d₁₁, i.e., one or more of 2-(5-(methoxy-d₃)-1H-indol-3-yl)-N,N-bis(methyl-d₃)ethan-1-amine-1,1-d₂, 2-(5-(methoxy-d₃)-1H-indol-3-yl)-N,N-bis(methyl-d₃)ethan-1-amine-2,2-d₂, and 2-(5-(methoxy-d₃)-1H-indol-3-yl)-N,N-bis(methyl-d₃)ethan-1-amine-1,2-d₂, or a pharmaceutically acceptable salt, stereoisomer, solvate, or prodrug thereof. In some embodiments, the psychedelic drug mixture consists of or consists essentially of (i) 5-MeO-DMT-d₁₃, i.e., 2-(5-(methoxy-d₃)-1H-indol-3-yl)-N,N-bis(methyl-d₃)ethan-1-amine-1,1,2,2-d₄, or a pharmaceutically acceptable salt, solvate, or prodrug thereof; and (ii) 5-MeO-DMT-d₁₂, i.e., one or more of 2-(5-(methoxy-d₃)-1H-indol-3-yl)-N,N-bis(methyl-d₃)ethan-1-amine-1,2,2-d₃ and 2-(5-(methoxy-d₃)-1H-indol-3-yl)-N,N-bis(methyl-d₃)ethan-1-amine-1,1,2-d₃, or a pharmaceutically acceptable salt, stereoisomer, solvate, or prodrug thereof.

In some embodiments, the pharmaceutical composition comprises a psychedelic drug mixture comprising: (i) 5-MeO-DMT-d₅, i.e., one or more of 2-(5-(methoxy-d₃)-1H-indol-3-yl)-N,N-dimethylethan-1-amine-1,1-d₂ and 2-(5-(methoxy-d₃)-1H-indol-3-yl)-N,N-dimethylethan-1-amine-2,2-d₂, or a pharmaceutically acceptable salt, solvate, or prodrug thereof; (ii) 5-MeO-DMT-d₄, i.e., one or more of 2-(5-(methoxy-d₃)-1H-indol-3-yl)-N,N-dimethylethan-1-amine-i-d and 2-(5-(methoxy-d₃)-1H-indol-3-yl)-N,N-dimethylethan-1-amine-2-d, or a pharmaceutically acceptable salt, stereoisomer, solvate, or prodrug thereof; and optionally (iii) 5-MeO-DMT-d₃, i.e., 2-(5-(methoxy-d₃)-1H-indol-3-yl)-N,N-dimethylethan-1-amine, or a pharmaceutically acceptable salt, solvate, or prodrug thereof. In some embodiments, the psychedelic drug mixture comprises, in sum, from 60% to 99% by weight, from 60% to 98% by weight, from 65% to 97% by weight, from 70% to 96% by weight, from 75% to 95% by weight, from 80% to 94% by weight, from 85% to 93% by weight, from 90% to 92% by weight, or any range therebetween, based on a total weight of the psychedelic drug mixture, of (i) 5-MeO-DMT-d₅, i.e., one or more of 2-(5-(methoxy-d₃)-1H-indol-3-yl)-N,N-dimethylethan-1-amine-1,1-d₂ and 2-(5-(methoxy-d₃)-1H-indol-3-yl)-N,N-dimethylethan-1-amine-2,2-d₂, or a pharmaceutically acceptable salt, solvate, or prodrug thereof. In some embodiments, the psychedelic drug mixture comprises, in sum, from 1% to 40% by weight, from 2% to 40% by weight, from 3% to 35% by weight, from 4% to 30% by weight, from 5% to 25% by weight, from 6% to 20% by weight, from 7% to 15% by weight, from 8% to 10% by weight, or any range therebetween, based on a total weight of the psychedelic drug mixture, of (ii) 5-MeO-DMT-d₄, i.e., one or more of 2-(5-(methoxy-d₃)-1H-indol-3-yl)-N,N-dimethylethan-1-amine-i-d and 2-(5-(methoxy-d₃)-1H-indol-3-yl)-N,N-dimethylethan-1-amine-2-d, or a pharmaceutically acceptable salt, stereoisomer, solvate, or prodrug thereof. In some embodiments, the psychedelic drug mixture comprises from 0% by weight to less than 10% by weight, less than 5% by weight, less than 3% by weight, less than 2% by weight, less than 1% by weight, less than 0.5% by weight, less than 0.25% by weight, or any range therebetween, based on a total weight of the psychedelic drug mixture, of (iii) 5-MeO-DMT-d₃, i.e., 2-(5-(methoxy-d₃)-1H-indol-3-yl)-N,N-dimethylethan-1-amine, or a pharmaceutically acceptable salt, solvate, or prodrug thereof. In some embodiments, the psychedelic drug mixture consists of or consists essentially of (i) 5-MeO-DMT-d₅, i.e., one or more of 2-(5-(methoxy-d₃)-1H-indol-3-yl)-N,N-dimethylethan-1-amine-1,1-d₂ and 2-(5-(methoxy-d₃)-1H-indol-3-yl)-N,N-dimethylethan-1-amine-2,2-d₂, or a pharmaceutically acceptable salt, solvate, or prodrug thereof; and (ii) 5-MeO-DMT-d₄, i.e., one or more of 2-(5-(methoxy-d₃)-1H-indol-3-yl)-N,N-dimethylethan-1-amine-i-d and 2-(5-(methoxy-d₃)-1H-indol-3-yl)-N,N-dimethylethan-1-amine-2-d, or a pharmaceutically acceptable salt, stereoisomer, solvate, or prodrug thereof.

In some embodiments, each of the two or more psychedelic drugs constituting the psychedelic drug mixture are in the form of pharmaceutically acceptable salts. In some embodiments, each of the two or more psychedelic drugs constituting the psychedelic drug mixture are in the form of fumarate salts. In some embodiments, each of the two or more psychedelic drugs constituting the psychedelic drug mixture are in the form of benzoate salts. In some embodiments, each of the two or more psychedelic drugs constituting the psychedelic drug mixture are in the form of salicylate salts. In some embodiments, each of the two or more psychedelic drugs constituting the psychedelic drug mixture are in the form of succinate salts.

The psychedelic drug may be present in the pharmaceutical composition in enantiomerically pure form, or as a racemic mixture. As described herein, a racemic active ingredient may contain about 50% of the R- and S-stereoisomers based on a molar ratio (about 48 to about 52 mol %, or about a 1:1 ratio)) of one of the isomers. In some embodiments, the pharmaceutical composition may be provided by combining separately produced compounds of the R- and S-stereoisomers in an approximately equal molar ratio (e.g., about 48 to 52%). In some embodiments, the pharmaceutical composition may contain a mixture of separate compounds of the R- and S-stereoisomers in different ratios. In some embodiments, the pharmaceutical composition contains an excess (greater than 50%) of the R-enantiomer. Suitable molar ratios of R/S may be from about 1.5:1, 2:1, 3:1, 4:1, 5:1, 10:1, or higher. In some embodiments, the pharmaceutical composition may contain an excess of the S-enantiomer, with the ratios provided for R/S reversed. Other suitable amounts of R/S may be selected. For example, the R-enantiomer may be enriched, e.g., may be present in amounts of at least about 55% to 100%, or at least 65%, at least 75%, at least 80%, at least 85%, at least 90%, about 95%, about 98%, or 100%. In some embodiments, the S-enantiomer may be enriched, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included.

The pharmaceutical composition may be formulated with one or more crystalline forms of the psychedelic drug, including one or more crystalline polymorphs. In some embodiments, the pharmaceutical composition includes a mixture of crystalline polymorphs. In some embodiments, the pharmaceutical composition includes a single crystalline polymorph. The pharmaceutical composition may be formulated with one or more amorphous forms of the psychedelic drug, including one or more amorphic polymorphs. In some embodiments, the pharmaceutical composition includes a mixture of amorphous polymorphs. In some embodiments, the pharmaceutical composition includes a single amorphous polymorph. In some embodiments, the pharmaceutical composition includes a mixture of crystalline and amorphous polymorphs. In some embodiments, the pharmaceutical composition comprises a highly pure crystalline form of a psychedelic drug. For example, the pharmaceutical composition may comprise a psychedelic drug, wherein at least 90%, at least 95%, at least 99%, or at least 99.5% by weight of the psychedelic drug present in the pharmaceutical composition is in crystalline form, e.g., as determined by X-ray powder diffraction and/or DSC.

“Pharmaceutically acceptable excipients” may be excipients approved by a regulatory agency of the Federal or a state government or listed in the U.S. Pharmacopeia or other generally recognized pharmacopeia for use in mammals, such as humans. The term “excipient” herein refers to a vehicle, diluent, adjuvant, carrier, or any other auxiliary or supporting ingredient with which the psychedelic drug of the present disclosure is formulated for administration to a mammal. Such pharmaceutically acceptable excipients can be liquids, such as water and oils, including those of petroleum, animal, vegetable or synthetic origin, such as peanut oil, soybean oil, mineral oil, sesame oil and the like. The pharmaceutically acceptable excipients can be water, saline, amino acids or peptides (e.g., leucine), sugars (e.g., lactose, trehalose, pullulan), sugar alcohols (e.g., mannitol), lipids (e.g., phospholipids), magnesium stearate, biodegradable polymers (e.g., polylactic-co-glycolic acid, chitosan, etc.), and the like. The pharmaceutically acceptable excipients can include one or more gases, e.g., to act as a carrier for administration via inhalation. In addition, auxiliary, stabilizers, thickening or viscosity building agents, lubricants, flavoring or taste masking agents, coloring agents, and other pharmaceutical additives may be included in the disclosed compositions, for example those set forth hereinafter. In some embodiments, the pharmaceutical acceptable excipient is a carrier useful for administration via inhalation. In some embodiments, the pharmaceutically acceptable excipient is an aerosol carrier described herein.

In some embodiments, the pharmaceutical composition contains 0.1 to 99.9999 wt. %, preferably 1 to 99.999 wt. %, preferably 5 to 99.99 wt. %, preferably 10 to 99.9 wt. %, preferably 15 to 99 wt. %, preferably 20 to 90 wt. %, preferably 30 to 85 wt. %, preferably 40 to 80 wt. %, preferably 50 to 75 wt. %, preferably 60 to 70 wt. % of the pharmaceutically acceptable excipient relative to a total weight of the pharmaceutical composition.

The pharmaceutical compositions intended for inhalation administration may include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers including surfactants and co-solvents, isotonic agents, buffering agents, antioxidants, local anesthetics, suspending and dispersing agents, wetting or emulsifying agents, complexing agents, sequestering or chelating agents, lyoprotectants or cryoprotectants, thickening agents or viscosity building agents, pH adjusting agents, and inert gases.

Suitable aqueous vehicles include, but are not limited to, water, saline, physiological saline or phosphate buffered saline (PBS), sodium chloride, Ringers solution, isotonic dextrose solution, sterile water, dextrose and lactated Ringers solution. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, acetate, phosphate and citrate buffers. Suitable antioxidants include, but are not limited to, bisulfite and sodium metabisulfite. Suitable local anesthetics include, but are not limited to, procaine hydrochloride. Suitable suspending and dispersing agents include, but are not limited to, sodium carboxymethylcelluose, hydroxypropyl methylcellulose, and polyvinylpyrrolidone. Suitable emulsifying agents include, but are not limited to, polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including ca-cyclodextrin, β-cyclodextrin, hydroxypropyl-3-cyclodextrin, sulfobutylether-β-cyclodextrin, and sulfobutylether 7-O-cyclodextrin (CAPTISOL®, CyDex, Lenexa, Kans.).

Suitable stabilizers include, but are not limited to, organic acids (e.g., citric acid), fatty acids, fatty alcohols, alcohols, long chain fatty acid esters, long chain ethers, hydrophilic derivatives of fatty acids, polyvinylpyrrolidones, polyvinyl ethers, polyvinyl alcohols, hydrocarbons, hydrophobic polymers, moisture-absorbing polymers, glycerol, methionine, monothioglycerol, ascorbic acid, polysorbate, arginine, cyclodextrins, microcrystalline cellulose, modified celluloses (e.g., carboxymethylcellulose, sodium salt), sorbitol, and cellulose gel.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof.

Suitable flavoring agents or taste masking agents include, but are not limited to, natural flavors extracted from plants, such as fruits, and synthetic blends of compounds which produce a pleasant taste sensation, such as peppermint and methyl salicylate, and sweetening agents such as sucrose, lactose, mannitol, syrups, glycerin, and artificial sweeteners, such as saccharin and aspartame.

Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Suitable lyoprotectants or cryoprotectants include, but are not limited to, disaccharides such as sucrose and trehalose, glycols such as ethylene glycol, propylene glycol, and glycerol, anionic polymers such as sulfobutylether-β-cyclodextrin (SBECD) and hyaluronic acid, and hydroxylated cyclodextrins.

The pharmaceutical compositions intended for inhalation administration can be delivered as an aerosol, preferably a mist, with or without a carrier gas, such as air, oxygen, a mixture of helium and oxygen, or other gases and gas mixtures including those containing nitrous oxide, xenon, and/or argon.

The pharmaceutical compositions may also be formulated as a dry powder for insufflation, alone or in combination with an inert carrier such as amino acids or peptides (e.g., leucine), sugars (e.g., lactose, trehalose, pullulan), sugar alcohols (e.g., mannitol), lipids (e.g., phospholipids), magnesium stearate, and biodegradable polymers (e.g., polylactic-co-glycolic acid, chitosan, etc.).

The pharmaceutical compositions disclosed herein may be formulated for single or multiple dosage administration. The single dosage formulations are packaged in an ampule, a vial, container, or canister. The multiple dosage parenteral formulations contain an antimicrobial agent at bacteriostatic or fungistatic concentrations.

In some embodiments, the pharmaceutical compositions are disclosed as ready-to-use sterile solutions. In some embodiments, the pharmaceutical compositions are disclosed as sterile dry soluble products, including lyophilized powders and hypodermic tablets, to be reconstituted with a vehicle prior to use. In some embodiments, the pharmaceutical compositions are disclosed as ready-to-use sterile suspensions. In some embodiments, the pharmaceutical compositions are disclosed as sterile dry insoluble products to be reconstituted with a vehicle prior to use. In some embodiments, the pharmaceutical compositions are disclosed as ready-to-use sterile emulsions.

The pharmaceutical compositions may be in the form of an aerosol or solution for delivery using a pressurized container, pump, spray, atomizer, such as an atomizer using electrohydrodynamics to produce a fine mist, or nebulizer, alone or in combination with a suitable propellant, such as dichlorodifluoromethane, trichlorofluoromethane, dichlorotetrafluoroethane, a hydrofluoroalkane such as 1,1,1,2-tetrafluoroethane (HFA 134A) and 1,1,1,2,3,3,3-heptafluoropropane (HFA 227), carbon dioxide, perfluorinated hydrocarbons such as perflubron, and other suitable gases.

Aqueous solutions suitable for inhalation use can be prepared by dissolving the active psychedelic drug in water, optionally with other aqueous compatible excipients/co-solvents, for example citric acid buffer. Suitable stabilizers and thickening agents can also be added. Aqueous emulsions suitable for inhalation use can be made by dispersing the liquid psychedelic drug in water with viscous material, such as natural or synthetic gums, resins, methylcellulose, sodium carboxymethylcellulose, and other suspending agents.

Solutions or suspensions for use in a pressurized container, pump, spray, atomizer, or nebulizer may be formulated to contain a surfactant or other appropriate co-solvent, or a suitable alternative agent for dispersing, solubilizing, or extending release of the active ingredient(s) disclosed herein, and optionally a propellant. Such surfactants or co-solvents may include, but are not limited to, Polysorbate 20, 60, and 80; Pluronic F-68, F-84, and P-103; cyclodextrin; polyoxyl 35 castor oil; sorbitan trioleate; oleic acid; and an oligolactic acid. Surfactants and co-solvents are typically employed at a level between about 0.01% and about 2% by weight of the pharmaceutical composition. Viscosity greater than that of simple aqueous solutions may be desirable in some cases to decrease variability in dispensing the formulations, to decrease physical separation of components of an emulsion of formulation, and/or otherwise to improve the formulation. Such thickening or viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents, when desirable, are typically employed at a level between about 0.01% and about 2% by weight of the pharmaceutical composition.

Psychedelic drugs can also be dissolved in organic solvents or aqueous mixtures of organic solvents. Organic solvents can be, for example, acetonitrile, chlorobenzene, chloroform, cyclohexane, 1,2-dichloromethane, dichloromethane, 1,2-dimethoxyethane, N,N-dimethylacetamide, N,N-dimethylformamide, 1,4-dioxane, 2-ethoxyethanol, ethylene glycol, formamide, hexane, methanol, ethanol, 2-methoxyethanol, methybutylketone, methylcyclohexane, N-methylpyrrolidone, nitromethane, pyridine, sulfolane, tetralin, toluene, 1,1,2-trichloroethylene, or xylene, and like, including combinations thereof. Organic solvents can belong to functional group categories such as ester solvents, ketone solvents, alcohol solvents, amide solvents, ether solvents, hydrocarbon solvents, etc. each of which can be used.

The pharmaceutical composition may also be formulated as a dry powder for inhalation administration, for example, via a dry powder inhalator (DPI). Here, the psychedelic drug(s) itself can form the powder or the powder can be formed from a pharmaceutically acceptable excipient or carrier and the psychedelic drug(s) is releasably bound to a surface of the carrier powder such that upon inhalation, the moisture in the lungs releases the active ingredient(s) from the surface to make the drug available for systemic absorption. Examples of carrier particles include, but are not limited to, amino acids or peptides (e.g., leucine), sugars (e.g., lactose, trehalose, pullulan), sugar alcohols (e.g., mannitol), lipids (e.g., phospholipids), magnesium stearate, and biodegradable polymers (e.g., polylactic-co-glycolic acid, chitosan, etc.). Particular mention is made to α-lactose monohydrate.

### Aerosols

Devices used to deliver therapeutic agents as described herein as aerosols can be based on, for example, nebulizers, pressurized metered-dose inhaler (pMDI), and dry powder inhalers (DPIs). Pulmonary drug delivery is a form of drug targeting, whether to the site of action in the lungs for topically acting drugs, or the site of absorption for systemically acting drugs. For the former, the advantages of pulmonary delivery include the possibility to use a relatively low dose, a low incidence of systemic side effects and for some drugs a rapid onset of action. For systemically acting drugs, pulmonary delivery offers an opportunity to avoid oral administration and detrimental reactivity within the GI tract, or to avoid injections for drugs that are not well absorbed via the GI tract, along with the possibility for more advantageous pharmacokinetic profiles. The pulmonary epithelium, consisting of an area >100 m², and having an epithelial cell layer<1 μm in thickness, is an attractive target site for systemically acting drugs.

Delivering drugs by inhalation, however, can be relatively complex, for two main reasons. First, the respiratory tract has evolved defense mechanisms that are intended to keep inhaled materials out of the lungs, as well as removing or inactivating them once they have been deposited. Second, it is necessary for a patient to use an inhaler device, and to use it correctly. Failure to adhere to inhaled treatment regimens and misuse of delivery devices are common problems. These issues pose major challenges to the pharmaceutical industry and to healthcare professionals. The major problems with the use of inhalation to deliver drugs are the deposition of aerosolized particles in the oropharyngeal region and upper airways and the lack of coordination between the device activation and inhalation due to lack of patient training.

Devices for inhaled drug delivery have two basic functions, namely aerosol formation and facilitation of aerosol transport into the lungs. A distinction is made between passive and active devices. A passive device derives the energy required for aerosol formation from the inhaled air stream, i.e., from the patient, while active devices create the aerosol independently of the patient's inhalation. Inhalation devices can be further categorized in various ways, such as single-dose versus multi-dose, or disposable versus reusable. Multi-dose devices can provide benefits for chronic therapy, such as cost reduction, portability, ease of use, and convenience. For irregular administrations and one-time applications, disposable devices may be more suitable. Furthermore, aspects such as the risk of device contamination acting as a reservoir for microbial growth and allowing the development of antibiotic resistance may affect the choice for a multi- or single-dose device.

Three main types of inhalation devices can be used for pulmonary delivery: pressurized metered dose inhalers (pMDIs), nebulizers, soft mist inhalers, and dry powder inhalers (DPIs).

In general, pMDIs generate aerosol faster than the patient can inhale. Coordination between device actuation and patient inhalation is especially difficult in children and the elderly. With some DPIs, it is required that the patient inhales at maximum force to disperse then inhale the powder, which unless properly trained, is rarely achieved. In these scenarios, most of the aerosol deposits in the upper airways. For pMDIs, this problem can be addressed by providing a spacer or by designing a breath-activated inhaler instead of breath-coordinated devices.

The effectiveness of pulmonary delivery is also dependent upon the breathing pattern of the patient. Rapid inspiration is not recommended when using pMDIs and nebulizers, since it creates a turbulent air flow and fast velocity which increases the deposition by impaction in the upper airways. However, rapid inspiratory air flow is required to deagglomerate drug particles for inhalation in DPI devices.

There are two main types of nebulizers, jet and ultrasonic, that differ in the force used to generate the aerosol from the respective liquid. Depending on the type, nebulizers can generate 1-5 μm droplets. Nebulizers do not require patient coordination between inhalation and actuation, thus they are useful for pediatric, elderly, ventilated, non-conscious patients, or those who are unable to use pMDIs or DPIs. Nebulizers have the capability of delivering larger doses compared to the other aerosol devices even though this will require longer administration times

Nebulizer devices can be breath-enhanced, breath-actuated, and vibrating mesh nebulizers. The design of breath-enhanced jet nebulizers is modified to allow for air entrainment during inspiration and to vent the expired air outside of the device. The main advantage of this approach is to increase the output rate, which in turn will decrease the administration time.

Breath-actuated nebulizers emit aerosolized droplets only when the patient inhales. Therefore, no drug is wasted during exhalation as the case of regular jet nebulizers and dissemination of expensive or toxic drugs to the surrounding environment is avoided.

Vibrating mesh nebulizers have a mesh plate that, when it vibrates through the action of the piezoelectric element, breaks the liquid into very fine droplets, which increases the volume of aerosol deposited in the alveoli. Vibrating mesh nebulizers can have an electronic indicator that show when the patient is breathing appropriately and only then, it releases the dose, with droplet size of mass median aerodynamic diameter, 4 μm and minimum drug loss (˜1%). Smart devices can include a vibrating mesh nebulizer coupled with an adaptive aerosol delivery software that adjusts the aerosol emission based on the breathing pattern of the patient, which reduces drug loss and increases the inhaled mass. Such a device can adjust the dose delivery based on patient's last three breaths and provide feedback after dose delivery.

Soft Mist Inhaler (SMI) inhalation devices can be used to deliver the compositions described herein. The SMI is a nebulizer, as it disperses a solution of the active agent into fine droplets. It differs from a traditional nebulizer in that it is a hand-held, portable device that does not require an external power source, but is actuated by a mechanical spring. The instantaneous formation of the aerosol is comparable to a pMDI; thus, proper actuation-inhalation coordination is necessary. While it generally takes longer before the entire aerosol is generated (1.5 s versus 0.21-0.36 s for an HFA-pMDI) and the aerosol is emitted as a slow-moving mist, this allows for a relatively high lung deposition.

In some embodiments, methods of delivering psychedelic drugs by aerosol inhalation are provided. An aerosol, such as a mist, can be delivered using air, oxygen, and/or oxygen and helium mixtures as a carrier gas. The air, oxygen, and/or oxygen and helium mixture can be delivered at room temperature or heated. In some embodiments, an aerosol, such as a mist comprising a psychedelic drug is delivered via inhalation using heated helium-oxygen (heliox) mixtures. Due to very low viscosity of helium the helium-oxygen mixtures generate gaseous streams characterized by laminar flow that is a highly desirable feature for reaching out into the deep lung areas and reducing deposition of the drug in the respiratory tract, one of the major obstacles in dose delivery via inhalation. A patient can inhale a dissolved free-base or salt formulation of a psychedelic drug as a mist into an alveolar region of the patient's lungs. The psychedelic drug or derivative can be delivered to a fluid lining of the alveolar region of the lungs and can be systemically absorbed into patient blood circulation. Advantageously, these formulations can be effectively delivered to the blood stream upon inhalation to the alveolar regions of the lungs.

Devices suitable for delivery of heated or unheated air, oxygen, or helium-oxygen mixtures include, for example, continuous mode nebulizers Flo-Mist (Phillips) and Hope (B&B Medical Technologies) and the accessories such as regulators, e.g., Medipure™ Heliox-LCQ System (PraxAir) and control box, e.g., Precision Control Flow (PraxAir). In another embodiment, a full delivery setup can be a device as described in, for example, Russian patent RU199823U1.

The term “heliox” as used herein refers to breathing gas mixtures of helium gas (He) and oxygen gas (O₂). In some embodiments, the heliox mixture can contain helium in the mixture of helium and oxygen at about 50%, 60%, 70%, 80% or 90% by volume and contain oxygen in the mixture of helium and oxygen at about 50%, 40%, 30%, 20%, or 10% by volume. The heliox mixture can thus contain helium and oxygen in a 50:50, 60:40, 70:30, 80:20, 90:10 ratio. In some embodiments, heliox can generate less airway resistance through increased tendency to laminar flow and reduced resistance in turbulent flow.

The use of heat in heliox mixtures can further enhance drug delivery by increasing permeability of key physical barriers for drug absorption. Heating of mucosal surfaces can increase permeability by enhancing peripheral blood circulation and relaxing the interstitial junction, as well as other mechanisms. Helium has a thermal conductivity almost 10 times higher than oxygen and nitrogen and can facilitate heat transfer more efficiently. A dry heliox mixture can be used safely as a pretreatment step when warmed up to as high as 110° C. (e.g., heated to about 70, 80, 90, 100, or 110° C.), which can enable the dry heliox mixture to heat mucosal surfaces of the lung and respiratory tract more efficiently.

Various types of personal vaporizers can be used to deliver the therapeutic compositions described herein and are known in the art. In general, personal vaporizers are characterized by heating a solid drug or compound. Vaporizers can work by directly heating a solid drug or compound to a smoldering point. Vaporizing a solid or solid concentrate can be done by convection on conduction. Convection heating of solid concentrate involves a heating element coming into contact with water, or another liquid, which then vaporizes. The hot vapor in turn directly heats the solid or solid concentrate to a smoldering point, releasing a vapor to be inhaled by a user. Conduction heating involves direct contact between the solid or solid concentrate and the heating element, which brings the solid to a smoldering point, releasing vapor to be inhaled by a user. Though vaporizers present advantages over smoking in terms of lung damage, the drug/active agent that is vaporized can be substantially deteriorated by the vaporizing heat.

A vapor is a solid substance in the gas phase at a temperature lower than its critical temperature, meaning that the vapor can be condensed to a liquid by increasing the pressure on it without reducing the temperature.

An aerosol, as used herein, is a suspension of fine solid particles or liquid droplets in a gas phase (e.g., air, oxygen, helium, nitrous oxide, xenon, argon, and other gases, as well as mixtures thereof). A mist, as used herein, is a subset of aerosols, differing from a vapor, and is a dispersion of liquid droplets (liquid phase) suspended in the gas phase (e.g., air, oxygen, helium, and mixtures thereof). The liquid droplets of a mist can comprise a psychedelic drug dissolved in an aqueous liquid or organic solvent. The liquid phase of mist droplets can contain thousands or millions of molecules. The gas phase of a mist can comprise air, oxygen, helium, other gases, and mixtures thereof. Mists do not comprise solid particulates. Mists can be created by any suitable methods, including for example, use of an inhaler or nebulizer.

In some embodiments, a psychedelic drug is delivered via a nebulizer, which generates an aqueous-droplet aerosol, such as a mist, containing the psychedelic drug, which is optionally combined with a heated helium-oxygen mixture. In some embodiments, the psychedelic drug is delivered via a nebulizer, which generates an aqueous-droplet aerosol, such as a mist, containing the psychedelic drug, which is combined with nitrous oxide (or a noble gas such as xenon and/or argon) or a gas mixture containing nitrous oxide (or noble gas), such as a nitrous oxide (or noble gas)-air or nitrous oxide (or noble gas)-oxygen mixture. The nitrous oxide, xenon, and/or argon (being NMDA receptor antagonists) can augment the effect of the psychedelic drug and provide the ability to use less psychedelic drug to obtain similar levels of effect.

For example, a preparation of a psychedelic drug can be placed into a liquid medium and put into an aerosol by a device, such as a nebulizer. In some embodiments, a nebulizer can be, for example, a pneumatic compressor nebulizer, an ultrasonic nebulizer, a vibrating mesh or horn nebulizer, or a microprocessor-controlled breath-actuated nebulizer. In another embodiment, a nebulizer device can be a device as described in, for example, Russian patent RU199823U1.

A nebulizer is a device that turns a drug, such as a psychedelic drug, in solution or suspension into a fine aerosol, such as a mist, for delivery to the lungs. A nebulizer can also be referred to as an atomizer. To atomize is to put a dissolved or dispersed drug into an aerosol, such as a mist, form. To deliver a drug by nebulization, a drug can be dispersed in a liquid medium, for example, water, ethanol, or propylene glycol, with optional excipients. Additionally, psychedelic drugs can be carried in a vehicle such as, for example liposomes, polymers, emulsions, micelles, nanoparticles, or polyethyleneimine (PEI). Liquid drug formations for nebulizers can be, for example, aqueous solutions or viscous solutions. After application of a dispersing forcer (e.g., jet of gas, ultrasonic waves, or vibration of mesh), the dissolved psychedelic drug is contained within liquid droplets, which are then inhaled. A mist can comprise liquid droplets containing the drug in air or another gaseous mixture (e.g., a mixture of helium and oxygen).

Jet nebulizers (also known as pneumatic nebulizers or compressor nebulizers) use compressed gas to make a mist. In some embodiments, a jet nebulizer is a microprocessor-controlled breath-actuated nebulizer, also called a breath-actuated nebulizer. A breath-actuated nebulizer creates a mist only when a patient is inhaling, rather than creating a mist continuously. A mist can be generated by, for example, passing air flow through a Venturi in a nebulizer bowl or cup. A Venturi is a system for speeding the flow of a fluid by constricting fluid in a cone shape tube. In the restriction, the fluid must increase its velocity, thereby reducing its pressure and producing a partial vacuum. As the fluid exits the constriction point, its pressure increases back to the ambient or pipe level pressure. This can form a low-pressure zone that pulls up droplets through a feed tube from a solution of drug in a nebulizer bowl, and in turn this creates a stream of atomized droplets, which flow to a mouthpiece. Higher air flows lead to a decrease in particle size and an increase in output. Due to droplets and solvent that saturates the outgoing gas, jet nebulizers can cool a drug solution in the nebulizer and increase solute concentration in the residual volume. A baffle in a nebulizer bowl or cup can be impacted by larger particles, retaining and returning them to the solution in the nebulizer bowl or cup to be reatomized. Entrainment of air through a nebulizer bowl as the subject inhales can increase mist output during inspiration. Generation of a mist can occur with a smaller particle size distribution, but using smaller particle sizes can result in an increased nebulization time.

The unit of measurement generally used for droplet size is mass median diameter (MMD), which is defined as the average droplet diameter by mass. This unit can also be referred to as the mass mean aerodynamic diameter, or MMAD. The MMD droplet size for jet nebulizers can be about 1.0, 1.5, 2.0, 2.5, 3.0, 3.5, 4.0, 5.0, 6.0, 7.0, 8.0, 9.0, 10.0 μm or more (or any range between about 1.0 and 10.0 μm), which can be smaller than that of ultrasonic nebulizers.

Ultrasonic nebulizers generate mists by using the vibration of a piezoelectric crystal, which converts alternating current to high-frequency (about 1 to about 3 MHz) acoustic energy. The solution breaks up into droplets at the surface, and the resulting mist is drawn out of the device by the patient's inhalation or pushed out by gas flow through the device generated by a small compressor. Ultrasonic nebulizers can include large-volume ultrasonic nebulizers and small-volume ultrasonic nebulizers. Droplet sizes tend to be larger with ultrasonic nebulizers than with jet nebulizers. The MMD droplet size for ultrasonic nebulizers can be about 2.0, 2.5, 3.0, 3.5, 4.0, 4.5, 5.0, 5.5, 6.0, 6.5, 7.0, 7.5, 8.0, 9.0, 10.0 μm or more (or any range between about 2.0 and 10.0 μm). Ultrasonic nebulizers can create a dense mist, with droplets at about 100, 150, 200, 250, 300 μm/L or more.

Mesh nebulizer devices use the vibration of a piezoelectric crystal to indirectly generate a mist. Mesh nebulizers include, for example, active mesh nebulizers and passive mesh nebulizers. Active mesh nebulizers use a piezo element that contracts and expands on application of an electric current and vibrates a precisely drilled mesh in contact with the drug solution to generate a mist. The vibration of a piezoelectric crystal can be used to vibrate a thin metal plate perforated by several thousand holes. One side of the plate is in contact with the liquid to be atomized, and the vibration forces this liquid through the holes, generating a mist of tiny droplets. Passive mesh nebulizers use a transducer horn that induces passive vibrations in the perforated plate with tapered holes to produce a mist. Examples of active mesh nebulizers include the Aeroneb® (Aerogen, Galway, Ireland) and the eFlow® (PARI, Starnberg, Germany), while the Microair NE-U22 ® (Omron, Bannockburn, IL) is a passive mesh nebulizer. Mesh nebulizers are precise and customizable. By altering the pore size of the mesh, the device can be tailored for use with drug solutions of different viscosities, and the output rate changed. Use of this method of atomization can offer several advantages. The size of the droplets can be extremely precise because droplet size can be determined by the size of the holes in the mesh (which may be tailor-made to suit the application). Nebulizer meshes can be manufactured using methods such as electrodeposition, electroplating, and laser cutting to produce a liquid particle in gas in the respirable range. Mesh can be made of metal alloy. The metals used in mesh manufacture can include platinum, palladium, nickel, and stainless steel. The size of the droplet is about twice the size of the mesh hole. Mesh holes, therefore, can be about 0.1, 0.5, 1.0, 1.5, 2.0, 2.5, 3.0, 3.5, 4.0, 4.5, 5.0 μm or more (or any value in between about 0.1 and 5.0 μm). Mist generation in mesh nebulizers can vary based on the shape of the mesh, the material that the mesh is made of, and the way that the mesh is created. In other words, different meshes can produce different sized liquid particles suspended in gas. Generally, MMD droplet size for mesh nebulizers can be about 1.0, 1.5, 2.0, 2.5, 3.0, 3.5, 4.0, 4.5, 5.0, 5.5., 6.0, 6.1, 6.2, 6.3, 6.4, 6.5, 6.6, 6.7, 6.8, 6.9, 7.0 μm or more (or any value in between about 1.0 and 7.0 μm).

Additionally, droplet size can be programmable. In particular, geometric changes can be made to a nebulizer to provide a specific desired droplet size. Additionally, droplet size can be controlled independently of droplet velocity. The volume of liquid atomized, and the droplet velocity can also be precisely controlled by adjusting the frequency and amplitude of the mesh vibration. Furthermore, the number of holes in the mesh and their layout on the mesh can be tailored. Mesh nebulizers can be powered either by electricity or by battery.

A mist output rate in standing cloud mL per minute (for any atomization methodology described herein) can range from, for example, 0.1, 0.2, 0.3, 0.4, 0.5, 0.6, 0.7, 0.8, 0.9 mL/minute or more (or any range between about 0.1 and 0.9 mL/minute) and the residual volume in any type of nebulizer reservoir can range from a about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mL or more (or any range between about 0.01 and 2.0 mL). Precise droplet size control can be advantageous since droplet size can correlate directly to kinetic drug release (KDR). Precise control of KDR can be achievable with precise control of droplet size. Psychedelic drugs can be delivered via a mist using any methodology with an MMD droplet size of about 0.5, 1.0, 1.5, 2.0, 2.5, 3.0, 3.5, 4.0, 5.0, 6.0, 7.0, 8.0, 9.0, 10.0 μm or more (or any range between about 0.5 and 10.0 μm).

In some embodiments, a psychedelic drug can be delivered via a continuous positive airway pressure (CPAP) or other pressure-assisted breathing device. A pressure-assisted breathing device forces a continuous column of compressed air or other gas at a fixed designated pressure against the face and nose of the patient, who is wearing a mask or nasal cap. When the patient's glottis opens to inhale, the pressure is transmitted throughout the airway, helping to open it. When the patient exhales, pressure from the deflating lungs and chest wall pushes air out against the continuous pressure, until the two pressures are equal. The air pressure in the airway at the end of exhalation is equal to the external air pressure of the machine, and this helps “splint” the airway open, allowing better oxygenation and airway recruitment. A pressure-assisted breathing device can be coupled with a means for introducing mist particles into the gas flow in the respiratory circuit and or a means for discontinuing the introduction of mist particles into the respiratory circuit when the patient exhales. See, e.g. U.S. Pat. No. 7,267,121.

In another embodiment, a mist can be delivered by a device such as a metered dose inhaler (MDI) (also referred to as a pressurized metered dose inhaler or pMDI), which generates an organic solvent-droplet mist containing the psychedelic drugs, which is optionally combined with a heated helium-oxygen mixture. In some embodiments, a psychedelic drug can be delivered via a metered dose inhaler, MDI. MDI devices can include a canister which contains the psychedelic drug and a propellant, a metering valve which dispenses the medicament from the canister, an actuator body that receives the canister and which forms an opening for oral inhalation, and an actuator stem which receives the drug from the canister and directs it out the opening in the actuator body. Moving the drug canister relative to the actuator body and actuator stem causes the metering valve to release the predetermined amount of the drug. In some embodiments, the psychedelic drug can be dissolved in a liquid propellant mixture (sometimes including small amounts of a volatile organic solvent) stored in a pressurized container of the MDI. The “metered dose” is the dose that is prepackaged in a single-dose inhaler, or which in a multidose inhaler is automatically measured out of a reservoir in preparation for inhalation. MDI devices can be aided with spacers. An MDI spacer is a spacer that goes between the MDI and the mouth of a user of the MDI. An MDI spacer allows droplets in the atomized dose to settle out a bit and mix with air or other gas, thus allowing for more effective delivery of a metered dose into a user's lungs when inhaled. An MDI spacer assists in preventing a user from inhaling the metered dose directly from an MDI where the dose would be traveling so fast that the droplets of the atomized spray from the MDI hit and stick to the back of the user's throat rather than being inhaled into the user's lungs where the drug of the metered dose is designed to be delivered. MDI devices offer the advantage of regular dosing, which can be controlled in the manufacture of the drug.

Drugs can also be delivered by dry powder inhalers (DPI). In such DPI devices, the drug itself can form the powder or the powder can be formed from a pharmaceutically acceptable excipient or carrier and the drug is releasably bound to a surface of the carrier powder such that upon inhalation, the moisture in the lungs releases the drug from the surface to make the drug available for systemic absorption. In some embodiments, the psychedelic drug is delivered by use of a dry powder inhaler (DPI). Depending on the psychedelic drug used, the drug can be formed into the necessary powder itself, or can be releasably bound to a surface of a carrier powder. Such carrier powders are known in the art (see, e.g., H. Hamishehkar, et al., “The Role of Carrier in Dry Powder Inhaler”, DOI:10.5772/51209 (2012).).

DPI is generally formulated as a powder mixture of coarse carrier particles and micronized drug particles with aerodynamic particle diameters of 1-5 μm (Iida et al., “Preparation of dry powder inhalation by surface treatment of lactose carrier particles.” Chem Pharm Bull, 511150009-2363 pubmed.ncbi.nlm.nih.gov/12520118/2003). Carrier particles are often used to improve drug particle flowability, thus improving dosing accuracy and minimizing the dose variability observed with drug formulations alone while making them easier to handle during manufacturing operations. Carrier particles should have several characteristics such as physico-chemical stability, biocompatibility and biodegradability, compatible with the drug substance and must be inert, available and economical. The choice of carrier particle (both content and size) is well within the purview of one of ordinary skill in the art. The most common carrier particles are made of lactose or other sugars, with α-lactose monohydrate being the most common lactose grade used in the inhalation field for such particulate carriers. Solid dosage forms suitable for dry powder inhalation administration may be prepared according to processes known in the art, including, but not limited to, mixing, co-jet milling, liposomal processes, lyophilization, and spray drying.

In some embodiments, any of the delivery devices above can be manufactured with smart technology enabling remote activation of the drug delivery. The remote activation can be performed via computer or mobile app. To ensure security, the remote activation device can be password encoded. This technology enables a healthcare provider to perform telehealth sessions with a patient, during which the healthcare provider can remotely activate and administer the psychedelic drug via the desired delivery device while supervising the patient on the televisit.

In some embodiments, the delivery device is an inhalation delivery device for delivery of a combination of nitrous oxide (or noble gas such as xenon and/or argon) and a psychedelic drug by inhalation by a patient in need thereof, comprising an inhalation outlet portal for administration of the combination of nitrous oxide (or noble gas) and the psychedelic drug to the patient; a container configured to deliver nitrous oxide gas (or noble gas) to the inhalation outlet portal; and a device configured to generate and deliver an aerosol comprising the psychedelic drug to the inhalation outlet portal. In some embodiments, the inhalation outlet portal is selected from a mouthpiece or a mask covering the patient's nose and mouth. In some embodiments, the device configured to generate and deliver the aerosol to the inhalation outlet portal is a nebulizer. In some embodiments, the nebulizer is a jet nebulizer and the nitrous oxide gas (or noble gas) acts as a driving gas for the jet nebulizer. In some embodiments, the device further comprises electronics configured to provide remote activation and operational control of the inhalation delivery device as noted above.

In some embodiments, the device is a dual delivery device configured to administer the psychedelic drug, such as in the form of an aerosol, and to simultaneously administer a controlled amount of nitrous oxide, xenon, and/or argon. Any of the above aerosol delivery devices can be used for such a device, with the addition of a source of nitrous oxide, xenon, and/or argon configured to provide a metered, controlled dose/flow rate of nitrous oxide, xenon, and/or argon through the same administration outlet as the aerosol delivery device. In some embodiments, the driving gas for the nebulization of the psychedelic drug is the nitrous oxide itself, or xenon gas itself, or argon gas itself.

When co-administering the psychedelic drug with nitrous oxide (or noble gas such as xenon and/or argon), the nitrous oxide (or noble gas) can be in the form of a mixture of nitrous oxide (or noble gas) and oxygen (or air), e.g., N₂O (or noble gas) and O₂; N₂O (or noble gas) and air; N₂O (or noble gas) and medical air (medical air being 78% nitrogen, 21% oxygen, 1% other gases); N₂O (or noble gas) and a N₂/O₂ mix; N₂O (or noble gas) and O₂ enriched medical air; N₂O (or noble gas) and a He/O₂ mix etc. Thus, in addition to nitrous oxide (or noble gas) and oxygen, such gas mixtures may further include other gases such as one or more of N₂, Ar, CO₂, Ne, CH₄, He, Kr, H₂, Xe, H₂O (e.g., vapor), etc. Nitrous oxide (or noble gas) may be administered using a blending system that combines N₂O (or noble gas), O₂ and optionally other gases from separate compressed gas cylinders into a gas mixture which is delivered to a patient via inhalation. Alternatively, the gas mixture containing nitrous oxide (or noble gas) may be packaged, for example, in a pressurized tank or in small, pressurized canisters, or handheld devices which are easy to use and/or portable. The blending system, handheld devices, and/or pressurized tanks/canisters may be adapted to fluidly connect to an inhalation device such as a device capable of generating an aerosol of the psychedelic drug. Nitrous oxide (or noble gas) itself, or the gas mixture comprising nitrous oxide (or noble gas) may be used for the generation of the aerosol (i.e., as the gas phase component of the aerosol) or as a carrier gas to facilitate the transfer of a generated aerosol to a patient's lungs. In some embodiments, N₂O (or noble gas, e.g., xenon or argon) is present in the gas mixture at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the gas mixture. For example, the amount of nitrous oxide (or noble gas) may be 15 to 25% by volume of the nitrous oxide (or noble gas)/oxygen (or air) mixture, e.g., about 15 to 20% by volume of the nitrous oxide (or noble gas)/oxygen (or air) mixture.

Advantageously, low levels of nitrous oxide, xenon, and/or argon at a level of about 15-25% by volume (e.g., about 25, 24, 23, 22, 21, 20, 19, 18, 17, 16, 15 or lower), for example about 15-20% by volume, for the about 90, 60, 45, 30, 15 minutes or less can provide good efficacy and with significantly reduced side effect profile. For example, the amount and/or severity of nausea, headache, anxiety, emotional discomfort, confusion, dizziness, and sedation can be reduced when low levels of nitrous oxide, xenon, and/or argon (e.g., a level of about 15-25%) is used. In some embodiments, a mixture of nitrous oxide (or noble gas such as xenon and/or argon) and oxygen (or air) is administered, without the administration of psychedelic drug. In such an embodiment, the mixture of nitrous oxide (or noble gas) and oxygen (or air) contains the nitrous oxide (or noble gas) in an amount of 15 to 25% by volume of the total gas, for example 15 to 20% by volume of the total gas. The time of administration of the gas mixture containing nitrous oxide (or noble gas), e.g., nitrous oxide (or noble gas)/oxygen (or air) mixture, can be any desired duration, for example, 5 minutes, 10 minutes 15 minutes, 20 minutes, 30 minutes, 40 minutes, 45 minutes, 50 minutes, 60 minutes, 90 minutes, 120 minutes, 150 minutes, 180 minutes, or any range therebetween.

In some embodiments, the co-administration of the psychedelic drug with nitrous oxide (or noble gas such as xenon and/or argon), in the form of a gas mixture containing nitrous oxide (or noble gas) (e.g., nitrous oxide (or noble gas)/oxygen (or air) mixture) can reduce the amount of psychedelic drug to be delivered by about 2, 5, 10, 20, 30, 40, 50, 60, 70 percent or more, as compared to a dose not delivered with nitrous oxide (or noble gas) as described herein. The lower amount of psychedelic drug can result in fewer or less severe side effects such as psychological disorders such as acute psychedelic crisis (a bad trip), dysphoric physiological and psychological side effects, nausea, headache, anxiety, emotional discomfort, confusion, dizziness, and sedation.

### Delivery of Psychedelic Drugs and Helium Oxygen Mixtures

Methods disclosed herein provide for systemic delivery of a psychedelic drug. In particular, a psychedelic drug can be delivered to a patient's CNS. Doses can be optimized for individual patients' metabolisms and treatment needs. Larger doses with deleterious or undesirable side-effects can be avoided by using small doses. Methods of treating various central nervous system (CNS) diseases and other conditions are described herein. The methods can comprise delivering a psychedelic drug to a patient in need thereof via inhalation of an aerosol comprising the drug and a gas such as air, oxygen, helium, or a mixture of helium and oxygen (i.e., a heliox mixture). In some embodiments the air, oxygen, helium, or mixture helium and oxygen can be heated. The method can further comprise a using a device containing a balloon with an oxygen-helium mixture equipped with a reducer and a mask connected to each other by a gas or air connecting tube, which contains an additional heating element capable of heating the gas mixture up to 120° C., a nebulizer with a vibrating porous plate or mesh, ensuring the passage of droplets with a size of less than 5 microns through it, and a disinfection unit.

In some embodiments a psychedelic drug is delivered to the lower respiratory tract, for instance, to a pulmonary compartment such as alveoli, alveolar ducts and/or bronchioles. From there, the drug can enter the blood stream and travel to the central nervous system. In some embodiments, delivering a psychedelic drug to a patient in need thereof via inhalation of a mist can deliver the psychedelic drug to the patient's CNS without passing through the liver. Administration via inhalation can allow gaseous drugs or those dispersed in a liquid or a mist, to rapidly deliver the psychedelic drug to the blood stream, bypassing first-pass metabolism. First-pass metabolism, also known as “first-pass effect” or “presystemic metabolism” describes drugs that enter the liver and undergo extensive biotransformation.

In some embodiments, the present disclosure provides a treatment step, in which a psychedelic drug can be administered to a patient in need thereof by administering via inhalation a mixture of helium and oxygen heated to about 50° C., 51° C., 52° C., 53° C., 54° C., 55° C., 56° C., 57° C., 58° C., 59° C., 60° C., or more (or any range between 50° C. to 60° C.) and the atomized psychedelic drug. In some embodiments a mist or vapor of the psychedelic drug can have a particle size from about 0.1 microns to about 10 microns (e.g., about 10, 5, 4, 3, 2, 1, 0.1 or less microns). In some embodiments, the psychedelic drug can be atomized via a nebulizer creating an inhalant that is a mist with the dissolved psychedelic drug. In some embodiments, the atomized psychedelic drug is driven down the patient delivery line by the patient's inhalation. In another embodiment, the atomized psychedelic drug is driven down the patient delivery line by the patient's inhalation using a carrier gas. The carrier gas can be air, oxygen, a mix of oxygen and helium, heated air, heated oxygen, or heated helium and oxygen mixture.

In other embodiments of the present disclosure, the treatment step can be preceded by a pretreatment step. In some embodiments, the pretreatment step can comprise first administering a pretreatment inhalation therapy prior to administration of the mist of the psychedelic drug. In some embodiments, the pretreatment inhalation step can comprise (i) administering via inhalation air, oxygen, or mixture of helium and oxygen heated to about 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., or more (or any range between about 90° C. and 120° C.) and no psychedelic drug, and then (ii) administering a treatment step of inhalation air, oxygen, a mix of oxygen and helium, heated air, heated oxygen, or heated helium and oxygen mixture. Heated air, heated oxygen, or heated helium and oxygen mixture, in combination with the atomized psychedelic drug, can be heated to about 50° C., 51° C., 52° C., 53° C., 54° C., 55° C., 56° C., 57° C., 58° C., 59° C., 60° C., or more (or any range between about 50° C. and 60° C.).

In some embodiments of the present disclosure, step a pretreatment step (i) and a treatment step (ii) can be repeated 0, 1, 2, 3, 4, 5, or more times. In some embodiments of the present disclosure, steps (i) and (ii) can be repeated 0, 1, 2, 3, 4, 5, or more times followed by the treatment step, which can be repeated 0, 1, 2, 3, 4, 5, or more times. In some embodiments of the present disclosure, the treatment step can be repeated 0, 1, 2, 3, 4, 5, or more times with no pretreatment step.

Treatment, with optional pretreatment, can be administered once a week, twice a week, once a day, twice a day, three times a day or more, or other treatment schedules set forth herein. Each treatment can be for about 1, 5, 10, 20, 30, 45, 60, 75, 90 or more minutes, or any range therebetween.

A drug delivery procedure can comprise an inhaled priming no-drug hot heliox mixture to effectively preheat the mucosal bed followed by inhaling an atomized psychedelic drug, again driven by the heated heliox, but at lower temperatures, that are now dictated by lower heat tolerance to the wet vs. dry inhaled gas stream. Consequently, this procedure can be conducted in multiple repeated cycles, wherein a target pharmacokinetic (PK) and drug exposure is controlled by the concentration of the drug, temperature, flow rate of the helium oxygen mixture, composition of the mixture, number and durations of cycles, time and combinations of the above.

Methods of delivery described herein can be used to treat certain diseases and disorders. Treating and treatment refers to methods of alleviating or abrogating a condition, disorder, disease, one or more symptoms of a condition, disorder, or disease, or combinations thereof. Treating or treatment can include partial or complete halting of the progression of the condition, disorder, disease, or partial or complete reversal of the condition, disorder, disease. A treatment can provide a therapeutic benefit such as the eradication or amelioration of one or more of the physiological or psychological symptoms associated with the underlying condition, disease, or disorder such that an improvement is observed in the patient, notwithstanding the fact that the patient may still be affected by the condition.

Therefore, provided herein are methods of treating a central nervous system (CNS) disorder or psychological disorder comprising administering via inhalation a psychedelic drug of the present disclosure, e.g., a heated mixture of helium and oxygen and an atomized psychedelic drug. The treatment can alleviate one or more symptoms of the disorder.

In some embodiments, the psychedelic drug can be administered for treatment of CNS disease or other disorder. In some embodiments, the psychedelic drug can be administered to treat depression including, but not limited to major depression, melancholic depression, atypical depression, or dysthymia. In some embodiments the psychedelic drug can be administered to treat psychological disorders including anxiety disorder, obsessive compulsive disorder, addiction (narcotic addiction, tobacco addiction, opioid addiction), alcoholism, depression and anxiety (chronic or related to diagnosis of a life-threatening or terminal illness), compulsive behavior, or a related symptom.

In some embodiments, the disease or disorder can include central nervous system (CNS) disorders and/or psychological disorders, including, for example, post-traumatic stress disorder (PTSD), major depressive disorder (MDD), treatment-resistant depression (TRD), suicidal ideation and suicide attempts, bipolar and related disorders (including but not limited to bipolar I disorder, bipolar II disorder, cyclothymic disorder), obsessive-compulsive disorder (OCD), generalized anxiety disorder (GAD), acute psychedelic crisis, social anxiety disorder, substance use disorders (including but not limited to alcohol use disorder, opioid use disorder, amphetamine use disorder, nicotine use disorder, and cocaine use disorder), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, gambling disorder, eating disorders such as anorexia nervosa, bulimia nervosa, binge-eating disorder, etc., and paraphilic disorders such as, for example, pedophilic disorder, exhibitionistic disorder, voyeuristic disorder, fetishistic disorder, sexual masochism or sadism disorder, and transvestic disorder, etc., sexual dysfunction, and obesity. In some embodiments, the disease or disorder may include conditions of the autonomic nervous system (ANS). In some embodiments, the disease or disorder may include pulmonary disorders (e.g., asthma and chronic obstructive pulmonary disorder (COPD). In some embodiments, the disease or disorder may include cardiovascular disorders (e.g., atherosclerosis).

In some embodiments, the methods provided herein are used to treat a subject with a depressive disorder. As used herein, the terms “depressive disorder” or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions. In some embodiments, the depressive disorder is major depressive disorder (MDD), atypical depression, bipolar disorder, catatonic depression, depressive disorder due to a medical condition, postpartum depression, premenstrual dysphoric disorder, seasonal affective disorder, or treatment-resistant depression (TRD).

In some embodiments, the disease or disorder is major depressive disorder (MDD). As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by symptoms of depression lasting at least two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all—or almost all—activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide. Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e., depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g., vitamin B12 deficiency), autoimmune conditions (e.g., lupus and rheumatoid arthritis), viral or other infections (hepatitis, mononucleosis, herpes), back pain, and cancer (e.g., pancreatic cancer). In some embodiments, the disease or disorder is cancer related depression and anxiety.

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Mental Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with treatment-resistant depression (TRD). The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, 5 treatment attempts, or more, for example with a conventional antidepressant. In some embodiments, the subject with treatment-resistant depression has been diagnosed with major depressive disorder and has failed to respond to 3 or more treatment attempts. In some embodiments, the subject with treatment resistant depression has been diagnosed with bipolar disorder and has failed to respond to 1 treatment attempt. In some embodiments, the subject with treatment resistant depression has been diagnosed with bipolar disorder and has failed to respond to 2 treatment attempts.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the disease or disorder is an anxiety disorder. As used herein, the term “anxiety disorder” refers to a state of apprehension, uncertainty, and/or fear resulting from the anticipation of an event and/or situation. Anxiety disorders cause physiological and psychological signs or symptoms. Non-limiting examples of physiological symptoms include muscle tension, heart palpitations, sweating, dizziness, shortness of breath, tachycardia, tremor, fatigue, worry, irritability, and disturbed sleep. Non-limiting examples of psychological symptoms include fear of dying, fear of embarrassment or humiliation, fear of an event occurring, etc. Anxiety disorders also impair a subject's cognition, information processing, stress levels, and immune response. In some embodiments, the methods disclosed herein treat chronic anxiety disorders. As used herein, a “chronic” anxiety disorder is recurring. Examples of anxiety disorders include, but are not limited to, generalized anxiety disorder (GAD), social anxiety disorder, panic disorder, panic attack, a phobia-related disorder (e.g., phobias related to flying, heights, specific animals such as spiders/dogs/snakes, receiving injections, blood, etc., agoraphobia), separation anxiety disorder, selective mutism, anxiety due to a medical condition, post-traumatic stress disorder (PTSD), obsessive-compulsive disorder (OCD), substance-induced anxiety disorder, etc.

In some embodiments, the subject in need thereof develops an anxiety disorder after experiencing the effects of a disease. The effects of a disease include diagnosis of an individual with said disease, diagnosis of an individual's loved ones with said disease, social isolation due to said disease, quarantine from said disease, or social distancing as a result of said disease. In some embodiments, an individual is quarantined to prevent the spread of the disease. In some embodiments, the disease is COVID-19, SARS, or MERS. In some embodiments, a subject develops an anxiety disorder after job loss, loss of housing, or fear of not finding employment.

In some embodiments, the disease or disorder is generalized anxiety disorder (GAD). Generalized anxiety disorder is characterized by excessive anxiety and worry, fatigue, restlessness, increased muscle aches or soreness, impaired concentration, irritability, and/or difficulty sleeping. In some embodiments, a subject with generalized anxiety disorder does not have associated panic attacks. In some embodiments, the methods herein are provided to a subject with generalized anxiety disorder also having symptoms of depression. In some embodiments, after treating the symptom(s) is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is social anxiety disorder. As used herein, “social anxiety disorder” is a marked fear or anxiety about one or more social situations in which the individual is exposed to possible scrutiny by others. Non-limiting examples of situations which induce social anxiety include social interactions (e.g., having a conversation, meeting unfamiliar people), being observed (e.g., eating or drinking), and performing in front of others (e.g., giving a speech). In some embodiments, the social anxiety disorder is restricted to speaking or performing in public. In some embodiments, treating according to the methods of the disclosure reduces or ameliorates a symptom of social anxiety disorder. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, body dysmorphic disorder, hoarding disorder, dermatillomania, trichotillomania, excoriation, substance-induced obsessive compulsive and related disorder, or an obsessive-compulsive disorder due to another medical condition, etc., or a combination thereof. In some embodiments, the disease or disorder is obsessive-compulsive disorder (OCD).

In some embodiments, at least one sign or symptom of an anxiety disorder is improved following treatment disclosed herein. In some embodiments, a sign or symptom of an anxiety disorder is measured according to a diary assessment, an assessment by a clinician or caregiver, or a clinical scale. In some embodiments, treatment causes a demonstrated improvement in one or more of the following: State-Trait Anxiety Inventory (STAI), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-1-5), Structured Interview for PTSD (SI—PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Dis-order 7-Item Scale, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI—R), Quality of Life (QoL), The Clinical Global Improvement (CGI) scale, The Yale-Brown Obsessive-Compulsive Scale (Y—BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y—BOCS—SR), The Obsessive-Compulsive Inventory-Re—vised (OCI—R), and the Dimensional Obsessive-Compulsive Scale (DOCS), or a combination thereof. In some embodiments, treating according to the methods of the disclosure results in an improvement in an anxiety disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is attention deficit disorder (ADD). ADD is most commonly diagnosed in children under the age of 16 who have 6 or more symptoms of inattention (5 or more for older teenagers) for at least 6 consecutive months, but no signs of hyperactivity/impulsivity. The symptoms of inattention include, but are not limited to, trouble paying attention, avoids long mental tasks such as homework, trouble staying on task, disorganized or forgetful, doesn't appear to listen when spoken to, doesn't pay close attention to details. Loses things often, makes careless mistakes, and struggles to follow through with instructions. In some embodiments, the disease or disorder is attention deficit hyperactivity disorder (ADHD). ADHD is marked by an ongoing pattern of inattention and/or hyperactivity-impulsivity. Hyperactivity-impulsivity symptoms may often include, but are not limited to, fidgeting or squirming while seated, leaving their seats in situations where staying seated is expected, running, dashing, or climbing around at inappropriate times, being unable to engage in hobbies quietly, being constantly in motion, talking excessively, answering questions before they are fully asked, having difficulty waiting for one's turn, and interrupting or intruding on others during conversations or activities.

In some embodiments, the disease or disorder is a headache disorder. As used herein, the term “headache disorder” refers to a disorder characterized by recurrent headaches. Headache disorders include migraine, tension-type headache, cluster headache, and chronic daily headache syndrome.

In some embodiments, a method of treating cluster headaches in a subject in need thereof is disclosed herein. In some embodiments, at least one sign or symptom of cluster headache is improved following treatment. In some embodiments, the sign or symptom of cluster headache is measured according to a diary assessment, a physical or psychological assessment by clinician, an imaging test, or a neurological examination. Cluster headache is a primary headache disorder and belongs to the trigeminal autonomic cephalalgias. The definition of cluster headaches is a unilateral headache with at least one autonomic symptom ipsilateral to the headache. Attacks are characterized by severe unilateral pain predominantly in the first division of the trigeminal nerve—the fifth cranial nerve whose primary function is to provide sensory and motor innervation to the face. Attacks are also associated with prominent unilateral cranial autonomic symptoms and subjects often experience agitation and restlessness during attacks. In some embodiments, a subject with cluster headaches also experiences nausea and/or vomiting. In some embodiments, a subject with cluster headaches experiences unilateral pain, excessive tearing, facial flushing, a droopy eyelid, a constricted pupil, eye redness, swelling under or around one or both eyes, sensitivity to light, nausea, agitation, and restlessness.

In some embodiments, a method of treating migraines in a subject in need thereof is disclosed herein. A migraine is a moderate to severe headache that affects one half or both sides of the head, is pulsating in nature, and last from 2 to 72 hours. Symptoms of migraine include headache, nausea, sensitivity to light, sensitivity to sound, sensitivity to smell, dizziness, difficulty speaking, vertigo, vomiting, seizure, distorted vision, fatigue, or loss of appetite. Some subjects also experience a prodromal phase, occurring hours or days before the headache, and/or a postdromal phase following headache resolution. Prodromal and postdromal symptoms include hyperactivity, hypoactivity, depression, cravings for particular foods, repetitive yawning, fatigue and neck stiffness and/or pain. In some embodiments, the migraine is a migraine without aura, a migraine with aura, a chronic migraine, an abdominal migraine, a basilar migraine, a menstrual migraine, an ophthalmoplegic migraine, an ocular migraine, an ophthalmic migraine, or a hemiplegic migraine. In some embodiments, the migraine is a migraine without aura. A migraine without aura involves a migraine headache that is not accompanied by a headache. In some embodiments, the migraine is a migraine with aura. A migraine with aura is primarily characterized by the transient focal neurological symptoms that usually precede or sometimes accompany the headache. Less commonly, an aura can occur without a headache, or with a non-migraine headache. In some embodiments, the migraine is a hemiplegic migraine. A hemiplegic migraine is a migraine with aura and accompanying motor weakness. In some embodiments, the hemiplegic migraine is a familial hemiplegic migraine or a sporadic hemiplegic migraine. In some embodiments, the migraine is a basilar migraine. A subject with a basilar migraine has a migraine headache and an aura accompanied by difficulty speaking, world spinning, ringing in ears, or a number of other brainstem-related symptoms, not including motor weakness. In some embodiments, the migraine is a menstrual migraine. A menstrual migraine occurs just before and during menstruation. In some embodiments, the subject has an abdominal migraine. Abdominal migraines are often experienced by children. Abdominal migraines are not headaches, but instead stomach aches. In some embodiments, a subject with abdominal migraines develops migraine headaches. In some embodiments, the subject has an ophthalmic migraine also called an “ocular migraine.” Subjects with ocular migraines experience vision or blindness in one eye for a short time with or after a migraine headache. In some embodiments, a subject has an ophthalmoplegic migraine. Ophthalmoplegic migraines are recurrent attacks of migraine headaches associated with paresis of one or more ocular cranial nerves. In some embodiments, the subject in need of treatment experiences chronic migraines. As defined herein, a subject with chronic migraines has more than fifteen headache days per month. In some embodiments, the subject in need of treatment experiences episodic migraines. As defined herein, a subject with episodic migraines has less than fifteen headache days per month.

In some embodiments, a method of treating chronic daily headache syndrome (CDHS) in a subject in need thereof is disclosed herein. A subject with CDHS has a headache for more than four hours on more than 15 days per month. Some subjects experience these headaches for a period of six months or longer. CHDS affects 4% of the general population. Chronic migraine, chronic tension-type headaches, new daily persistent headache, and medication overuse headaches account for the vast majority of chronic daily headaches.

In some embodiments, after treating according to the methods of the disclosure, the frequency of headaches and/or related symptoms decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a headache attack decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, at least one sign or symptom of headache disorder is improved following administration of a compound disclosed herein. In some embodiments, a sign or symptom of a headache disorder is measured according to a diary assessment, an assessment by a clinician or caregiver, or a clinical scale. In some embodiments, treatment of the present disclosure causes a demonstrated improvement in one or more of the following: the Visual Analog Scale, Numeric Rating Scale, the Short Form Health Survey, Profile of Mood States, the Pittsburgh Sleep Quality Index, the Major Depression Inventory, the Perceived Stress Scale, the 5-Level EuroQoL-5D, the Headache Impact Test; the ID-migraine; the 3-item screener; the Minnesota Multiphasic Personality Inventory; the Hospital Anxiety and Depression Scale (HADS), the 50 Beck Depression Inventory (BDI; both the original BD151 and the second edition, BDI-1152), the 9-item Patient Health Questionnaire (PHQ-9), the Migraine Disability Assessment Questionnaire (MI-DAS), the Migraine-Specific Quality of Life Questionnaire version 2.1 (MSQ v2.1), the European Quality of Life-5 Dimensions (EQ-5D), the Short-form 36 (SF-36), or a combination thereof. In some embodiments, treating according to the methods of the disclosure results in an improvement in a headache disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art. In some embodiments, the sign or symptom of the headache disorder is measured according to a diary assessment, a physical or psychological assessment by clinician, an imaging test, an electroencephalogram, a blood test, a neurological examination, or combination thereof. In some embodiments, the blood test evaluates blood chemistry and/or vitamins.

In some embodiments, the disease or disorder is a substance use disorder. Substance addictions which can be treated using the methods herein include addictions to addictive substances/agents such as recreational drugs and addictive medications. Examples of addictive substances/agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine. In some embodiments, the disease or disorder is alcohol use disorder (AUD). In some embodiments, the disease or disorder is nicotine use (e.g., smoking) disorder, and the therapy is used for e.g., smoking cessation. In some embodiments, the disease or disorder is opioid use disorder. In some embodiments, the disease or disorder is amphetamine use disorder. In some embodiments, the disease or disorder is cocaine use disorder.

In some embodiments, the disease or disorder is an eating disorder. As used herein, the term “eating disorder” refers to any of a range of psychological disorders characterized by abnormal or disturbed eating habits. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof. In some embodiments, the eating disorder is pica, anorexia nervosa, bulimia nervosa, rumination disorder, avoidant/restrictive food intake disorder, binge-eating disorder, or combinations thereof. In some embodiments, the methods disclosed herein treat chronic eating disorders. As used herein, a “chronic” eating disorder is recurring. In some embodiments, at least one sign or symptom of an eating disorder is improved following administration of a compound disclosed herein. In some embodiments, a sign or symptom of an eating disorder is measured according to a diary assessment, an assessment by a clinician or caregiver, or a clinical scale. Non-limiting examples of clinical scales, diary assessments, and assessments by a clinician or caregiver include: the Mini International Neuropsychiatric Interview (MINI), the McLean Screening Instrument for Borderline Personality Disorder (MSI-BPD), the Eating Disorder Examination (EDE), the Eating Disorder Questionnaire (EDE-Q), the Eating Disorder Examination Questionnaire Short Form (EDE-QS), the Physical Appearance State and Trait Anxiety Scale-State and Trait version (PASTAS), Spielberger State-Trait Anxiety Inventory (STAI), Eating Disorder Readiness Ruler (ED-RR), Visual Analogue Rating Scales (VAS), the Montgomery-Asberg Depression Rating Scale (MADRS), Yale-Brown Cornell Eating Disorder Scale (YBC-EDS), Yale-Brown Cornell Eating Disorder Scale Self Report (YBC-EDS-SRQ), the Body Image State Scale (BISS), Clinical impairment assessment (CIA) questionnaire, the Eating Disorder Inventory (EDI) (e.g. version 3: EDI-3), the Five Dimension Altered States of Consciousness Questionnaire (5D-ASC), the Columbia-Suicide Severity Rating Scale (C—SSRS), the Life Changes Inventory (LCI), and combinations thereof. In some embodiments, treating according to the methods of the disclosure results in an improvement in an eating disorder compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is multiple sclerosis (MS). MS is a chronic, inflammatory disease of unknown etiology that involves an immune-mediated attack on the central nervous system. Myelin and the oligodendrocytes that form myelin appear to be the primary targets of the inflammatory attack, although the axons themselves are also damaged. MS disease activity can be monitored by cranial scans, including magnetic resonance imaging (MRI) of the brain, accumulation of disability, as well as rate and severity of relapses. The diagnosis of clinically definite MS as determined by the Poser criteria requires at least two neurological events suggesting demyelination in the CNS separated in time and in location. Various MS disease stages and/or types are described in Multiple Sclerosis Therapeutics. London: Martin Dunitz; 1999:349-370. Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination. In some embodiments, the multiple sclerosis is relapsing multiple sclerosis. In some embodiments, the relapsing multiple sclerosis is relapsing-remitting multiple sclerosis. In some embodiments, the methods herein reduce a symptom of multiple sclerosis in the subject. In some embodiments, the symptom is a MRI-monitored multiple sclerosis disease activity, relapse rate, accumulation of physical disability, frequency of relapses, decreased tune to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work. In some embodiments, the methods herein decrease or inhibit reduction of brain volume. In some embodiments, brain volume is measured by percent brain volume change (PBVC). In some embodiments, the methods herein increase time to confirmed disease progression. In some embodiments, time to confirmed disease progression is increased by at least 10%, at least 20%, at least 30%, at least 40%, at least 50%, for example at least 20-60%. In some embodiments, the methods herein decrease abnormalities observed in whole Brain MTR histogram. In some embodiments, the accumulation of physical disability is measured by Kurtzke Expanded Disability Status Scale (EDSS) score. In some embodiments, the accumulation of physical disability is assessed by the time to confirmed disease progression as measured by Kurtzke Expanded Disability Status Scale (EDSS) score.

In some embodiments, the disease or disorder is a disease or disorder characterized by, or otherwise associated with, neuroinflammation. In some embodiments, the disease or disorder is a disease or disorder characterized by, or otherwise associated with, decreasing neuroplasticity. Treatment herein may provide cognitive benefits to subject's suffering from neurological and neurodegenerative diseases such as Alzheimer's disease and other dementia subtypes, Parkinson's disease, amyotrophc lateral sclerosis (ALS), and others where neuroinflammation is a hallmark of disease pathophysiology and progression. For example, emerging psychedelic research/clinical evidence indicates that psychedelics may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics are thought to stimulate neurogenesis, provoke neuroplastic changes, and to reduce neuroinflammation. Thus, in some embodiments, the methods of the present disclosure are used for the treatment of neurological and neurodegenerative disorders such as Alzheimer's disease, dementia subtypes, Parkinson's disease, and amyotrophc lateral sclerosis (ALS), where neuroinflammation is associated with disease pathogenesis. In some embodiments, the methods of the present disclosure are used for the treatment of Alzheimer's disease. In some embodiments, the methods of the present disclosure are used for the treatment of dementia. In some embodiments, the methods of the present disclosure are used for the treatment of Parkinson's disease. In some embodiments, the methods of the present disclosure are used for the treatment of amyotrophc lateral sclerosis (ALS). As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). In some embodiments, treating according to the methods of the disclosure results in an improvement in cognition in subject's suffering from a neurological or neurodegenerative disease compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of a diary assessments, assessments by a clinical or caregiver, or clinical scales, described herein or known in the art.

Further, many of the behavioral issues associated with chronic and/or life-threatening illnesses, including neurodegenerative disorders such as Alzheimer's disease, may benefit from treatments disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease, as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. Methods described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder, including neurological and neurodegenerative diseases. In some embodiments, the methods provided herein reduce at least one sign or symptom of a neurological and/or neurodegenerative disease. In some embodiments, the methods provided herein reduce at least one sign or symptom of a neurological and/or neurodegenerative disease (e.g., depression, anxiety, and/or stress) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment, e.g., according to any one of the diary assessments, assessments by a clinical or caregiver, or clinical scales, described herein or known in the art.

In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is amyotrophc lateral sclerosis (ALS). In some embodiments, the methods herein are used for the treatment of depression, anxiety, and/or stress associated with amyotrophc lateral sclerosis (ALS). In some embodiments, the disease or disorder is cancer related depression and anxiety. In some embodiments, blood concentrations of active ingredient (e.g., 5-HT_{2A} receptor agonist) are kept below the psychedelic threshold.

In some embodiments, the methods disclosed herein are used for treatment of brain injury, including traumatic brain injury (TBI). TBI is an injury to the brain caused by an external force, and can be classified based on severity, ranging from mild traumatic brain injury (mTBI/concussion) to severe traumatic brain injury. TBI can also be categorized by mechanism, as either a closed or penetrating head injury, or other features such as whether it is occurring in a specific location or over a widespread area. TBI can result in physical, cognitive, social, emotional and behavioral symptoms, which may be treated herein. Some of the imaging techniques used for diagnosis and recovery markers include computed tomography (CT) and magnetic resonance imaging (MRIs).

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs. Subjects with autism spectrum disorder may present with various signs and symptoms, including, but not limited to, a preference for non-social stimuli, aberrant non-verbal social behaviors, decreased attention to social stimuli, irritability, anxiety (e.g., generalized anxiety and social anxiety in particular), and depression. In some embodiments, the autism spectrum disorder comprises a medical diagnosis based on the criteria and classification from Diagnostic and Statistical Manual of Mental Disorders, 5th Ed (DSM-5). Current evidence supports the use of psychedelics for ameliorating behavior atypicalities of autism spectrum disorder, including reduced social behavior, anxiety, and depression (see Markopoulos A, Inserra A, De Gregorio D, Gobbi G. Evaluating the Potential Use of Serotonergic Psychedelics in Autism Spectrum Disorder. Front Pharmacol. 2022; 12:749068). The signs and symptoms of autism spectrum disorder may be treated with the methods herein.

In some embodiments, the disease or disorder is a genetic condition that causes learning disabilities and cognitive impairment. An example of such a genetic condition is fragile X syndrome, caused by changes in the gene Fragile X Messenger Ribonucleoprotein 1 (FMR1), which can cause mild to moderate intellectual disabilities in most males and about one-third of affected females. Fragile X syndrome and autism spectrum disorder are closely associated because the FMR1 gene is a leading genetic cause of autism spectrum disorder (see Markopoulos A, Inserra A, De Gregorio D, Gobbi G. Evaluating the Potential Use of Serotonergic Psychedelics in Autism Spectrum Disorder. Front Pharmacol. 2022; 12:749068). Subjects with fragile X syndrome may display anxiety, hyperactive behavior (e.g., fidgeting and impulsive actions), attention deficit disorder, mood and aggression abnormalities, poor recognition memory, and/or features of autism spectrum disorder, and these signs and symptoms may be treated with the methods herein. Clinical trials with psychedelics for the treatment of fragile X syndrome and autism spectrum disorder are currently ongoing (ClinicalTrials.gov, number NCT04869930).

In some embodiments, the disease or disorder is mental distress, e.g., mental distress in frontline healthcare workers.

In some embodiments, the compounds and compositions disclosed herein are used for treatment of tic disorders, including Tourette's Syndrome, which is also variously referred to as Tourette Syndrome, Tourette's Disorder, Gilles de la Tourette syndrome (GTS), or simply Tourette's or TS. The tic disorder may also be a pediatric autoimmune disorder associated with streptococcal infection (PANDAS), a transient tic disorder, a chronic tic disorder, or a tic disorder not otherwise specified (NOS). Tic disorders are defined in the Diagnostic and Statistical Manual of Mental Disorders (DSM) based on type (motor or phonic) and duration of tics (sudden, rapid, nonrhythmic movements), or similarly by the World Health Organization (ICD-10 codes). Tics are involuntary or semi-voluntary, sudden, brief, intermittent, repetitive movements (motor) or sounds (phonic) that are classified as simple or complex. Simple tics, for example, eye blinking or facial grimacing, are relatively easy to camouflage and may go largely unnoticed. Complex tics, such as body contortions, self-injurious behavior, obscene gestures, or shouting of socially inappropriate word or phrases, can appear to be purposeful actions and are particularly distressing. Transient tic disorders are generally characterized by multiple motor and/or phonic tics that occur for at least four weeks but less than 12 months. Chronic tic disorders are generally characterized by either single or multiple motor or phonic tics, but not both, which are present for more than a year. Tourette's Syndrome is diagnosed when both motor and phonic tics are present (although not necessarily concurrently) for more than one year. Thus, Tourette's syndrome (TS) is a chronic neuropsychiatric disorder characterized by the presence of fluctuating motor and phonic tics. The typical age of onset is between five and seven years. Affected children may become the target of teasing by peers, which in turn can result in low self-esteem, social isolation, poor school performance, depression and anxiety. In addition to causing social embarrassment, sudden, forceful tics can be painful, and violent head and neck tics have been reported to cause secondary neurologic deficits, such as compressive cervical myelopathy. Tourette's Syndrome patients are also at increased risk for obsessive-compulsive disorder (OCD), depression, and attention-deficit-hyperactivity disorder (ADHD). Tic disorder NOS is diagnosed when tics are present but do not meet the criteria for any specific tic disorder. The methods of the present disclosure can also be used for the treatment of tics induced as a side effect of a medication; tics associated with autism; and Tourettism (the presence of Tourette-like symptoms in the absence of Tourette's Syndrome (e.g., as a result of another disease or condition, such as a sporadic, genetic, or neurodegenerative disorder)).

Efficacy of the treatment disclosed herein may in some cases be assessed through clinical interviews where patients answer a series of questionnaires, which allows for quantification of different aspects of psychedelic-induced subjective effects. These assessments can include, but are not limited to, Mystical Experience Questionnaire-30 Item (MEQ-30) (see Maclean, K. A., Leoutsakos, J.-M. S., Johnson, M. W. & Griffiths, R. R. Factor Analysis of the Mystical Experience Questionnaire: A Study of Experiences Occasioned by the Hallucinogen Psilocybin. J Sci Study Relig 51, 721-737 (2012)), 5-Dimensional Altered States of Consciousness Rating Scale (5D-ASC) (see Dittrich, A. The Standardized Psychometric Assessment of Altered States of Consciousness (ASCs) in Humans. Pharmacopsychiatry 31, 80-84 (1998)), and the Hallucinogen Rating Scale (HRS) (see Strassman, R. J., Qualls, C. R., Uhlenhuth, E. H. & Kellner, R. Dose-Response Study of N,N-Dimethyltryptamine in Humans: II. Subjective Effects and Preliminary Results of a New Rating Scale. Archives of General Psychiatry 51, 98-108 (1994)).

The methods of delivering a psychedelic drug to the CNS (systemic drug delivery) via inhalation, such as through a nebulizer or other device as described herein (including, for example, using a heated helium-oxygen mixture), can lead to advantageous improvements in multiple PK parameters as compared to other delivery routes such as oral and intravenous delivery. These effects from inhalational delivery may be particularly pronounced when the psychedelic drug is dimethyltryptamine (DMT) or a deuterated analog thereof. In particular, a psychedelic drug can cross the blood brain barrier more efficiently and be delivered to the brain with the inhalation administration disclosed herein. As compared to oral delivery, the method of delivering a psychedelic drug to the CNS via inhalation, such as with a nebulizer or other device as described herein, optionally with a heated heliox mixture, can increase bioavailability by at least 25% as compared to oral delivery of the psychedelic drug. In some embodiments, the method of delivering a psychedelic drug to the CNS via inhalation, such as with a nebulizer or other device as described herein, can increase bioavailability by about 10%, 25%, 30%, 35%, 40%, 50%, 55%, 60%, 65%, 70%, 80%, 85%, 90%, 95%, 99%, 99.9%, 100%, 200%, 300%, 400%, 500%, 600%, 700%, 800%, 900%, 1,000%, 1,500%, 2,000%, or more, or any range therebetween, as compared to oral delivery of the psychedelic drug. In some embodiments, the method of delivering a psychedelic drug to the CNS via inhalation, such as with a nebulizer or other device as described herein, can reduce Tₘₐₓ by at least 30%, 40%, 50%, 55%, 60%, 65%, 70%, 75%, 80%, 85%, 90%, 95%, 99%, 99.9%, or more, or any range therebetween, as compared to oral delivery. In some embodiments, the method of delivering a psychedelic drug to the CNS via inhalation, such as with a nebulizer or other device as described herein, can increase Cₘₐₓ by about 10%, 25%, 30%, 35%, 40%, 50%, 55%, 60%, 65%, 70%, 80%, 85%, 90%, 95%, 99%, 99.9%, 100%, 200%, 300%, 400%, 500%, 600%, 700%, 800%, 900%, 1,000%, 1,500%, 2,000%, 2,200%, 2,400%, 2,600%, 2,800%, 3,000%, or more, or any range therebetween, as compared to oral delivery of the psychedelic drug. In some embodiments, the method of delivering a psychedelic drug to the CNS via inhalation, such as with a nebulizer or other device as described herein, can increase the duration of exposure (MRTₗₐₛₜ) by about 10%, 25%, 30%, 35%, 40%, 50%, 55%, 60%, 65%, 70%, 80%, 85%, 90%, 95%, 99%, 99.9%, 100%, 200%, 300%, 400%, 500%, 600%, 700%, 800%, 900%, 1,000%, or more, or any range therebetween, as compared to intravenous (bolus) delivery of the psychedelic drug. Furthermore, a method of delivering a psychedelic drug to the CNS via inhalation via a nebulizer or other device as described herein, can allow clinical protocols enabling dose titration and more controlled exposure. Controlled exposure enables adjusting the patient experience and providing overall improved therapeutic outcomes. With the smart technology enabled devices for inhalation delivery noted above, the dose titration and controlled delivery can be performed remotely by the healthcare worker, enabling the patient to be in the comfort of their own home, improving the patient's experience and outcome.

Deuteration has also been found to enhance inhalational delivery of psychedelic drugs compared to their non-deuterium enriched counterparts (i.e., deuterium levels at the natural abundance found in hydrogen) administered by the same method. In some embodiments, the method of delivering a deuterated psychedelic drug to the CNS via inhalation, such as with a nebulizer or other device as described herein, can increase bioavailability by about 10%, 15%, 20%, 25%, 30%, 35%, 40%, 45%, 50%, or more, or any range therebetween, as compared to delivery of the corresponding non-deuterium enriched psychedelic drug administered by the same method. In some embodiments, the method of delivering a deuterated psychedelic drug to the CNS via inhalation, such as with a nebulizer or other device as described herein, can increase the duration of exposure (MRTₗₐₛₜ) by about 5%, 10%, 15%, 20%, 25%, 30%, 35%, or more, or any range therebetween, as compared to delivery of the corresponding non-deuterium enriched psychedelic drug administered by the same method.

In some embodiments, a system is provided for administering psychedelic drugs (including salts thereof) that includes a container comprising a solution of a psychedelic drug formulation and a nebulizer physically coupled or co-packaged with the container and adapted to produce an aerosol, such as a mist, of the solution having a particle size from about 0.1 microns to about 10 microns (e.g., about 10, 5, 4, 3, 2, 1, 0.1 or less microns).

A patient or subject can be any mammal including, for example, a human. A patient or subject can have a condition to be treated or can be susceptible to a condition to be treated.

In some embodiments, the treatment with the psychedelic drug of present disclosure may be used as a standalone therapy. In some embodiments, the treatment with the psychedelic drug of present disclosure may be used as an adjuvant/combination therapy with other treatment modalities and/or agents. For example, treatment with the psychedelic drug may be performed in conjunction with psychotherapy, psycho-social therapy (e.g., cognitive behavioral therapy), and/or treatment with other agents such as an anxiolytic or antidepressant (conventional). Examples of anxiolytics/antidepressants include, but are not limited to, barbiturates; benzodiazepines such as alprazolam, bromazepam, chlordiazepoxide, clonazepam, diazepam, lorazepam, oxazepam, temazepam, and triazolam; selective serotonin reuptake inhibitors (SSRIs) such as citalopram, escitalopram, fluoxetine, fluvoxamine, paroxetine, and sertraline; serotonin-norepinephrine reuptake inhibitors (SNRIs) such as venlafaxine, duloxetine, atomoxetine, desvenlafaxine, levomilnacipran, milnacipran, sibutramine, and tramadol; serotonin modulator and stimulators (SMSs) such as vortioxetine and vilazodone; serotonin antagonist and reuptake inhibitors (SARIs) such as trazodone and nefazodone; norepinephrine reuptake inhibitors (NRIs or NERIs) such as atomoxetine, reboxetine, and viloxazine; norepinephrine-dopamine reuptake inhibitors such as bupropion; tricyclic antidepressants (TCAs) such as imipramine, doxepin, amitriptyline, nortriptyline and desipramine; tetracyclic antidepressants such as mirtazapine; monoamine oxidase inhibitors (MAOIs) such as phenelzine, isocarboxazid, tranylcypromine and pyrazidol; sympatholytics such as propranolol, oxprenolol, metoprolol, prazosin, clonidine, and guanfacine; and others such as buspirone, pregabalin, and hydroxyzine.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### I. DMT and DMT-d₁₀: Pharmacokinetic (PK) Study by Intravenous (Bolus), Oral Gavage, and Inhalation Administration to Male Rats

The pharmacokinetics and bioavailability of N,N-dimethyltryptamine (DMT) and 2-(1H-indol-3-yl)-N,N-bis(methyl-d₃)ethan-1-amine-1,1,2,2-d₄ (DMT-d₁₀) were investigated in rats following intravenous (bolus), oral gavage (OG), and inhalation after co-dose administration. The experimental conditions and results are presented below.

Animals. Twenty-nine male Sprague Dawley rats aged 7-10 weeks and weighing between 210-290 g at dosing were used. Animals were supplied by a recognized supplier of laboratory animals.

Housing. The in-life experimental procedures were subject to the provisions of the United Kingdom Animals (Scientific Procedures) Act 1986 Amendment Regulations 2012 (the Act). The number of animals used were the minimum that is consistent with scientific integrity and regulatory acceptability, consideration having been given to the welfare of individual animals in terms of the number and extent of procedures to be carried out on each animal.

Animals were uniquely identified by tattoo or by microchip. During the pre-trial holding periods, the animals were group housed in caging appropriate to the species. Rats were housed 3 per cage with access to food (Teklad 2014C, pelleted diet) and quality tap water ad libitum. Animals were checked regularly throughout the duration of the study. Any clinical signs were closely monitored and recorded. There was limited access to the animal facility to minimize external exposure to biological and chemical agents. Air supply was filtered and not re-circulated. Temperature and humidity were within the ranges of 20-24° C. and 40-70%, respectively. Lighting was 12 hours light; 12 hours dark.

Test Items. DMT (fumarate salt) and DMT-d₁₀. Both test items were formulated as solutions in vehicle. The vehicle used was citrate (0.1 M) buffer, pH 6.0. To prepare the vehicle, citric acid monohydrate+trisodium citrate dihydrate were weighed into a suitable sized container, dissolved in ca. 90% of final volume of water for injection (WFI), and magnetically stirred to mix. The pH was checked and adjusted to 6.0±0.1 using NaOH or HCl, and the strengths and volumes were recorded. The final volume was made with WFI, and magnetically stirred to mix. The vehicle was then filtered through a 0.22 μm PVDF filter. Some vehicle was dispensed into the appropriate containers for the control group prior to starting the test formulations, with sampling performed at this point, if required. The test item was acclimated to room temperature before use and weighed in the required amount (weighing may be performed in advance). ca. 50% of the final volume of vehicle was added to the test item to obtain a solution, washing the container containing both test item weighing's. An initial mix, with crushing any large particles, may be made by hand using a spatula. If required, the mixture was transferred to a larger container. Dissolution and mixing were performed using a magnetic stirrer, and the start and finish times were recorded. Sonication was used to aide in dissolution if needed. The pH was checked and adjusted to 6.0±0.1 with NaOH or HCl. Strengths and volumes were recorded. The test item solutions were transferred to a measuring cylinder and made up to final volume with remaining vehicle and stirred for a minimum of 20 minutes using a magnetic stirrer. The final pH was checked and recorded (adjusted if necessary), as was the osmolarity. Sampling was performed at this point, if required, whilst magnetically stirring. The solutions were transferred to final containers, via syringe, whilst magnetically stirring.

The following correction factors were used:
i. 1.62 for DMT (fumarate)
ii. 1.05 for DMT-d₁₀(free base)

### Nominal Co-Administration Dose Levels.

IV and oral: DMT+DMT-d₁₀: 1.62 mg/mL+1.05 mg/mL
Inhalation: DMT+DMT-d₁₀: 81.0 mg/mL+83.5 mg/mL

Experimental Design. This was a single use study with 4 treatment groups as outlined in Table 1.

TABLE 1
Treatment Groups - Co-Administration of DMT and DMT-d₁₀

| Group | Number | | | Dose | Animal |
|---|---|---|---|---|---|
| # | and Sex | Dose route | Treatments | (mg/kg)^{b)} | Numbers |
| 1^{a)} | 3M | Intravenous | DMT + DMT-d₁₀ | 1 + 1 | 1-3 |
| | | (bolus) | | | |
| 2 | 3M | Oral gavage | DMT + DMT-d₁₀ | 10 + 10 | 4-6 |
| 4 | 12M | Intravenous | DMT + DMT-d₁₀ | 1 + 1 | 14-25 |
| | | (bolus) | | | |
| 6 | 3M | Inhalation | DMT + DMT-d₁₀ | 14.3 + 15.5 | 28-30 |

^{a)}After the IV administration to Group 1, there were blood sampling technical difficulties that resulted in an inadequate number of samples to determine PK parameters. For this reason, PK parameters for Group 1 are not reported.
^{b)}Co-administration of DMT and DMT-d10; free base dose levels

Animals received a single IV bolus via the lateral tail vein, or an oral dose via flexible gavage tube. For the inhalation dose, animals were placed in an inhalation chamber and received a 20-minute aerosolized exposure. Bodyweights were recorded for each animal prior to dosing.

### Inhalation Procedure.

Pre-study characterization. Before commencement of treatment, the system was characterized at the target aerosol concentrations without animals in order to demonstrate satisfactory particle size, satisfactory operation of the exposure system, and reproducibility of test item concentration.

Test atmosphere generation. A suitable nebulizer (or multiple nebulizers) was used to deliver the inhalation dose. The test substance liquid formulation was added to the reservoir of the nebulizer in bulk or added to the reservoir at a controlled rate by syringe driver. Precise details of the operating conditions were determined to achieve the target droplet aerosol concentrations.

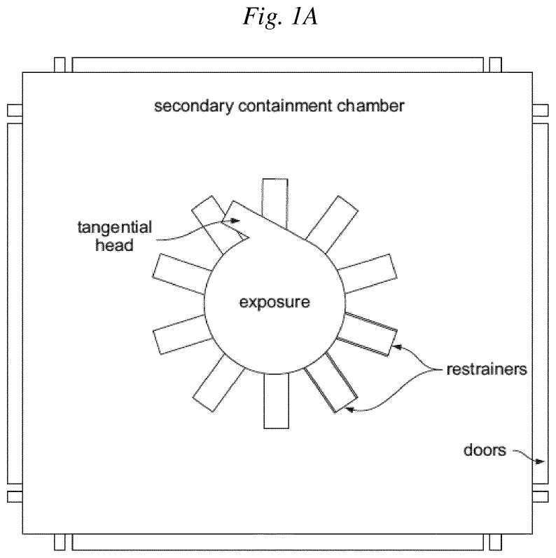

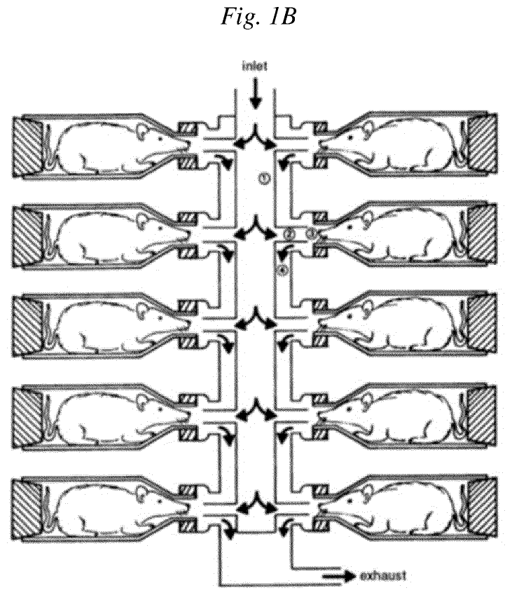

Test atmosphere administration. The inhalation dose was received by snout only exposure. The equipment was a directed flow exposure chamber with modular construction in aluminum alloy comprising a base unit, a variable number of sections each having 8 exposure ports, and a top section incorporating a central aerosol inlet with a tangential air inlet. During exposure, the rats were held in restraining tubes with their snouts protruding from the ends of the tubes into the exposure chambers. Animal exposure ports not in use were closed with blanking plugs. The exposure system was housed in an extract cabinet/secondary containment chamber. The animals on study were acclimated to the method of restraint over at least a 3-day period prior to dosing. The duration of exposure was determined to be 20 minutes. A representation of the directed flow exposure chamber is shown in FIGS. 1A-1B.

Test atmosphere analysis. The inhalation amount of DMT and DMT-d₁₀ were determined from samples collected on filters by gravimetric analysis and the concentration calculated. The particle size of DMT was determined on collections from glass fibre filters. From these data, the mass medium aerodynamic diameter (MMAD) and the geometric standard deviation (ag) of the aerosol was calculated assuming a log-normal distribution of particle size.

The inhalation dose in mg/kg was determined according to equation (1):

$$Dose ( mg / kg ) = \frac{C ( μg / L ) \times RMV ( L / min ) \times D ( min )}{BW ( kg ) \times 1000}$$

where:
   C=Aerosol concentration (μg/L).
   RMV=Respiratory minute volume=0.608×BW^{0.852}
   D=Duration of exposure (20 mins).
   BW=Body weight (kg).

Sampling collection. PK samples (0.3 mL) were collected from the jugular vein by venepuncture into tubes containing K₂EDTA anticoagulant at the following sampling times: Group 1 (IV) and Group 2 (oral) serial plasma collection at 0.083, 0.25, 0.5, 1, 3, 8 and 24 hr postdose; Group 4 (IV) composite plasma and brain collection at 0.083, 0.25, 0.5 and 1 hr postdose; Group 6 (inhalation) serial plasma collection at 0.333, 0.533, 0.833, 1.333, 3.333, 8.333 and 24.333 after start of inhalation.

Plasma samples: Immediately following collection, samples were inverted to ensure mixing with anti-coagulant and placed on wet ice. Plasma was generated by centrifugation (2000 g, 10 min, 4° C.) within 60 min of collection. 90 μL of plasma was transferred into a tube containing 90 μL (1:1 (v/v)) of 200 mM ascorbic acid. Three 50 μL of stabilized plasma samples were aliquoted into polypropylene tubes, frozen on dry ice and stored in −70° C. (±10° C.) until analysis.

Brain samples: After extraction of whole brain from the cranium, brains were rinsed, patted dry, weighed, placed into tubes and frozen on dry ice. Thereafter, they were stored at −70 (±10°) C pending analysis.

Bioanalysis. Plasma and brain homogenates were analyzed for DMT and DMT-d₁₀ using an established LC-MS/MS assay.) Pharmacokinetic parameters were determined from the DMT and DMT-d₁₀ plasma and brain concentration-time profiles using commercially available software (Phoenix® WinNonlin®).

### Results.

After IV dose administration to Group 1, there were sampling technical difficulties that prevented an adequate number of collections to construct reliable concentration-time profiles. For this reason, PK parameters for Group 1 are not presented.

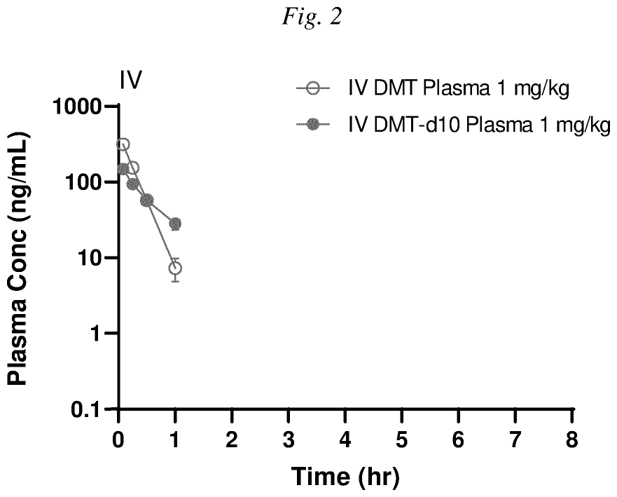

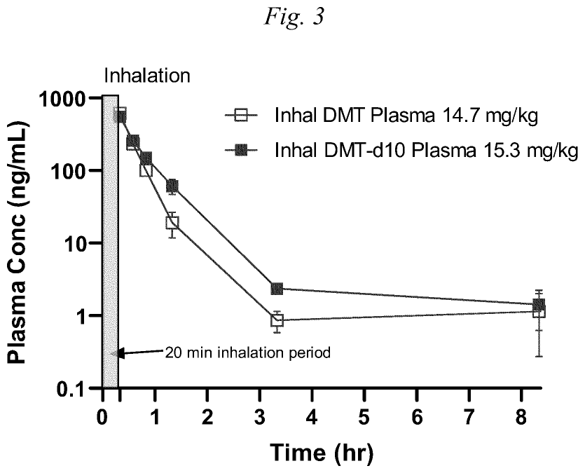

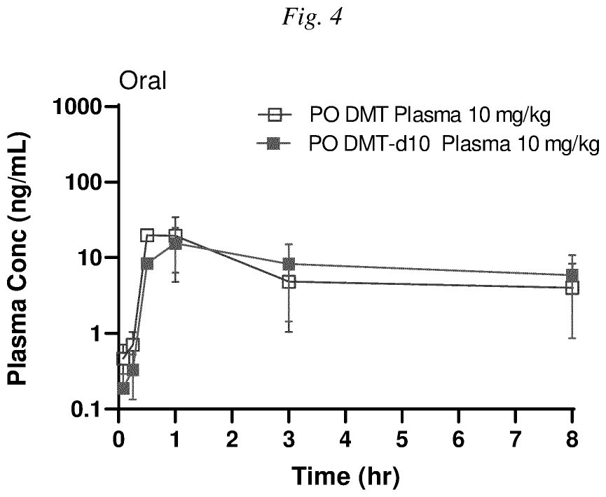

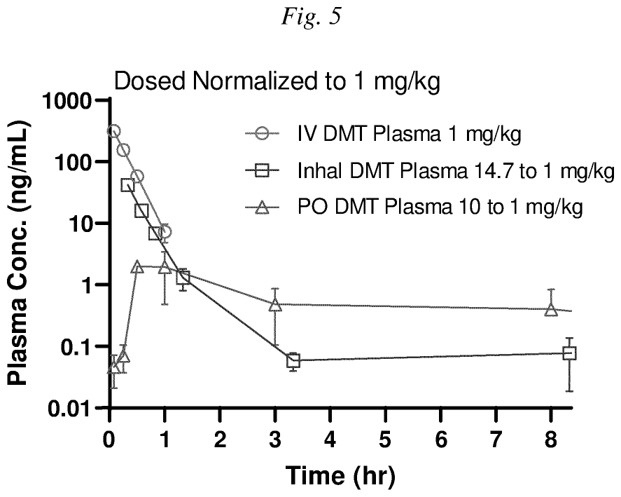

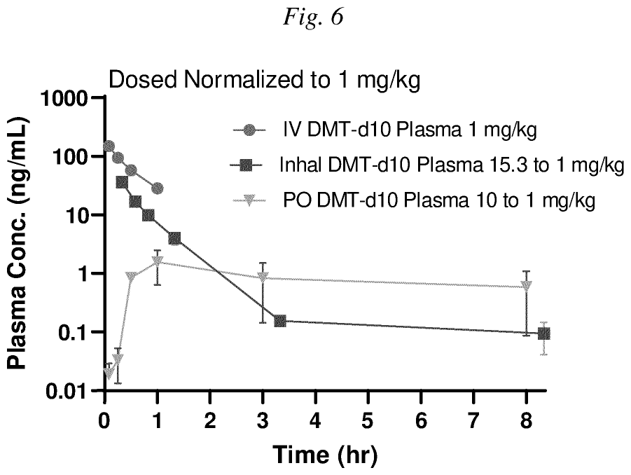

Group 4 replaced and expanded Group 1 with the simultaneous collection of plasma and brain after IV co-administration of DMT and DMT-d₁₀. The mean plasma and brain PK parameters are summarized in Tables 2 and 3, respectively. Group 2 (oral) and Group 6 (inhalation) PK parameters are summarized in Table 2. The PK parameters used to calculate brain to plasma ratios and bioavailability (% F) after oral and inhalation administration of DMT and DMT-d₁₀ are shown in Table 4. The DMT and DMT-d₁₀ plasma concentration-time profiles after IV, inhalation, and oral administration are shown in FIGS. 2, 3, and 4, respectively. FIGS. 5 and 6 represent DMT and DMT-d₁₀ plasma concentration-time profiles normalized to a 1 mg/kg dose, respectively.

Co-administrated doses of DMT and DMT-d₁₀ were 1+1 mg/kg for IV; 10+10 mg/kg for oral and 14.3+15.5 mg/kg for inhalation, respectively. Examination of the plasma concentration-time DMT and DMT-d₁₀ profiles illustrate that plasma exposure after inhalation was as rapid as an IV bolus, with the highest concentrations observed at the first time points taken, 0.333 and 0.083 hr, respectively. Corresponding Cₘₐₓ values of DMT and DMT-d₁₀ were 303 and 148 ng/mL after IV and 598 and 538 ng/mL after inhalation, respectively. In contrast, peak plasma concentrations after oral administration, were achieved 1 hr postdose, with Cₘₐₓ values of 28.0 and 20.8 ng/mL, DMT and DMT-d₁₀, respectively. Matched and dose normalized integrated exposures (AUC₀₋₁/dose) were used to calculate bioavailabilities (% F) of DMT and DMT-d₁₀: 16.3 and 22.6% after inhalation and 1.36 and 1.16% after oral exposure, respectively. The mean residence time (MRT) was approximately 5× greater after inhalation compared to IV administration.

Distribution of DMT and DMT-d₁₀ into brain was high. Brain Cₘₐₓ values were 3430 and 1490 ng/g, respectively, compared to their matched plasma concentrations of 303 and 148 ng/mL, respectively.

Deuteration improved the brain to plasma (B/P) ratio by approximately 30% (12.3 vs 9.5; DMT-d₁₀ vs. DMT, respectively); improved the duration of exposure (MRTₗₐₛₜ) by 24.6 to 54.5% after inhalation and IV; and increased inhalation bioavailability by approximately 40% (22.6% vs. 16.3%, DMT-d₁₀ vs. DMT, respectively), approximately 20× greater than oral bioavailability.

TABLE 2
Plasma pharmacokinetic parameters

| | Dose | Dose Level | | Cₘₐₓ | Tₘₐₓ | AUC₀₋ₜ | AUC_{0-inf} | AUC₀₋₁ | t_{1/2}^{b)} | F ^{c)} |
|---|---|---|---|---|---|---|---|---|---|---|
| Analyte | Group^{a)} | (mg/kg) | Sex | (ng/mL) | (h) | (h*ng/mL) | (h*ng/mL) | (h*ng/mL) | (h) | (%) |
| DMT | 2 (OG) | 10 | Male | 28.0 | 1.00 | 109 | NR | 15.2 | NR | 1.36 |
| DMT | 4 (IV) | 1 | Male | 303 | NA | 112 | 114 | 112 | 0.172 | NA |
| DMT | 6 (Inh) | 14.3 | Male | 598 | 0.333 | 308 | NR | 260 | NR | 16.3 |
| DMT-d₁₀ | 2 (OG) | 10 | Male | 20.8 | 1.00 | 80.2 | NR | 8.78 | NR | 1.16 |
| DMT-d₁₀ | 4 (IV) | 1 | Male | 148 | NA | 75.4 | 92.6 | 75.4 | 0.446 | NA |
| DMT-d₁₀ | 6 (Inh) | 15.5 | Male | 538 | 0.333 | 383 | 384 | 264 | 5.42 | 22.6 |

^{a)}There were sampling technical difficulties with Group 1 that prevented an adequate number of collections to construct reliable concentration-time profiles. For this reason, PK parameters for Group 1 are not presented.
^{b)}Plasma t_{1/2} values calculated: Group 4 (IV), 0.083 to 1.0 hr. Group 6 (Inh), 0.333 to 3.333 hr.
^{c)} Calculated from dose normalized AUC₀₋₁ mean values; Group 4 IV; Group 2 oral; Group 6 inhalation
NA = Not applicable; NR = Not reportable due to an inability to construction a plasma concentration-time profile or characterize the elimination phase.

TABLE 3
Brain pharmacokinetic parameters

| | Dose | Dose Level | | Cₘₐₓ | Tₘₐₓ | AUC₀₋ₜ | AUC_{0-inf} | t_{1/2} ^{a)} |
|---|---|---|---|---|---|---|---|---|
| Analyte | Group | (mg/kg/day) | Sex | (ng/g) | (h) | (h*ng/g) | (h*ng/g) | (h) |
| DMT | 4 (IV) | 1 | Male | 3430 | 0.0833 | 1060 | 1070 | 0.155 |
| DMT-d₁₀ | 4 (IV) | 1 | Male | 1490 | 0.0833 | 931 | NR | NR |

^{a)} Brain t_{1/2} values calculated: Group 4 (IV), 0.25 to 1 hr
NR = Not reportable due to an inability to construction a plasma concentration-time profile or characterize the elimination phase

TABLE 4
Summary pharmacokinetic parameters

| | Dose (mg/kg) | | AUC₀₋₁ (hr*ng/mL) | | AUC₀₋₁/dose | | t_{1/2} (hr)^{a)} | | MRTₗₐₛₜ (hr) | | B/P (AUC₀₋₁) | |
|---|---|---|---|---|---|---|---|---|---|---|---|---|
| Route | DMT | DMT-d₁₀ | DMT | DMT-d₁₀ | DMT | DMT-d₁₀ | DMT | DMT-d₁₀ | DMT | DMT-d₁₀ | DMT | DMT-d₁₀ |
| Intravenous | 1 | 1 | 112 | 75.4 | 112 | 75.4 | 0.172 | 0.446 | 0.211 | 0.326 | 9.5 | 12.3 |
| Inhalation | 14.3 | 15.5 | 260 | 264 | 18.2 | 17.0 | NR | 5.42 | 1.18 | 1.47 | | |
| Oral | 10 | 10 | 15.2 | 8.78 | 1.52 | 0.878 | NR | NR | 2.87 | 3.69 | | |
| % F Inhalation | | | | | 16.3% | 22.6% | | | | | | |
| % F oral | | | | | 1.36% | 1.16% | | | | | | |

^{a)}Plasma t_{1/2} values calculated: Group 4 (IV), 0.083 to 1.0 hr. Group 6 (Inh), 0.333 to 3.333 hr
NR = Not reportable due to an inability to construction a plasma concentration-time profile or characterize the elimination phase

### II. Inhalation Delivery of DMT by Nebulization

DMT hydrochloride will be formulated for nebulization by dissolving in water (buffered to pH 7 by isotonic phosphate buffer) to a concentration of 5 mg/ml. The freshly prepared solution (5 ml) will be loaded into the Aerogen Solo (Aerogen Corp., Dangan, Ireland) mesh nebulizer with a palladium mesh and connected to a continuous nebulization tube set. The rate of delivery of the solution to the nebulizer compartment is set to 0.2-1 ml/min to enable a therapeutic dose delivery by titration. The nebulized aerosol will be delivered to a patient via a face mask for a period of 5-20 min. For the first administration, the procedure will be terminated as soon as patients achieve the onset of psychedelic effects as determined by the patient's reporting and EEG live readouts.

### III. Inhalation Delivery of DMT by Metered-Dose Inhaler

DMT free base will be dissolved in HFA propellant 227 (20 mg/ml) and loaded in the pressurized stainless steel cannister (V=14 ml) of the metered dose inhaler equipped with a Bespak's BK357 valve and actuator (orifice d=0.22 mm) by Recipharm chosen to deliver 0.1 ml as a standard drug dose. In order to achieve dose titration, patients will be administered up to 10 drug doses every 2 min over a period of 20 min. For the first administration, the procedure will be terminated as soon as patients achieve the onset of psychedelic effects as determined by the patient's reporting and EEG live readouts.

Obviously, numerous modifications and variations of the disclosed methods and compositions are possible in light of the above teachings. It is therefore to be understood that within the scope of the appended claims, the compositions and methods may be practiced otherwise than as specifically described herein.

1. A method of delivering a psychedelic drug to a patient in need thereof, comprising:
administering an aerosol to the patient by inhalation,
wherein the aerosol comprises the psychedelic drug in a carrier, and the psychedelic drug is a compound of Formula (I) or a pharmaceutically acceptable salt, stereoisomer, solvate, or prodrug thereof 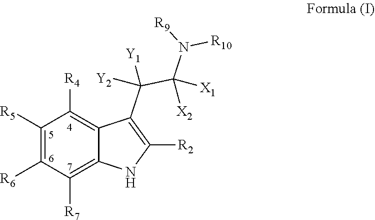
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl; and
wherein at least one of X₁, X₂, Y₁, Y₂, R₂, R₄, R₅, R₆, R₇, R₉, and R₁₀ comprises deuterium.

2-3. (canceled)

4. The method of claim 1, wherein X₁, X₂, Y₁, Y₂, R₉, and R₁₀ comprise deuterium.

5-6. (canceled)

7. The method of claim 1, wherein the compound of Formula (I) is at least one selected from the group consisting of 2-(1H-indol-3-yl)-N,N-bis(methyl-d₃)ethan-1-amine-1,1,2,2-d₄; 2-(1H-indol-3-yl)-N,N-bis(methyl-d₃)ethan-1-amine-1,1-d₂; 2-(5-methoxy-1H-indol-3-yl)-N,N-bis(methyl-d₃)ethan-1-amine-1,1,2,2-d₄; 2-(5-(methoxy-d₃)-1H-indol-3-yl)-N,N-dimethylethan-1-amine-1,1-d₂; and 2-(5-(methoxy-d₃)-1H-indol-3-yl)-N,N-bis(methyl-d₃)ethan-1-amine-1,1,2,2-d₄; or a pharmaceutically acceptable salt, solvate, or prodrug thereof.

8. The method of claim 1, wherein the psychedelic drug is a fumarate salt, benzoate salt, salicylate salt, or succinate salt of at least one selected from the group consisting of 2-(1H-indol-3-yl)-N,N-bis(methyl-d₃)ethan-1-amine-1,1,2,2-d₄; 2-(1H-indol-3-yl)-N,N-bis(methyl-d₃)ethan-1-amine-1,1-d₂; 2-(5-methoxy-1H-indol-3-yl)-N,N-bis(methyl-d₃)ethan-1-amine-1,1,2,2-d₄; 2-(5-(methoxy-d₃)-1H-indol-3-yl)-N,N-dimethylethan-1-amine-1,1-d₂; and 2-(5-(methoxy-d₃)-1H-indol-3-yl)-N,N-bis(methyl-d₃)ethan-1-amine-1,1,2,2-d₄.

9. The method of claim 1, wherein the psychedelic drug is a psychedelic drug mixture of at least two compounds of Formula (I), the psychedelic drug mixture comprising (i) 2-(1H-indol-3-yl)-N,N-bis(methyl-d₃)ethan-1-amine-1,1,2,2-d₄, or a pharmaceutically acceptable salt, solvate, or prodrug thereof, (ii) one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof.

10. The method of claim 9, wherein the psychedelic drug mixture comprises (i) from 60% to 99% by weight of 2-(1H-indol-3-yl)-N,N-bis(methyl-d₃)ethan-1-amine-1,1,2,2-d₄, or a pharmaceutically acceptable salt, solvate, or prodrug thereof, based on a total weight of the psychedelic drug mixture; (ii) from 1% to 40% by weight, in sum, of one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof, based on a total weight of the psychedelic drug mixture; and (iii) from 0% by weight to less than 10% by weight, in sum, of one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-i-amine-1,2-d₂, or a pharmaceutically acceptable salt, stereoisomer, solvate, or prodrug thereof, based on a total weight of the psychedelic drug mixture.

11. The method of claim 1, wherein the carrier is air, oxygen, or a mixture of helium and oxygen.

12-17. (canceled)

18. The method of claim 1, wherein the aerosol is a mist.

19. The method of claim 1, wherein the aerosol is prepared by nebulization of the psychedelic drug.

20. The method of claim 19, wherein the nebulization is performed with a device selected from the group consisting of a jet nebulizer, an ultrasonic nebulizer, a breath-actuated nebulizer, and a vibrating mesh nebulizer.

21-23. (canceled)

24. A method of treating a central nervous system (CNS) disorder or psychological disorder in a patient in need thereof, comprising:
administering to the patient, via inhalation, an aerosol comprising a psychedelic drug in a carrier,
wherein the psychedelic drug is a compound of Formula (I) or a pharmaceutically acceptable salt, stereoisomer, solvate, or prodrug thereof 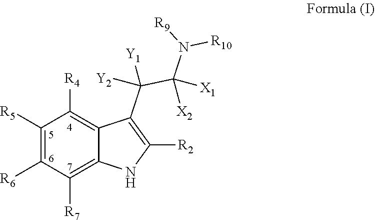
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl, and
wherein at least one of X₁, X₂, Y₁, Y₂, R₂, R₄, R₅, R₆, R₇, R₉, and R₁₀ comprises deuterium.

25-26. (canceled)

27. The method of claim 24, wherein X₁, X₂, Y₁, Y₂, R₉, and R₁₀ comprise deuterium.

28-29. (canceled)

30. The method of claim 24, wherein the compound of Formula (I) is at least one selected from the group consisting of 2-(1H-indol-3-yl)-N,N-bis(methyl-d₃)ethan-1-amine-1,1,2,2-d₄; 2-(1H-indol-3-yl)-N,N-bis(methyl-d₃)ethan-1-amine-1,1-d₂; 2-(5-methoxy-1H-indol-3-yl)-N,N-bis(methyl-d₃)ethan-1-amine-1,1,2,2-d₄; 2-(5-(methoxy-d₃)-1H-indol-3-yl)-N,N-dimethylethan-1-amine-1,1-d₂; and 2-(5-(methoxy-d₃)-1H-indol-3-yl)-N,N-bis(methyl-d₃)ethan-1-amine-1,1,2,2-d₄; or a pharmaceutically acceptable salt, solvate, or prodrug thereof.

31-40. (canceled)

41. The method of claim 24, wherein the aerosol is a mist.

42. The method of claim 24, wherein the aerosol is prepared by nebulization of the psychedelic drug.

43. The method of claim 42, wherein the nebulization is performed with a device selected from the group consisting of a jet nebulizer, an ultrasonic nebulizer, a breath-actuated nebulizer, and a vibrating mesh nebulizer.

44-49. (canceled)

50. The method of claim 24, wherein the CNS disorder or a psychological disorder is generalized anxiety disorder (GAD).

51. The method of claim 24, wherein the CNS disorder or a psychological disorder is generalized anxiety disorder (GAD) with depression.

52-53. (canceled)

54. A method of delivering a psychedelic drug to a patient in need thereof, comprising:
administering a dry powder to the patient by inhalation via a dry powder inhaler,
wherein the dry powder comprises the psychedelic drug, and the psychedelic drug is a compound of Formula (I) or a pharmaceutically acceptable salt, stereoisomer, solvate, or prodrug thereof 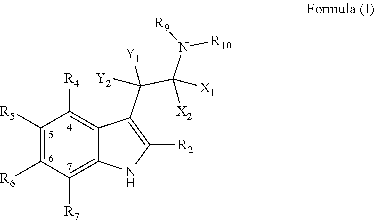
wherein:
X₁ and X₂ are independently selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
Y₁ and Y₂ are independently selected from the group consisting of hydrogen and deuterium;
R₂ is selected from the group consisting of hydrogen, deuterium, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl;
R₄ and R₅ are independently selected from the group consisting of hydrogen, deuterium, hydroxyl, unsubstituted or substituted alkyl, and unsubstituted or substituted alkoxy;
R₆ and R₇ are independently selected from the group consisting of hydrogen, deuterium, and halogen; and
R₉ and R₁₀ are independently selected from the group consisting of hydrogen, unsubstituted or substituted alkyl, unsubstituted or substituted alkenyl, unsubstituted or substituted alkynyl, unsubstituted or substituted cycloalkyl, unsubstituted or substituted heterocycloalkyl, unsubstituted or substituted aryl, and unsubstituted or substituted heteroaryl.

55. The method of claim 54, wherein the psychedelic drug is a psychedelic drug mixture of at least two compounds of Formula (I), the psychedelic drug mixture comprising (i) 2-(1H-indol-3-yl)-N,N-bis(methyl-d₃)ethan-1-amine-1,1,2,2-d₄, or a pharmaceutically acceptable salt, solvate, or prodrug thereof, (ii) one or more of 2-(1H-indol-3-yl)-N,N-bis(methyl-d₃)ethan-1-amine-1,2,2-d₃ and 2-(1H-indol-3-yl)-N,N-bis(methyl-d₃)ethan-1-amine-1,1,2-d₃, or a pharmaceutically acceptable salt, stereoisomer, solvate, or prodrug thereof; and optionally (iii) one or more of 2-(1H-indol-3-yl)-N,N-bis(methyl-d₃)ethan-1-amine-1,1-d₂, 2-(1H-indol-3-yl)-N,N-bis(methyl-d₃)ethan-1-amine-2,2-d₂, and 2-(1H-indol-3-yl)-N,N-bis(methyl-d₃)ethan-1-amine-1,2-d₂, or a pharmaceutically acceptable salt, stereoisomer, solvate, or prodrug thereof.

56-58. (canceled)
